SEQUENCE LISTING (133 sequences)

SEQ ID NO: 1 - protein, 130 aa, Artificial Sequence
  VEGFA-binding single domain antibody 13A6
SEVQLVESGGGLVQPGGSLRLSCAISGFSLAATDIDWVRQAPGKGLEWVARIFPSAGSTDYADSVKGRFTISADTSKNTVYLQMNSLRAEDTAVYYCGRSDSYSYNVFLHNPTYTALDYRGQGTLVTVSS

SEQ ID NO: 2 - protein, 6 aa, Artificial Sequence
  VEGFA-binding single domain antibody 13A6, 16A6.1 CDR1
SLAATD

SEQ ID NO: 3 - protein, 8 aa, Artificial Sequence
  VEGFA-binding single domain antibody 13A6 CDR2
FPSAGSTD

SEQ ID NO: 4 - protein, 21 aa, Artificial Sequence
  VEGFA-binding single domain antibody 13A6 CDR3
GRSDSYSYNVFLHNPTYTALD

SEQ ID NO: 5 - protein, 130 aa, Artificial Sequence
  VEGFA-binding single domain antibody 16A2.1
SEVQLVESGGGLVQPGGSLRLSCAISGFALAETDIDWVRQAPGKGLEWVARIFSSGGNTDYADSVKGRFTISADTSKNTVYLQMNSLRAEDTAVYYCGRSDSIAYNVILPNSTYTARDYRGQGTLVTVSS

SEQ ID NO: 6 - protein, 6 aa, Artificial Sequence
  VEGFA-binding single domain antibody 16A2.1 CDR1
ALAETD

SEQ ID NO: 7 - protein, 8 aa, Artificial Sequence
  VEGFA-binding single domain antibody 16A2.1 CDR2
FSSGGNTD

SEQ ID NO: 8 - protein, 21 aa, Artificial Sequence
  VEGFA-binding single domain antibody 16A2.1 CDR3
GRSDSIAYNVILPNSTYTARD

SEQ ID NO: 9 - protein, 130 aa, Artificial Sequence
  VEGFA-binding single domain antibody 16A2.1 CDR3
SEVQLVESGGGLVQPGGSLRLSCAISGFSLAATDIDWVRQAPGKGLEWVARIFPFSGSTDYTDSVKGRFTISADTSKNTVYLQMNSLRAEDTAVYYCGRSDSYSYNVFLHDPAYTALDYRGQGTLVTVSS

SEQ ID NO: 10 - protein, 8 aa, Artificial Sequence
  VEGFA-binding single domain antibody 16A6.1 CDR2
FPFSGSTD

SEQ ID NO: 11 - protein, 21 aa, Artificial Sequence
  VEGFA-binding single domain antibody 16A6.1 CDR3
GRSDSYSYNVFLHDPAYTALD

SEQ ID NO: 12 - protein, 130 aa, Artificial Sequence
  VEGFA-binding single domain antibody 20A2.1
SEVQLVESGGGLVQPGGSLRLSCAISGFPLADTDIDWVRQAPGKGLEWVARIFSSYGSTDYADSVKGRFTISADTSKNTVYLQMNSLRAEDTAVYYCGRSDSIAYNIILPASTYSSRDYRGQGTLVTVSS

SEQ ID NO: 13 - protein, 6 aa, Artificial Sequence
  VEGFA-binding single domain antibody 20A2.1 CDR1
PLADTD

SEQ ID NO: 14 - protein, 8 aa, Artificial Sequence
  VEGFA-binding single domain antibody 20A2.1 CDR2
FSSYGSTD

SEQ ID NO: 15 - protein, 21 aa, Artificial Sequence
  VEGFA-binding single domain antibody 20A2.1 CDR3
GRSDSIAYNIILPASTYSSRD

SEQ ID NO: 16 - protein, 130 aa, Artificial Sequence
  VEGFA-binding single domain antibody 20A3.1
SEVQLVESGGGLVQPGGSLRLSCAISGFSLADTDIDWVRQAPGKGLEWVARIFSSSGSTYYADSVKGRFTISADTSKNTVYLQMNSLRAEDTAVYYCDRADSVGYNVIFPGSTYSRSGYRGQGTLVTVSS

SEQ ID NO: 17 - protein, 6 aa, Artificial Sequence
  VEGFA-binding single domain antibody 20A3.1 CDR1
SLADTD

SEQ ID NO: 18 - protein, 8 aa, Artificial Sequence
  VEGFA-binding single domain antibody 20A3.1 CDR2
FSSSGSTY

SEQ ID NO: 19 - protein, 21 aa, Artificial Sequence
  VEGFA-binding single domain antibody 20A3.1 CDR3
DRADSVGYNVIFPGSTYSRSG

SEQ ID NO: 20 - protein, 21 aa, Artificial Sequence
  VEGFA-binding single domain antibody 21A1.1
DRADSVGYNVIFPGSTYSRSG

SEQ ID NO: 21 - protein, 6 aa, Artificial Sequence
  VEGFA-binding single domain antibody 21A1.1 CDR1
ALATTD

SEQ ID NO: 22 - protein, 8 aa, Artificial Sequence
  VEGFA-binding single domain antibody 21A1.1 CDR2
FNPSDFTD

SEQ ID NO: 23 - protein, 21 aa, Artificial Sequence
  VEGFA-binding single domain antibody 21A1.1 CDR3
GRPAVYGYNIFLDSPTYDASQ

SEQ ID NO: 24 - protein, 130 aa, Artificial Sequence
  VEGFA-binding single domain antibody 21A8.1
SEVQLVESGGGLVQPGGSLRLSCAISGFSVDATDIDWVRQAPGKGLEWVARIFSPSGFTDYADSVKGRFTISADTSKNTVYLQMNSLRAEDTAVYYCDRPEFYAYNIFHDPRTVPAGNYRGQGTLVTVSS

SEQ ID NO: 25 - protein, 6 aa, Artificial Sequence
  VEGFA-binding single domain antibody 21A8.1 CDR1
SVDATD

SEQ ID NO: 26 - protein, 8 aa, Artificial Sequence
  VEGFA-binding single domain antibody 21A8.1 CDR2
FSPSGFTD

SEQ ID NO: 27 - protein, 21 aa, Artificial Sequence
  VEGFA-binding single domain antibody 21A8.1 CDR3
DRPEFYAYNIFHDPRTVPAGN

SEQ ID NO: 28 - protein, 130 aa, Artificial Sequence
  VEGFA-binding single domain antibody 21D9.1
SEVQLVESGGGLVQPGGSLRLSCAISGFSLGATDIDWVRQAPGKGLEWVARIFSPSDFTDYADSVKGRFTISADTSKNTVYLQMNSLRAEDTAVYYCSRPDTYAYNIFPHTASHGAFNYRGQGTLVTVSS

SEQ ID NO: 29 - protein, 6 aa, Artificial Sequence
  VEGFA-binding single domain antibody 21D9.1 CDR1
SLGATD

SEQ ID NO: 30 - protein, 8 aa, Artificial Sequence
  VEGFA-binding single domain antibody 21D9.1 CDR2
FSPSDFTD

SEQ ID NO: 31 - protein, 21 aa, Artificial Sequence
  VEGFA-binding single domain antibody 21D9.1 CDR3
SRPDTYAYNIFPHTASHGAFN

SEQ ID NO: 32 - protein, 130 aa, Artificial Sequence
  VEGFA-binding single domain antibody 21E6.1
SEVQLVESGGGLVQPGGSLRLSCAISGFTLAATDIDWVRQAPGKGLEWVARIFTPTGFSDYADSVKGRFTISADTSKNTVYLQMNSLRAEDTAVYYCGIPELYAYNIFMDIPAYNDVYYRGQGTLVTVSS

SEQ ID NO: 33 - protein, 6 aa, Artificial Sequence
  VEGFA-binding single domain antibody 21E6.1 CDR1
TLAATD

SEQ ID NO: 34 - protein, 8 aa, Artificial Sequence
  VEGFA-binding single domain antibody 21E6.1 CDR2
FTPTGFSD

SEQ ID NO: 35 - protein, 21 aa, Artificial Sequence
  VEGFA-binding single domain antibody 21E6.1 CDR3
GIPELYAYNIFMDIPAYNDVY

SEQ ID NO: 36 - protein, 130 aa, Artificial Sequence
  VEGFA-binding single domain antibody 23D5.1
SEVQLVESGGGLVQPGGSLRLSCAISGFDLATTDIDWVRQAPGKGLEWVARIFRPSDFTDYADSVKGRFTISADTSKNTVYLQMNSLRAEDTAVYYCVRPDSYAYNILFVTRTFNALHYRGQGTLVTVSS

SEQ ID NO: 37 - protein, 6 aa, Artificial Sequence
  VEGFA-binding single domain antibody 23D5.1 CDR1
DLATTD

SEQ ID NO: 38 - protein, 8 aa, Artificial Sequence
  VEGFA-binding single domain antibody 23D5.1 CDR2
FRPSDFTD

SEQ ID NO: 39 - protein, 21 aa, Artificial Sequence
  VEGFA-binding single domain antibody 23D5.1 CDR3
VRPDSYAYNILFVTRTFNALH

SEQ ID NO: 40 - protein, 28 aa, Artificial Sequence
  FW_FR1
SEVQLVESGGGLVQPGGSLRLSCAISGF

SEQ ID NO: 41 - protein, 18 aa, Artificial Sequence
  FW_FR2
IDWVRQAPGKGLEWVARI

SEQ ID NO: 42 - protein, 37 aa, Artificial Sequence
  FW_FR3_13A6, 16A2.1, 20A2.1, 20A3.1, 21A1.1, 21A8.1, 21D9.1, 21E6.1
YADSVKGRFTISADTSKNTVYLQMNSLRAEDTAVYYC

SEQ ID NO: 43 - protein, 37 aa, Artificial Sequence
  FW_FR3_16A6.1
YTDSVKGRFTISADTSKNTVYLQMNSLRAEDTAVYYC

SEQ ID NO: 44 - protein, 12 aa, Artificial Sequence
  FW_FR4
YRGQGTLVTVSS

SEQ ID NO: 45 - protein, 6 aa, Artificial Sequence
  CDR1 ConAll
  MISC_FEATURE (1)..(1): Wherein Xaa = S, A, P, T or D
  MISC_FEATURE (2)..(2): Wherein Xaa = L or V
  MISC_FEATURE (3)..(3): Wherein Xaa = A, D or G
  MISC_FEATURE (4)..(4): Wherein Xaa = A, D, T or E
XXXXTD

SEQ ID NO: 46 - protein, 8 aa, Artificial Sequence
  CDR2 ConAll
  MISC_FEATURE (2)..(2): Wherein Xaa = S, P, N, T
  MISC_FEATURE (3)..(3): Wherein Xaa = P, S or F
  MISC_FEATURE (4)..(4): Wherein Xaa =S, Y, G, A or T
  MISC_FEATURE (5)..(5): Wherein Xaa = G or D
  MISC_FEATURE (6)..(6): Wherein Xaa = F, S or N
  MISC_FEATURE (7)..(7): Wherein Xaa = T or S
  MISC_FEATURE (8)..(8): Wherein Xaa = D or Y
FXXXXXXX

SEQ ID NO: 47 - protein, 37 aa, Artificial Sequence
  FW_FR3 ConAll
  MISC_FEATURE (2)..(2): Wherein Xaa = A or T
YXDSVKGRFTISADTSKNTVYLQMNSLRAEDTAVYYC

SEQ ID NO: 48 - protein, 21 aa, Artificial Sequence
  CDR3 ConAll
  MISC_FEATURE (1)..(1): Wherein Xaa = G, D, S or V
  MISC_FEATURE (2)..(2): Wherein Xaa = R or I
  MISC_FEATURE (3)..(3): Wherein Xaa = P, S or A
  MISC_FEATURE (4)..(4): Wherein Xaa = D, A or E
  MISC_FEATURE (5)..(5): Wherein Xaa = S, V, L, F or T
  MISC_FEATURE (6)..(6): Wherein Xaa = Y, V or I
  MISC_FEATURE (7)..(7): Wherein Xaa = A, G or S
  MISC_FEATURE (10)..(10): Wherein Xaa = I or V
  MISC_FEATURE (11)..(11): Wherein Xaa = F, I or L
  MISC_FEATURE (12)..(12): Wherein Xaa = L, F M, H or P
  MISC_FEATURE (13)..(13): Wherein Xaa = D, P, H or V
  MISC_FEATURE (14)..(14): Wherein Xaa = N, S, G, A, D, I, P or T
  MISC_FEATURE (15)..(15): Wherein Xaa = P, S, R or A
  MISC_FEATURE (16)..(16): Wherein Xaa = T, A or S
  MISC_FEATURE (17)..(17): Wherein Xaa = Y, V, H or F
  MISC_FEATURE (18)..(18): Wherein Xaa = T, D, S, N, P or G
  MISC_FEATURE (19)..(19): Wherein Xaa =  A, S, R or D
  MISC_FEATURE (20)..(20): Wherein Xaa =  S, L, R, V, G or F
  MISC_FEATURE (21)..(21): Wherein Xaa =  D, Q, N, G, Y or H
XXXXXXXYNXXXXXXXXXXXX

SEQ ID NO: 49 - protein, 130 aa, Artificial Sequence
  Con SeqAll
  MISC_FEATURE (29)..(29): Wherein Xaa = S, A, P, T or D
  MISC_FEATURE (30)..(30): Wherein Xaa = L or V
  MISC_FEATURE (31)..(31): Wherein Xaa = A, D or G
  MISC_FEATURE (32)..(32): Wherein Xaa = A, D, T or E
  MISC_FEATURE (54)..(54): Wherein Xaa = S, P, N, T or R
  MISC_FEATURE (55)..(55): Wherein Xaa = P, S or F
  MISC_FEATURE (56)..(56): Wherein Xaa = S, Y, G, A or T
  MISC_FEATURE (57)..(57): Wherein Xaa = G or D
  MISC_FEATURE (58)..(58): Wherein Xaa =  F, S or N
  MISC_FEATURE (59)..(59): Wherein Xaa = T or S
  MISC_FEATURE (60)..(60): Wherein Xaa = D or Y
  MISC_FEATURE (62)..(62): Wherein Xaa = A or T
  MISC_FEATURE (98)..(98): Wherein Xaa = G, D, S or V
  MISC_FEATURE (99)..(99): Wherein Xaa = R or I
  MISC_FEATURE (100)..(100): Wherein Xaa = P, S or A
  MISC_FEATURE (101)..(101): Wherein Xaa = D, A or E
  MISC_FEATURE (102)..(102): Wherein Xaa = S, V, L, F or T
  MISC_FEATURE (103)..(103): Wherein Xaa = Y, V or I
  MISC_FEATURE (104)..(104): Wherein Xaa = A, G or S
  MISC_FEATURE (107)..(107): Wherein Xaa =  I or V
  MISC_FEATURE (108)..(108): Wherein Xaa = F, I or L
  MISC_FEATURE (109)..(109): Wherein Xaa = L, F M, H or P
  MISC_FEATURE (110)..(110): Wherein Xaa = D, P, H or V
  MISC_FEATURE (111)..(111): Wherein Xaa = N, S, G, A, D, I, P or T
  MISC_FEATURE (112)..(112): Wherein Xaa = P, S, R or A
  MISC_FEATURE (113)..(113): Wherein Xaa = T, A or S
  MISC_FEATURE (114)..(114): Wherein Xaa = Y, V, H or F
  MISC_FEATURE (115)..(115): Wherein Xaa = T, D, S, N, P or G
  MISC_FEATURE (116)..(116): Wherein Xaa = A, S, R or D
  MISC_FEATURE (117)..(117): Wherein Xaa = S, L, R, V, G or F
  MISC_FEATURE (118)..(118): Wherein Xaa = D, Q, N, G, Y or H
SEVQLVESGGGLVQPGGSLRLSCAISGFXXXXTDIDWVRQAPGKGLEWVARIFXXXXXXXYXDSVKGRFTISADTSKNTVYLQMNSLRAEDTAVYYCXXXXXXXYNXXXXXXXXXXXXYRGQGTLVTVSS

SEQ ID NO: 50 - protein, 6 aa, Artificial Sequence
  CDR1 Con16A2.1derived
  MISC_FEATURE (1)..(1): Wherein Xaa = P, S or A
  MISC_FEATURE (4)..(4): Wherein Xaa = D or E
XLAXTD

SEQ ID NO: 51 - protein, 8 aa, Artificial Sequence
  CDR2 Con16A2.1derived
  MISC_FEATURE (4)..(4): Wherein Xaa = Y, S or G
  MISC_FEATURE (6)..(6): Wherein Xaa = S or N
  MISC_FEATURE (8)..(8): Wherein Xaa = D or Y
FSSXGXTX

SEQ ID NO: 52 - protein, 21 aa, Artificial Sequence
  CDR3 Con16A2.1derived
  MISC_FEATURE (1)..(1): Wherein Xaa = G or D
  MISC_FEATURE (3)..(3): Wherein Xaa = S or A
  MISC_FEATURE (6)..(6): Wherein Xaa = I or V
  MISC_FEATURE (7)..(7): Wherein Xaa = A or G
  MISC_FEATURE (10)..(10): Wherein Xaa = V or I
  MISC_FEATURE (12)..(12): Wherein Xaa = L or F
  MISC_FEATURE (14)..(14): Wherein Xaa = G, A or N
  MISC_FEATURE (18)..(18): Wherein Xaa = S or T
  MISC_FEATURE (19)..(19): Wherein Xaa = R, S or A
  MISC_FEATURE (20)..(20): Wherein Xaa = R or S
  MISC_FEATURE (21)..(21): Wherein Xaa = D or G
XRXDSXXYNXIXPXSTYXXXX

SEQ ID NO: 53 - protein, 130 aa, Artificial Sequence
  Con Seq16A2.1derived
  MISC_FEATURE (29)..(29): Wherein Xaa = P, S or A
  MISC_FEATURE (32)..(32): Wherein Xaa = D or E
  MISC_FEATURE (56)..(56): Wherein Xaa = Y, S or G
  MISC_FEATURE (58)..(58): Wherein Xaa = S or N
  MISC_FEATURE (60)..(60): Wherein Xaa =  D or Y
  MISC_FEATURE (98)..(98): Wherein Xaa =  G or D
  MISC_FEATURE (100)..(100): Wherein Xaa =  S or A
  MISC_FEATURE (103)..(103): Wherein Xaa =  I or V
  MISC_FEATURE (104)..(104): Wherein Xaa =  A or G
  MISC_FEATURE (107)..(107): Wherein Xaa =  V or I
  MISC_FEATURE (109)..(109): Wherein Xaa =  L or F
  MISC_FEATURE (111)..(111): Wherein Xaa = G, A or N
  MISC_FEATURE (115)..(115): Wherein Xaa = S or T
  MISC_FEATURE (116)..(116): Wherein Xaa = R, S or A
  MISC_FEATURE (117)..(117): Wherein Xaa = R or S
  MISC_FEATURE (118)..(118): Wherein Xaa = D or G
SEVQLVESGGGLVQPGGSLRLSCAISGFXLAXTDIDWVRQAPGKGLEWVARIFSSXGXTXYADSVKGRFTISADTSKNTVYLQMNSLRAEDTAVYYCXRXDSXXYNXIXPXSTYXXXXYRGQGTLVTVSS

SEQ ID NO: 54 - protein, 6 aa, Artificial Sequence
  CDR1 Con21/23
  MISC_FEATURE (1)..(1): Wherein Xaa = A, S, T or D
  MISC_FEATURE (2)..(2): Wherein Xaa = L or V
  MISC_FEATURE (3)..(3): Wherein Xaa = A, D or G
  MISC_FEATURE (4)..(4): Wherein Xaa = A or T
XXXXTD

SEQ ID NO: 55 - protein, 8 aa, Artificial Sequence
  CDR2 Con21/23
  MISC_FEATURE (2)..(2): Wherein Xaa = S, N or T
  MISC_FEATURE (4)..(4): Wherein Xaa = S or T
  MISC_FEATURE (5)..(5): Wherein Xaa = D or G
  MISC_FEATURE (7)..(7): Wherein Xaa = T or S
FXPXXFXD

SEQ ID NO: 56 - protein, 21 aa, Artificial Sequence
  CDR3 Con21/23
  MISC_FEATURE (1)..(1): Wherein Xaa = G, D, S or V
  MISC_FEATURE (2)..(2): Wherein Xaa = R or I
  MISC_FEATURE (4)..(4): Wherein Xaa = A, E or D
  MISC_FEATURE (5)..(5): Wherein Xaa = V, T, L, S or F
  MISC_FEATURE (7)..(7): Wherein Xaa = A or G
  MISC_FEATURE (11)..(11): Wherein Xaa = F or L
  MISC_FEATURE (12)..(12): Wherein Xaa = L, H, P or M
  MISC_FEATURE (13)..(13): Wherein Xaa = D, H or V
  MISC_FEATURE (14)..(14): Wherein Xaa = S, R, P or I
  MISC_FEATURE (15)..(15): Wherein Xaa = P, R or A
  MISC_FEATURE (16)..(16): Wherein Xaa = T, S or A
  MISC_FEATURE (17)..(17): Wherein Xaa = Y, V, H or F
  MISC_FEATURE (18)..(18): Wherein Xaa = D, N, G or P
  MISC_FEATURE (19)..(19): Wherein Xaa = A or D
  MISC_FEATURE (20)..(20): Wherein Xaa = S, G, F, V or L
  MISC_FEATURE (21)..(21): Wherein Xaa = Q, N, Y or H
XXPXXYXYNIXXXXXXXXXXX

SEQ ID NO: 57 - protein, 130 aa, Artificial Sequence
  Con Seq21/23
  MISC_FEATURE (29)..(29): Wherein Xaa = A, S, T or D
  MISC_FEATURE (30)..(30): Wherein Xaa = L or V
  MISC_FEATURE (31)..(31): Wherein Xaa = A, D or G
  MISC_FEATURE (32)..(32): Wherein Xaa = A or T
  MISC_FEATURE (54)..(54): Wherein Xaa = S, N or T
  MISC_FEATURE (56)..(56): Wherein Xaa = S or T
  MISC_FEATURE (57)..(57): Wherein Xaa = D or G
  MISC_FEATURE (59)..(59): Wherein Xaa = T or S
  MISC_FEATURE (98)..(98): Wherein Xaa = G, D, S or V
  MISC_FEATURE (99)..(99): Wherein Xaa = R or I
  MISC_FEATURE (101)..(101): Wherein Xaa = A, E or D
  MISC_FEATURE (102)..(102): Wherein Xaa = V, T, L, S or F
  MISC_FEATURE (104)..(104): Wherein Xaa =  A or G
  MISC_FEATURE (108)..(108): Wherein Xaa = F or L
  MISC_FEATURE (109)..(109): Wherein Xaa = L, H, P or M
  MISC_FEATURE (110)..(110): Wherein Xaa = D, H or V
  MISC_FEATURE (111)..(111): Wherein Xaa = S, R, P or I
  MISC_FEATURE (112)..(112): Wherein Xaa = P, R or A
  MISC_FEATURE (113)..(113): Wherein Xaa = T, S or A
  MISC_FEATURE (114)..(114): Wherein Xaa = Y, V, H or F
  MISC_FEATURE (115)..(115): Wherein Xaa = D, N, G or P
  MISC_FEATURE (116)..(116): Wherein Xaa = A or D
  MISC_FEATURE (117)..(117): Wherein Xaa = S, G, F, V or L
  MISC_FEATURE (118)..(118): Wherein Xaa = Q, N, Y or H
SEVQLVESGGGLVQPGGSLRLSCAISGFXXXXTDIDWVRQAPGKGLEWVARIFXPXXFXDYADSVKGRFTISADTSKNTVYLQMNSLRAEDTAVYYCXXPXXYXYNIXXXXXXXXXXXYRGQGTLVTVSS

SEQ ID NO: 58 - protein, 34 aa, Artificial Sequence
  GS linker
GGGGSGGGGSGGGGSGGGGSGGGGSGGGGSGGGG

SEQ ID NO: 59 - protein, 294 aa, Artificial Sequence
  16A2.1x2
SEVQLVESGGGLVQPGGSLRLSCAISGFALAETDIDWVRQAPGKGLEWVARIFSSGGNTDYADSVKGRFTISADTSKNTVYLQMNSLRAEDTAVYYCGRSDSIAYNVILPNSTYTARDYRGQGTLVTVSSGGGGSGGGGSGGGGSGGGGSGGGGSGGGGSGGGGSEVQLVESGGGLVQPGGSLRLSCAISGFALAETDIDWVRQAPGKGLEWVARIFSSGGNTDYADSVKGRFTISADTSKNTVYLQMNSLRAEDTAVYYCGRSDSIAYNVILPNSTYTARDYRGQGTLVTVSS

SEQ ID NO: 60 - protein, 232 aa, Artificial Sequence
  VEGF206
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 61 - protein, 215 aa, Artificial Sequence
  VEGF189
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 62 - protein, 191 aa, Artificial Sequence
  VEGF165
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 63 - protein, 147 aa, Artificial Sequence
  VEGF121
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 64 - protein, 206 aa, Artificial Sequence
  Mature VEGF206
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 65 - protein, 189 aa, Artificial Sequence
  Mature VEGF189
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 66 - protein, 165 aa, Artificial Sequence
  Mature VEGF165
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 67 - protein, 121 aa, Artificial Sequence
  Mature VEGF121
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 68 - protein, 26 aa, Artificial Sequence
  VEGFA Signal Peptide
MNFLLSWVHWSLALLLYLHHAKWSQA

SEQ ID NO: 69 - protein, 330 aa, Artificial Sequence [sequence omitted]

SEQ ID NO: 70 - protein, 98 aa, Artificial Sequence
  CH1 IgG1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 71 - protein, 12 aa, Artificial Sequence
  Hinge IgG1
EPKSCDKTHTCP

SEQ ID NO: 72 - protein, 113 aa, Artificial Sequence
  CH2 IgG1
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 73 - protein, 107 aa, Artificial Sequence
  CH3 IgG1
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 231 aa, Artificial Sequence
  Ranibizumab Heavy Chain
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 75 - protein, 214 aa, Artificial Sequence
  Ranibizumab Light Chain
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 453 aa, Artificial Sequence
  Bevacizumab Heavy Chain
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 214 aa, Artificial Sequence
  Bevacizumab Light Chain
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 117 aa, Artificial Sequence
  Na?ve synthetic DotBody phage display library 1
SEVQLVESGGGLVQPGGSLRLSSAISGFSISSTSIDWVRQAPGKGLEWVARISPSSGSTSYADSVKGRFTISADTSKNTVYLQMNSLRAEDTAVYYTGRSSSAMDYRGQGTLVTVSS

SEQ ID NO: 79 - protein, 117 aa, Artificial Sequence
  Na?ve synthetic DotBody phage display library 2
SEVQLVESGGGLVQPGGSLRLSCAISGFSISSTSIDWVRQAPGKGLEWVARISPSSGSTSYADSVKGRFTISADTSKNTVYLQMNSLRAEDTAVYYCGRSSSAMDYRGQGTLVTVSS

SEQ ID NO: 80 - DNA, 57 bp, Artificial Sequence
  Primer H1
  misc_feature (21)..(21): n = 0.2G + 0.2A + 0.5T + 0.1C
  misc_feature (22)..(22): n = 0.4A + 0.2T + 0.4C
  misc_feature (23)..(23): n = 0.1G + 0.9C
  misc_feature (24)..(24): n is a, c, g, or t
  misc_feature (27)..(27): n = 0.4A + 0.2T + 0.4C
  misc_feature (28)..(28): n = 0.1G + 0.9C
  misc_feature (29)..(29): n = 0.2G + 0.2A + 0.5T + 0.1C
  misc_feature (30)..(30): n = 0.4A + 0.2T + 0.4C
  misc_feature (31)..(31): n = 0.1G + 0.9C
  misc_feature (32)..(32): n is a, c, g, or t
  misc_feature (35)..(35): n = 0.2G + 0.2A + 0.5T + 0.1C
  misc_feature (36)..(36): n = 0.4A + 0.2T + 0.4C
  misc_feature (37)..(37): n = 0.1G + 0.9C
  misc_feature (38)..(38): n is a, c, g, or t
cctctgcaatttctggcttcnnnnttnnnnnnactnnnatagactgggtgcgtcagg

SEQ ID NO: 81 - DNA, 65 bp, Artificial Sequence
  Primer H2
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
ctggaatgggttgcaaggattnnncctnnnnnnggtnnnactnnntatgccgatagcgtcaaggg

SEQ ID NO: 82 - DNA, 61 bp, Artificial Sequence
  Primer H3.6
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (43)..(43): n is a, c, g, or t
gccgtctattatactgkccgcnnnnnnnnnnnnnnnnnngstntkgactaccggggtcaag

SEQ ID NO: 83 - DNA, 64 bp, Artificial Sequence
  Primer H3.7
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (46)..(46): n is a, c, g, or t
gccgtctattatactgkccgcnnnnnnnnnnnnnnnnnnnnngstntkgactaccggggtcaag

SEQ ID NO: 84 - DNA, 67 bp, Artificial Sequence
  Primer H3.8
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
  misc_feature (49)..(49): n is a, c, g, or t
gccgtctattatactgkccgcnnnnnnnnnnnnnnnnnnnnnnnngstntkgactaccggggtcaag

SEQ ID NO: 85 - DNA, 70 bp, Artificial Sequence
  Primer H3.9
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
  misc_feature (46)..(46): n = 0.2G + 0.2A + 0.5T + 0.1C
  misc_feature (47)..(47): n = 0.4A + 0.2T + 0.4C
  misc_feature (48)..(48): n = 0.1G + 0.9C
  misc_feature (52)..(52): n is a, c, g, or t
gccgtctattatactgkccgcnnnnnnnnnnnnnnnnnnnnnnnnnnngstntkgactaccggggtcaag

SEQ ID NO: 86 - DNA, 73 bp, Artificial Sequence
  Primer H3.10
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.4A + 0.2T + 0.4C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.4A + 0.2T + 0.4C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.4A + 0.2T + 0.4C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.4A + 0.2T + 0.4C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.4A + 0.2T + 0.4C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.4A + 0.2T + 0.4C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.4A + 0.2T + 0.4C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.4A + 0.2T + 0.4C
  misc_feature (46)..(46): n = 0.2G + 0.2A + 0.5T + 0.1C
  misc_feature (47)..(47): n = 0.4A + 0.2T + 0.4C
  misc_feature (48)..(48): n = 0.4A + 0.2T + 0.4C
  misc_feature (49)..(49): n = 0.2G + 0.2A + 0.5T + 0.1C
  misc_feature (50)..(50): n = 0.4A + 0.2T + 0.4C
  misc_feature (51)..(51): n = 0.4A + 0.2T + 0.4C
  misc_feature (55)..(55): n is a, c, g, or t
gccgtctattatactgkccgcnnnnnnnnnnnnnnnnnnnnnnnnnnnnnngstntkgactaccggggtcaag

SEQ ID NO: 87 - DNA, 76 bp, Artificial Sequence
  Primer H3.11
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
  misc_feature (46)..(46): n = 0.2G + 0.2A + 0.5T + 0.1C
  misc_feature (47)..(47): n = 0.4A + 0.2T + 0.4C
  misc_feature (48)..(48): n = 0.1G + 0.9C
  misc_feature (49)..(49): n = 0.2G + 0.2A + 0.5T + 0.1C
  misc_feature (50)..(50): n = 0.4A + 0.2T + 0.4C
  misc_feature (51)..(51): n = 0.1G + 0.9C
  misc_feature (52)..(52): n = 0.2G + 0.2A + 0.5T + 0.1C
  misc_feature (53)..(53): n = 0.4A + 0.2T + 0.4C
  misc_feature (54)..(54): n = 0.1G + 0.9C
  misc_feature (58)..(58): n is a, c, g, or t
gccgtctattatactgkccgcnnnnnnnnnnnnnnnnnnnnnnnnnnnnnnnnngstntkgactaccggggtcaag

SEQ ID NO: 88 - DNA, 79 bp, Artificial Sequence
  Primer H3.12
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
  misc_feature (46)..(46): n = 0.2G + 0.2A + 0.5T + 0.1C
  misc_feature (47)..(47): n = 0.4A + 0.2T + 0.4C
  misc_feature (48)..(48): n = 0.1G + 0.9C
  misc_feature (49)..(49): n = 0.2G + 0.2A + 0.5T + 0.1C
  misc_feature (50)..(50): n = 0.4A + 0.2T + 0.4C
  misc_feature (51)..(51): n = 0.1G + 0.9C
  misc_feature (52)..(52): n = 0.2G + 0.2A + 0.5T + 0.1C
  misc_feature (53)..(53): n = 0.4A + 0.2T + 0.4C
  misc_feature (54)..(54): n = 0.1G + 0.9C
  misc_feature (55)..(55): n = 0.2G + 0.2A + 0.5T + 0.1C
  misc_feature (56)..(56): n = 0.4A + 0.2T + 0.4C
  misc_feature (57)..(57): n = 0.1G + 0.9C
  misc_feature (61)..(61): n is a, c, g, or t
gccgtctattatactgkccgcnnnnnnnnnnnnnnnnnnnnnnnnnnnnnnnnnnnngstntkgactaccggggtcaag

SEQ ID NO: 89 - DNA, 82 bp, Artificial Sequence
  Primer H3.13
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
  misc_feature (46)..(46): n = 0.2G + 0.2A + 0.5T + 0.1C
  misc_feature (47)..(47): n = 0.4A + 0.2T + 0.4C
  misc_feature (48)..(48): n = 0.1G + 0.9C
  misc_feature (49)..(49): n = 0.2G + 0.2A + 0.5T + 0.1C
  misc_feature (50)..(50): n = 0.4A + 0.2T + 0.4C
  misc_feature (51)..(51): n = 0.1G + 0.9C
  misc_feature (52)..(52): n = 0.2G + 0.2A + 0.5T + 0.1C
  misc_feature (53)..(53): n = 0.4A + 0.2T + 0.4C
  misc_feature (54)..(54): n = 0.1G + 0.9C
  misc_feature (55)..(55): n = 0.2G + 0.2A + 0.5T + 0.1C
  misc_feature (56)..(56): n = 0.4A + 0.2T + 0.4C
  misc_feature (57)..(57): n = 0.1G + 0.9C
  misc_feature (58)..(58): n = 0.2G + 0.2A + 0.5T + 0.1C
  misc_feature (59)..(59): n = 0.4A + 0.2T + 0.4C
  misc_feature (60)..(60): n = 0.1G + 0.9C
  misc_feature (64)..(64): n is a, c, g, or t
gccgtctattatactgkccgcnnnnnnnnnnnnnnnnnnnnnnnnnnnnnnnnnnnnnnngstntkgactaccggggtcaag

SEQ ID NO: 90 - DNA, 85 bp, Artificial Sequence
  Primer H3.14
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
  misc_feature (46)..(46): n = 0.2G + 0.2A + 0.5T + 0.1C
  misc_feature (47)..(47): n = 0.4A + 0.2T + 0.4C
  misc_feature (48)..(48): n = 0.1G + 0.9C
  misc_feature (49)..(49): n = 0.2G + 0.2A + 0.5T + 0.1C
  misc_feature (50)..(50): n = 0.4A + 0.2T + 0.4C
  misc_feature (51)..(51): n = 0.1G + 0.9C
  misc_feature (52)..(52): n = 0.2G + 0.2A + 0.5T + 0.1C
  misc_feature (53)..(53): n = 0.4A + 0.2T + 0.4C
  misc_feature (54)..(54): n = 0.1G + 0.9C
  misc_feature (55)..(55): n = 0.2G + 0.2A + 0.5T + 0.1C
  misc_feature (56)..(56): n = 0.4A + 0.2T + 0.4C
  misc_feature (57)..(57): n = 0.1G + 0.9C
  misc_feature (58)..(58): n = 0.2G + 0.2A + 0.5T + 0.1C
  misc_feature (59)..(59): n = 0.4A + 0.2T + 0.4C
  misc_feature (60)..(60): n = 0.1G + 0.9C
  misc_feature (61)..(61): n = 0.2G + 0.2A + 0.5T + 0.1C
  misc_feature (62)..(62): n = 0.4A + 0.2T + 0.4C
  misc_feature (63)..(63): n = 0.1G + 0.9C
  misc_feature (67)..(67): n is a, c, g, or t
gccgtctattatactgkccgcnnnnnnnnnnnnnnnnnnnnnnnnnnnnnnnnnnnnnnnnnngstntkgactaccggggtcaag

SEQ ID NO: 91 - DNA, 88 bp, Artificial Sequence
  Primer H3.15
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
  misc_feature (46)..(46): n = 0.2G + 0.2A + 0.5T + 0.1C
  misc_feature (47)..(47): n = 0.4A + 0.2T + 0.4C
  misc_feature (48)..(48): n = 0.1G + 0.9C
  misc_feature (49)..(49): n = 0.2G + 0.2A + 0.5T + 0.1C
  misc_feature (50)..(50): n = 0.4A + 0.2T + 0.4C
  misc_feature (51)..(51): n = 0.1G + 0.9C
  misc_feature (52)..(52): n = 0.2G + 0.2A + 0.5T + 0.1C
  misc_feature (53)..(53): n = 0.4A + 0.2T + 0.4C
  misc_feature (54)..(54): n = 0.1G + 0.9C
  misc_feature (55)..(55): n = 0.2G + 0.2A + 0.5T + 0.1C
  misc_feature (56)..(56): n = 0.4A + 0.2T + 0.4C
  misc_feature (57)..(57): n = 0.1G + 0.9C
  misc_feature (58)..(58): n = 0.2G + 0.2A + 0.5T + 0.1C
  misc_feature (59)..(59): n = 0.4A + 0.2T + 0.4C
  misc_feature (60)..(60): n = 0.1G + 0.9C
  misc_feature (61)..(61): n = 0.2G + 0.2A + 0.5T + 0.1C
  misc_feature (62)..(62): n = 0.4A + 0.2T + 0.4C
  misc_feature (63)..(63): n = 0.1G + 0.9C
  misc_feature (64)..(64): n = 0.2G + 0.2A + 0.5T + 0.1C
  misc_feature (65)..(65): n = 0.4A + 0.2T + 0.4C
  misc_feature (66)..(66): n = 0.1G + 0.9C
  misc_feature (70)..(70): n is a, c, g, or t
gccgtctattatactgkccgcnnnnnnnnnnnnnnnnnnnnnnnnnnnnnnnnnnnnnnnnnnnnngstntkgactaccggggtcaag

SEQ ID NO: 92 - DNA, 91 bp, Artificial Sequence
  Primer H3.16
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
  misc_feature (46)..(46): n = 0.2G + 0.2A + 0.5T + 0.1C
  misc_feature (47)..(47): n = 0.4A + 0.2T + 0.4C
  misc_feature (48)..(48): n = 0.1G + 0.9C
  misc_feature (49)..(49): n = 0.2G + 0.2A + 0.5T + 0.1C
  misc_feature (50)..(50): n = 0.4A + 0.2T + 0.4C
  misc_feature (51)..(51): n = 0.1G + 0.9C
  misc_feature (52)..(52): n = 0.2G + 0.2A + 0.5T + 0.1C
  misc_feature (53)..(53): n = 0.4A + 0.2T + 0.4C
  misc_feature (54)..(54): n = 0.1G + 0.9C
  misc_feature (55)..(55): n = 0.2G + 0.2A + 0.5T + 0.1C
  misc_feature (56)..(56): n = 0.4A + 0.2T + 0.4C
  misc_feature (57)..(57): n = 0.1G + 0.9C
  misc_feature (58)..(58): n = 0.2G + 0.2A + 0.5T + 0.1C
  misc_feature (59)..(59): n = 0.4A + 0.2T + 0.4C
  misc_feature (60)..(60): n = 0.1G + 0.9C
  misc_feature (61)..(61): n = 0.2G + 0.2A + 0.5T + 0.1C
  misc_feature (62)..(62): n = 0.4A + 0.2T + 0.4C
  misc_feature (63)..(63): n = 0.1G + 0.9C
  misc_feature (64)..(64): n = 0.2G + 0.2A + 0.5T + 0.1C
  misc_feature (65)..(65): n = 0.4A + 0.2T + 0.4C
  misc_feature (66)..(66): n = 0.1G + 0.9C
  misc_feature (67)..(67): n = 0.2G + 0.2A + 0.5T + 0.1C
  misc_feature (68)..(68): n = 0.4A + 0.2T + 0.4C
  misc_feature (69)..(69): n = 0.1G + 0.9C
  misc_feature (73)..(73): n is a, c, g, or t
gccgtctattatactgkccgcnnnnnnnnnnnnnnnnnnnnnnnnnnnnnnnnnnnnnnnnnnnnnnnngstntkgactaccggggtcaag

SEQ ID NO: 93 - DNA, 94 bp, Artificial Sequence
  Primer H3.17
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
  misc_feature (46)..(46): n = 0.2G + 0.2A + 0.5T + 0.1C
  misc_feature (47)..(47): n = 0.4A + 0.2T + 0.4C
  misc_feature (48)..(48): n = 0.1G + 0.9C
  misc_feature (49)..(49): n = 0.2G + 0.2A + 0.5T + 0.1C
  misc_feature (50)..(50): n = 0.4A + 0.2T + 0.4C
  misc_feature (51)..(51): n = 0.1G + 0.9C
  misc_feature (52)..(52): n = 0.2G + 0.2A + 0.5T + 0.1C
  misc_feature (53)..(53): n = 0.4A + 0.2T + 0.4C
  misc_feature (54)..(54): n = 0.1G + 0.9C
  misc_feature (55)..(55): n = 0.2G + 0.2A + 0.5T + 0.1C
  misc_feature (56)..(56): n = 0.4A + 0.2T + 0.4C
  misc_feature (57)..(57): n = 0.1G + 0.9C
  misc_feature (58)..(58): n = 0.2G + 0.2A + 0.5T + 0.1C
  misc_feature (59)..(59): n = 0.4A + 0.2T + 0.4C
  misc_feature (60)..(60): n = 0.1G + 0.9C
  misc_feature (61)..(61): n = 0.2G + 0.2A + 0.5T + 0.1C
  misc_feature (62)..(62): n = 0.4A + 0.2T + 0.4C
  misc_feature (63)..(63): n = 0.1G + 0.9C
  misc_feature (64)..(64): n = 0.2G + 0.2A + 0.5T + 0.1C
  misc_feature (65)..(65): n = 0.4A + 0.2T + 0.4C
  misc_feature (66)..(66): n = 0.1G + 0.9C
  misc_feature (67)..(67): n = 0.2G + 0.2A + 0.5T + 0.1C
  misc_feature (68)..(68): n = 0.4A + 0.2T + 0.4C
  misc_feature (69)..(69): n = 0.1G + 0.9C
  misc_feature (70)..(70): n = 0.2G + 0.2A + 0.5T + 0.1C
  misc_feature (71)..(71): n = 0.4A + 0.2T + 0.4C
  misc_feature (72)..(72): n = 0.1G + 0.9C
  misc_feature (76)..(76): n is a, c, g, or t
gccgtctattatactgkccgcnnnnnnnnnnnnnnnnnnnnnnnnnnnnnnnnnnnnnnnnnnnnnnnnnnngstntkgactaccggggtcaag

SEQ ID NO: 94 - DNA, 97 bp, Artificial Sequence
  Primer H3.18
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
  misc_feature (46)..(46): n = 0.2G + 0.2A + 0.5T + 0.1C
  misc_feature (47)..(47): n = 0.4A + 0.2T + 0.4C
  misc_feature (48)..(48): n = 0.1G + 0.9C
  misc_feature (49)..(49): n = 0.2G + 0.2A + 0.5T + 0.1C
  misc_feature (50)..(50): n = 0.4A + 0.2T + 0.4C
  misc_feature (51)..(51): n = 0.1G + 0.9C
  misc_feature (52)..(52): n = 0.2G + 0.2A + 0.5T + 0.1C
  misc_feature (53)..(53): n = 0.4A + 0.2T + 0.4C
  misc_feature (54)..(54): n = 0.1G + 0.9C
  misc_feature (55)..(55): n = 0.2G + 0.2A + 0.5T + 0.1C
  misc_feature (56)..(56): n = 0.4A + 0.2T + 0.4C
  misc_feature (57)..(57): n = 0.1G + 0.9C
  misc_feature (58)..(58): n = 0.2G + 0.2A + 0.5T + 0.1C
  misc_feature (59)..(59): n = 0.4A + 0.2T + 0.4C
  misc_feature (60)..(60): n = 0.1G + 0.9C
  misc_feature (61)..(61): n = 0.2G + 0.2A + 0.5T + 0.1C
  misc_feature (62)..(62): n = 0.4A + 0.2T + 0.4C
  misc_feature (63)..(63): n = 0.1G + 0.9C
  misc_feature (64)..(64): n = 0.2G + 0.2A + 0.5T + 0.1C
  misc_feature (65)..(65): n = 0.4A + 0.2T + 0.4C
  misc_feature (66)..(66): n = 0.1G + 0.9C
  misc_feature (67)..(67): n = 0.2G + 0.2A + 0.5T + 0.1C
  misc_feature (68)..(68): n = 0.4A + 0.2T + 0.4C
  misc_feature (69)..(69): n = 0.1G + 0.9C
  misc_feature (70)..(70): n = 0.2G + 0.2A + 0.5T + 0.1C
  misc_feature (71)..(71): n = 0.4A + 0.2T + 0.4C
  misc_feature (72)..(72): n = 0.1G + 0.9C
  misc_feature (73)..(73): n = 0.2G + 0.2A + 0.5T + 0.1C
  misc_feature (74)..(74): n = 0.4A + 0.2T + 0.4C
  misc_feature (75)..(75): n = 0.1G + 0.9C
  misc_feature (79)..(79): n is a, c, g, or t
gccgtctattatactgkccgcnnnnnnnnnnnnnnnnnnnnnnnnnnnnnnnnnnnnnnnnnnnnnnnnnnnnnngstntkgactaccggggtcaag

SEQ ID NO: 95 - DNA, 100 bp, Artificial Sequence
  Primer H3.19
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
  misc_feature (46)..(46): n = 0.2G + 0.2A + 0.5T + 0.1C
  misc_feature (47)..(47): n = 0.4A + 0.2T + 0.4C
  misc_feature (48)..(48): n = 0.1G + 0.9C
  misc_feature (49)..(49): n = 0.2G + 0.2A + 0.5T + 0.1C
  misc_feature (50)..(50): n = 0.4A + 0.2T + 0.4C
  misc_feature (51)..(51): n = 0.1G + 0.9C
  misc_feature (52)..(52): n = 0.2G + 0.2A + 0.5T + 0.1C
  misc_feature (53)..(53): n = 0.4A + 0.2T + 0.4C
  misc_feature (54)..(54): n = 0.1G + 0.9C
  misc_feature (55)..(55): n = 0.2G + 0.2A + 0.5T + 0.1C
  misc_feature (56)..(56): n = 0.4A + 0.2T + 0.4C
  misc_feature (57)..(57): n = 0.1G + 0.9C
  misc_feature (58)..(58): n = 0.2G + 0.2A + 0.5T + 0.1C
  misc_feature (59)..(59): n = 0.4A + 0.2T + 0.4C
  misc_feature (60)..(60): n = 0.1G + 0.9C
  misc_feature (61)..(61): n = 0.2G + 0.2A + 0.5T + 0.1C
  misc_feature (62)..(62): n = 0.4A + 0.2T + 0.4C
  misc_feature (63)..(63): n = 0.1G + 0.9C
  misc_feature (64)..(64): n = 0.2G + 0.2A + 0.5T + 0.1C
  misc_feature (65)..(65): n = 0.4A + 0.2T + 0.4C
  misc_feature (66)..(66): n = 0.1G + 0.9C
  misc_feature (67)..(67): n = 0.2G + 0.2A + 0.5T + 0.1C
  misc_feature (68)..(68): n = 0.4A + 0.2T + 0.4C
  misc_feature (69)..(69): n = 0.1G + 0.9C
  misc_feature (70)..(70): n = 0.2G + 0.2A + 0.5T + 0.1C
  misc_feature (71)..(71): n = 0.4A + 0.2T + 0.4C
  misc_feature (72)..(72): n = 0.1G + 0.9C
  misc_feature (73)..(73): n = 0.2G + 0.2A + 0.5T + 0.1C
  misc_feature (74)..(74): n = 0.4A + 0.2T + 0.4C
  misc_feature (75)..(75): n = 0.1G + 0.9C
  misc_feature (76)..(76): n = 0.2G + 0.2A + 0.5T + 0.1C
  misc_feature (77)..(77): n = 0.4A + 0.2T + 0.4C
  misc_feature (78)..(78): n = 0.1G + 0.9C
  misc_feature (82)..(82): n is a, c, g, or t
gccgtctattatactgkccgcnnnnnnnnnnnnnnnnnnnnnnnnnnnnnnnnnnnnnnnnnnnnnnnnnnnnnnnnngstntkgactaccggggtcaag

SEQ ID NO: 96 - DNA, 103 bp, Artificial Sequence
  Primer H3.20
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
  misc_feature (46)..(46): n = 0.2G + 0.2A + 0.5T + 0.1C
  misc_feature (47)..(47): n = 0.4A + 0.2T + 0.4C
  misc_feature (48)..(48): n = 0.1G + 0.9C
  misc_feature (49)..(49): n = 0.2G + 0.2A + 0.5T + 0.1C
  misc_feature (50)..(50): n = 0.4A + 0.2T + 0.4C
  misc_feature (51)..(51): n = 0.1G + 0.9C
  misc_feature (52)..(52): n = 0.2G + 0.2A + 0.5T + 0.1C
  misc_feature (53)..(53): n = 0.4A + 0.2T + 0.4C
  misc_feature (54)..(54): n = 0.1G + 0.9C
  misc_feature (55)..(55): n = 0.2G + 0.2A + 0.5T + 0.1C
  misc_feature (56)..(56): n = 0.4A + 0.2T + 0.4C
  misc_feature (57)..(57): n = 0.1G + 0.9C
  misc_feature (58)..(58): n = 0.2G + 0.2A + 0.5T + 0.1C
  misc_feature (59)..(59): n = 0.4A + 0.2T + 0.4C
  misc_feature (60)..(60): n = 0.1G + 0.9C
  misc_feature (61)..(61): n = 0.2G + 0.2A + 0.5T + 0.1C
  misc_feature (62)..(62): n = 0.4A + 0.2T + 0.4C
  misc_feature (63)..(63): n = 0.1G + 0.9C
  misc_feature (64)..(64): n = 0.2G + 0.2A + 0.5T + 0.1C
  misc_feature (65)..(65): n = 0.4A + 0.2T + 0.4C
  misc_feature (66)..(66): n = 0.1G + 0.9C
  misc_feature (67)..(67): n = 0.2G + 0.2A + 0.5T + 0.1C
  misc_feature (68)..(68): n = 0.4A + 0.2T + 0.4C
  misc_feature (69)..(69): n = 0.1G + 0.9C
  misc_feature (70)..(70): n = 0.2G + 0.2A + 0.5T + 0.1C
  misc_feature (71)..(71): n = 0.4A + 0.2T + 0.4C
  misc_feature (72)..(72): n = 0.1G + 0.9C
  misc_feature (73)..(73): n = 0.2G + 0.2A + 0.5T + 0.1C
  misc_feature (74)..(74): n = 0.4A + 0.2T + 0.4C
  misc_feature (75)..(75): n = 0.1G + 0.9C
  misc_feature (76)..(76): n = 0.2G + 0.2A + 0.5T + 0.1C
  misc_feature (77)..(77): n = 0.4A + 0.2T + 0.4C
  misc_feature (78)..(78): n = 0.1G + 0.9C
  misc_feature (79)..(79): n = 0.2G + 0.2A + 0.5T + 0.1C
  misc_feature (80)..(80): n = 0.4A + 0.2T + 0.4C
  misc_feature (81)..(81): n = 0.1G + 0.9C
  misc_feature (85)..(85): n is a, c, g, or t
gccgtctattatactgkccgcnnnnnnnnnnnnnnnnnnnnnnnnnnnnnnnnnnnnnnnnnnnnnnnnnnnnnnnnnnnngstntkgactaccggggtcaag

SEQ ID NO: 97 - DNA, 57 bp, Artificial Sequence
  Primer H1
  misc_feature (21)..(21): n = 0.2G + 0.2A + 0.5T + 0.1C
  misc_feature (22)..(22): n = 0.4A + 0.2T + 0.4C
  misc_feature (23)..(23): n = 0.1G + 0.9C
  misc_feature (24)..(24): n is a, c, g, or t
  misc_feature (27)..(27): n = 0.2G + 0.2A + 0.5T + 0.1C
  misc_feature (28)..(28): n = 0.4A + 0.2T + 0.4C
  misc_feature (29)..(29): n = 0.1G + 0.9C
  misc_feature (30)..(30): n = 0.2G + 0.2A + 0.5T + 0.1C
  misc_feature (31)..(31): n = 0.4A + 0.2T + 0.4C
  misc_feature (32)..(32): n = 0.1G + 0.9C
  misc_feature (36)..(36): n = 0.2G + 0.2A + 0.5T + 0.1C
  misc_feature (37)..(37): n = 0.4A + 0.2T + 0.4C
  misc_feature (38)..(38): n = 0.1G + 0.9C
cctgtgcaatttctggcttcnnnnttnnnnnnactnnnatagactgggtgcgtcagg

SEQ ID NO: 98 - DNA, 61 bp, Artificial Sequence
  Primer H3.6
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (43)..(43): n is a, c, g, or t
gccgtctattattgtgkccgcnnnnnnnnnnnnnnnnnngstntkgactaccggggtcaag

SEQ ID NO: 99 - DNA, 64 bp, Artificial Sequence
  Primer H3.7
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n is a, c, g, or t
  misc_feature (42)..(42): n = 0.1G + 0.9C
  misc_feature (46)..(46): n is a, c, g, or t
gccgtctattattgtgkccgcnnnnnnnnnnnnnnnnnnnnngstntkgactaccggggtcaag

SEQ ID NO: 100 - DNA, 67 bp, Artificial Sequence
  Primer H3.8
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
  misc_feature (49)..(49): n is a, c, g, or t
gccgtctattattgtgkccgcnnnnnnnnnnnnnnnnnnnnnnnngstntkgactaccggggtcaag

SEQ ID NO: 101 - DNA, 70 bp, Artificial Sequence
  Primer H3.9
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
  misc_feature (46)..(46): n = 0.2G + 0.2A + 0.5T + 0.1C
  misc_feature (47)..(47): n = 0.4A + 0.2T + 0.4C
  misc_feature (48)..(48): n = 0.1G + 0.9C
  misc_feature (52)..(52): n is a, c, g, or t
gccgtctattattgtgkccgcnnnnnnnnnnnnnnnnnnnnnnnnnnngstntkgactaccggggtcaag

SEQ ID NO: 102 - DNA, 73 bp, Artificial Sequence
  Primer H3.10
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
  misc_feature (46)..(46): n = 0.2G + 0.2A + 0.5T + 0.1C
  misc_feature (47)..(47): n = 0.4A + 0.2T + 0.4C
  misc_feature (48)..(48): n = 0.1G + 0.9C
  misc_feature (49)..(49): n = 0.2G + 0.2A + 0.5T + 0.1C
  misc_feature (50)..(50): n = 0.4A + 0.2T + 0.4C
  misc_feature (51)..(51): n = 0.1G + 0.9C
  misc_feature (55)..(55): n is a, c, g, or t
gccgtctattattgtgkccgcnnnnnnnnnnnnnnnnnnnnnnnnnnnnnngstntkgactaccggggtcaag

SEQ ID NO: 103 - DNA, 76 bp, Artificial Sequence
  Primer H3.11
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
  misc_feature (46)..(46): n = 0.2G + 0.2A + 0.5T + 0.1C
  misc_feature (47)..(47): n = 0.4A + 0.2T + 0.4C
  misc_feature (48)..(48): n = 0.1G + 0.9C
  misc_feature (49)..(49): n = 0.2G + 0.2A + 0.5T + 0.1C
  misc_feature (50)..(50): n = 0.4A + 0.2T + 0.4C
  misc_feature (51)..(51): n = 0.1G + 0.9C
  misc_feature (52)..(52): n = 0.2G + 0.2A + 0.5T + 0.1C
  misc_feature (53)..(53): n = 0.4A + 0.2T + 0.4C
  misc_feature (54)..(54): n = 0.1G + 0.9C
  misc_feature (58)..(58): n is a, c, g, or t
gccgtctattattgtgkccgcnnnnnnnnnnnnnnnnnnnnnnnnnnnnnnnnngstntkgactaccggggtcaag

SEQ ID NO: 104 - DNA, 79 bp, Artificial Sequence
  Primer H3.12
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
  misc_feature (46)..(46): n = 0.2G + 0.2A + 0.5T + 0.1C
  misc_feature (47)..(47): n = 0.4A + 0.2T + 0.4C
  misc_feature (48)..(48): n = 0.1G + 0.9C
  misc_feature (49)..(49): n = 0.2G + 0.2A + 0.5T + 0.1C
  misc_feature (50)..(50): n = 0.4A + 0.2T + 0.4C
  misc_feature (51)..(51): n = 0.1G + 0.9C
  misc_feature (52)..(52): n = 0.2G + 0.2A + 0.5T + 0.1C
  misc_feature (53)..(53): n = 0.4A + 0.2T + 0.4C
  misc_feature (54)..(54): n = 0.1G + 0.9C
  misc_feature (55)..(55): n = 0.2G + 0.2A + 0.5T + 0.1C
  misc_feature (56)..(56): n = 0.4A + 0.2T + 0.4C
  misc_feature (57)..(57): n = 0.1G + 0.9C
  misc_feature (61)..(61): n is a, c, g, or t
gccgtctattattgtgkccgcnnnnnnnnnnnnnnnnnnnnnnnnnnnnnnnnnnnngstntkgactaccggggtcaag

SEQ ID NO: 105 - DNA, 82 bp, Artificial Sequence
  Primer H3.13
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
  misc_feature (46)..(46): n = 0.2G + 0.2A + 0.5T + 0.1C
  misc_feature (47)..(47): n = 0.4A + 0.2T + 0.4C
  misc_feature (48)..(48): n = 0.1G + 0.9C
  misc_feature (49)..(49): n = 0.2G + 0.2A + 0.5T + 0.1C
  misc_feature (50)..(50): n = 0.4A + 0.2T + 0.4C
  misc_feature (51)..(51): n = 0.1G + 0.9C
  misc_feature (52)..(52): n = 0.2G + 0.2A + 0.5T + 0.1C
  misc_feature (53)..(53): n = 0.4A + 0.2T + 0.4C
  misc_feature (54)..(54): n = 0.1G + 0.9C
  misc_feature (55)..(55): n = 0.2G + 0.2A + 0.5T + 0.1C
  misc_feature (56)..(56): n = 0.4A + 0.2T + 0.4C
  misc_feature (57)..(57): n = 0.1G + 0.9C
  misc_feature (58)..(58): n = 0.2G + 0.2A + 0.5T + 0.1C
  misc_feature (59)..(59): n = 0.4A + 0.2T + 0.4C
  misc_feature (60)..(60): n = 0.1G + 0.9C
  misc_feature (64)..(64): n is a, c, g, or t
gccgtctattattgtgkccgcnnnnnnnnnnnnnnnnnnnnnnnnnnnnnnnnnnnnnnngstntkgactaccggggtcaag

SEQ ID NO: 106 - DNA, 85 bp, Artificial Sequence
  Primer H3.14
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
  misc_feature (46)..(46): n = 0.2G + 0.2A + 0.5T + 0.1C
  misc_feature (47)..(47): n = 0.4A + 0.2T + 0.4C
  misc_feature (48)..(48): n = 0.1G + 0.9C
  misc_feature (49)..(49): n = 0.2G + 0.2A + 0.5T + 0.1C
  misc_feature (50)..(50): n = 0.4A + 0.2T + 0.4C
  misc_feature (51)..(51): n = 0.1G + 0.9C
  misc_feature (52)..(52): n = 0.2G + 0.2A + 0.5T + 0.1C
  misc_feature (53)..(53): n = 0.4A + 0.2T + 0.4C
  misc_feature (54)..(54): n = 0.1G + 0.9C
  misc_feature (55)..(55): n = 0.2G + 0.2A + 0.5T + 0.1C
  misc_feature (56)..(56): n = 0.4A + 0.2T + 0.4C
  misc_feature (57)..(57): n = 0.1G + 0.9C
  misc_feature (58)..(58): n = 0.2G + 0.2A + 0.5T + 0.1C
  misc_feature (59)..(59): n = 0.4A + 0.2T + 0.4C
  misc_feature (60)..(60): n = 0.1G + 0.9C
  misc_feature (61)..(61): n = 0.2G + 0.2A + 0.5T + 0.1C
  misc_feature (62)..(62): n = 0.4A + 0.2T + 0.4C
  misc_feature (63)..(63): n = 0.1G + 0.9C
  misc_feature (67)..(67): n is a, c, g, or t
gccgtctattattgtgkccgcnnnnnnnnnnnnnnnnnnnnnnnnnnnnnnnnnnnnnnnnnngstntkgactaccggggtcaag

SEQ ID NO: 107 - DNA, 88 bp, Artificial Sequence
  Primer H3.15
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
  misc_feature (46)..(46): n = 0.2G + 0.2A + 0.5T + 0.1C
  misc_feature (47)..(47): n = 0.4A + 0.2T + 0.4C
  misc_feature (48)..(48): n = 0.1G + 0.9C
  misc_feature (49)..(49): n = 0.2G + 0.2A + 0.5T + 0.1C
  misc_feature (50)..(50): n = 0.4A + 0.2T + 0.4C
  misc_feature (51)..(51): n = 0.1G + 0.9C
  misc_feature (52)..(52): n = 0.2G + 0.2A + 0.5T + 0.1C
  misc_feature (53)..(53): n = 0.4A + 0.2T + 0.4C
  misc_feature (54)..(54): n = 0.1G + 0.9C
  misc_feature (55)..(55): n = 0.2G + 0.2A + 0.5T + 0.1C
  misc_feature (56)..(56): n = 0.4A + 0.2T + 0.4C
  misc_feature (57)..(57): n = 0.1G + 0.9C
  misc_feature (58)..(58): n = 0.2G + 0.2A + 0.5T + 0.1C
  misc_feature (59)..(59): n = 0.4A + 0.2T + 0.4C
  misc_feature (60)..(60): n = 0.1G + 0.9C
  misc_feature (61)..(61): n = 0.2G + 0.2A + 0.5T + 0.1C
  misc_feature (62)..(62): n = 0.4A + 0.2T + 0.4C
  misc_feature (63)..(63): n = 0.1G + 0.9C
  misc_feature (64)..(64): n = 0.2G + 0.2A + 0.5T + 0.1C
  misc_feature (65)..(65): n = 0.4A + 0.2T + 0.4C
  misc_feature (66)..(66): n = 0.1G + 0.9C
  misc_feature (70)..(70): n is a, c, g, or t
gccgtctattattgtgkccgcnnnnnnnnnnnnnnnnnnnnnnnnnnnnnnnnnnnnnnnnnnnnngstntkgactaccggggtcaag

SEQ ID NO: 108 - DNA, 91 bp, Artificial Sequence
  Primer H3.16
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
  misc_feature (46)..(46): n = 0.2G + 0.2A + 0.5T + 0.1C
  misc_feature (47)..(47): n = 0.4A + 0.2T + 0.4C
  misc_feature (48)..(48): n = 0.1G + 0.9C
  misc_feature (49)..(49): n = 0.2G + 0.2A + 0.5T + 0.1C
  misc_feature (50)..(50): n = 0.4A + 0.2T + 0.4C
  misc_feature (51)..(51): n = 0.1G + 0.9C
  misc_feature (52)..(52): n = 0.2G + 0.2A + 0.5T + 0.1C
  misc_feature (53)..(53): n = 0.4A + 0.2T + 0.4C
  misc_feature (54)..(54): n = 0.1G + 0.9C
  misc_feature (55)..(55): n = 0.2G + 0.2A + 0.5T + 0.1C
  misc_feature (56)..(56): n = 0.4A + 0.2T + 0.4C
  misc_feature (57)..(57): n = 0.1G + 0.9C
  misc_feature (58)..(58): n = 0.2G + 0.2A + 0.5T + 0.1C
  misc_feature (59)..(59): n = 0.4A + 0.2T + 0.4C
  misc_feature (60)..(60): n = 0.1G + 0.9C
  misc_feature (61)..(61): n = 0.2G + 0.2A + 0.5T + 0.1C
  misc_feature (62)..(62): n = 0.4A + 0.2T + 0.4C
  misc_feature (63)..(63): n = 0.1G + 0.9C
  misc_feature (64)..(64): n = 0.2G + 0.2A + 0.5T + 0.1C
  misc_feature (65)..(65): n = 0.4A + 0.2T + 0.4C
  misc_feature (66)..(66): n = 0.1G + 0.9C
  misc_feature (67)..(67): n = 0.2G + 0.2A + 0.5T + 0.1C
  misc_feature (68)..(68): n = 0.4A + 0.2T + 0.4C
  misc_feature (69)..(69): n = 0.1G + 0.9C
  misc_feature (73)..(73): n is a, c, g, or t
gccgtctattattgtgkccgcnnnnnnnnnnnnnnnnnnnnnnnnnnnnnnnnnnnnnnnnnnnnnnnngstntkgactaccggggtcaag

SEQ ID NO: 109 - DNA, 94 bp, Artificial Sequence
  Primer H3.17
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
  misc_feature (46)..(46): n = 0.2G + 0.2A + 0.5T + 0.1C
  misc_feature (47)..(47): n = 0.4A + 0.2T + 0.4C
  misc_feature (48)..(48): n = 0.1G + 0.9C
  misc_feature (49)..(49): n = 0.2G + 0.2A + 0.5T + 0.1C
  misc_feature (50)..(50): n = 0.4A + 0.2T + 0.4C
  misc_feature (51)..(51): n = 0.1G + 0.9C
  misc_feature (52)..(52): n = 0.2G + 0.2A + 0.5T + 0.1C
  misc_feature (53)..(53): n = 0.4A + 0.2T + 0.4C
  misc_feature (54)..(54): n = 0.1G + 0.9C
  misc_feature (55)..(55): n = 0.2G + 0.2A + 0.5T + 0.1C
  misc_feature (56)..(56): n = 0.4A + 0.2T + 0.4C
  misc_feature (57)..(57): n = 0.1G + 0.9C
  misc_feature (58)..(58): n = 0.2G + 0.2A + 0.5T + 0.1C
  misc_feature (59)..(59): n = 0.4A + 0.2T + 0.4C
  misc_feature (60)..(60): n = 0.1G + 0.9C
  misc_feature (61)..(61): n = 0.2G + 0.2A + 0.5T + 0.1C
  misc_feature (62)..(62): n = 0.4A + 0.2T + 0.4C
  misc_feature (63)..(63): n = 0.1G + 0.9C
  misc_feature (63)..(63): n = 0.1G + 0.9C
  misc_feature (64)..(64): n = 0.2G + 0.2A + 0.5T + 0.1C
  misc_feature (65)..(65): n = 0.4A + 0.2T + 0.4C
  misc_feature (66)..(66): n = 0.1G + 0.9C
  misc_feature (67)..(67): n = 0.2G + 0.2A + 0.5T + 0.1C
  misc_feature (68)..(68): n = 0.4A + 0.2T + 0.4C
  misc_feature (69)..(69): n = 0.1G + 0.9C
  misc_feature (70)..(70): n = 0.2G + 0.2A + 0.5T + 0.1C
  misc_feature (71)..(71): n = 0.4A + 0.2T + 0.4C
  misc_feature (72)..(72): n is a, c, g, or t
  misc_feature (76)..(76): n is a, c, g, or t
gccgtctattattgtgkccgcnnnnnnnnnnnnnnnnnnnnnnnnnnnnnnnnnnnnnnnnnnnnnnnnnnngstntkgactaccggggtcaag

SEQ ID NO: 110 - DNA, 97 bp, Artificial Sequence
  Primer H3.18
  misc_feature (22)..(22): n = 0.2G + 0.2A + 0.5T + 0.1C
  misc_feature (23)..(23): n =  0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n =  0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n =  0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n =  0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n =  0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n =  0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n =  0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n =  0.4A + 0.2T + 0.4C
  misc_feature (45)..(45): n = 0.1G + 0.9C
  misc_feature (46)..(46): n = 0.2G + 0.2A + 0.5T + 0.1C
  misc_feature (47)..(47): n =  0.4A + 0.2T + 0.4C
  misc_feature (48)..(48): n = 0.1G + 0.9C
  misc_feature (49)..(49): n = 0.2G + 0.2A + 0.5T + 0.1C
  misc_feature (50)..(50): n =  0.4A + 0.2T + 0.4C
  misc_feature (51)..(51): n = 0.1G + 0.9C
  misc_feature (52)..(52): n = 0.2G + 0.2A + 0.5T + 0.1C
  misc_feature (53)..(53): n =  0.4A + 0.2T + 0.4C
  misc_feature (54)..(54): n = 0.1G + 0.9C
  misc_feature (55)..(55): n = 0.2G + 0.2A + 0.5T + 0.1C
  misc_feature (56)..(56): n =  0.4A + 0.2T + 0.4C
  misc_feature (57)..(57): n = 0.1G + 0.9C
  misc_feature (58)..(58): n = 0.2G + 0.2A + 0.5T + 0.1C
  misc_feature (59)..(59): n =  0.4A + 0.2T + 0.4C
  misc_feature (60)..(60): n = 0.1G + 0.9C
  misc_feature (60)..(60): n = 0.2G + 0.2A + 0.5T + 0.1C
  misc_feature (61)..(61): n =  0.4A + 0.2T + 0.4C
  misc_feature (62)..(62): n = 0.1G + 0.9C
  misc_feature (63)..(63): n is a, c, g, or t
  misc_feature (64)..(64): n = 0.2G + 0.2A + 0.5T + 0.1C
  misc_feature (65)..(65): n =  0.4A + 0.2T + 0.4C
  misc_feature (66)..(66): n = 0.1G + 0.9C
  misc_feature (67)..(67): n = 0.2G + 0.2A + 0.5T + 0.1C
  misc_feature (68)..(68): n =  0.4A + 0.2T + 0.4C
  misc_feature (69)..(69): n = 0.1G + 0.9C
  misc_feature (70)..(70): n = 0.2G + 0.2A + 0.5T + 0.1C
  misc_feature (71)..(71): n =  0.4A + 0.2T + 0.4C
  misc_feature (72)..(72): n = 0.1G + 0.9C
  misc_feature (73)..(73): n = 0.2G + 0.2A + 0.5T + 0.1C
  misc_feature (74)..(74): n =  0.4A + 0.2T + 0.4C
  misc_feature (75)..(75): n = 0.1G + 0.9C
  misc_feature (79)..(79): n is a, c, g, or t
gccgtctattattgtgkccgcnnnnnnnnnnnnnnnnnnnnnnnnnnnnnnnnnnnnnnnnnnnnnnnnnnnnnngstntkgactaccggggtcaag

SEQ ID NO: 111 - DNA, 100 bp, Artificial Sequence
  Primer H3.19
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
  misc_feature (46)..(46): n = 0.2G + 0.2A + 0.5T + 0.1C
  misc_feature (47)..(47): n = 0.4A + 0.2T + 0.4C
  misc_feature (48)..(48): n = 0.1G + 0.9C
  misc_feature (49)..(49): n = 0.2G + 0.2A + 0.5T + 0.1C
  misc_feature (50)..(50): n = 0.4A + 0.2T + 0.4C
  misc_feature (51)..(51): n = 0.1G + 0.9C
  misc_feature (52)..(52): n = 0.2G + 0.2A + 0.5T + 0.1C
  misc_feature (53)..(53): n = 0.4A + 0.2T + 0.4C
  misc_feature (54)..(54): n = 0.1G + 0.9C
  misc_feature (55)..(55): n = 0.2G + 0.2A + 0.5T + 0.1C
  misc_feature (56)..(56): n = 0.4A + 0.2T + 0.4C
  misc_feature (57)..(57): n = 0.1G + 0.9C
  misc_feature (58)..(58): n = 0.2G + 0.2A + 0.5T + 0.1C
  misc_feature (59)..(59): n = 0.4A + 0.2T + 0.4C
  misc_feature (60)..(60): n = 0.2G + 0.2A + 0.5T + 0.1C
  misc_feature (60)..(60): n = 0.1G + 0.9C
  misc_feature (61)..(61): n = 0.4A + 0.2T + 0.4C
  misc_feature (62)..(62): n = 0.1G + 0.9C
  misc_feature (63)..(63): n is a, c, g, or t
  misc_feature (64)..(64): n = 0.2G + 0.2A + 0.5T + 0.1C
  misc_feature (65)..(65): n = 0.4A + 0.2T + 0.4C
  misc_feature (66)..(66): n = 0.1G + 0.9C
  misc_feature (67)..(67): n = 0.2G + 0.2A + 0.5T + 0.1C
  misc_feature (68)..(68): n = 0.4A + 0.2T + 0.4C
  misc_feature (69)..(69): n = 0.1G + 0.9C
  misc_feature (70)..(70): n = 0.2G + 0.2A + 0.5T + 0.1C
  misc_feature (71)..(71): n = 0.4A + 0.2T + 0.4C
  misc_feature (72)..(72): n = 0.1G + 0.9C
  misc_feature (73)..(73): n = 0.2G + 0.2A + 0.5T + 0.1C
  misc_feature (74)..(74): n = 0.4A + 0.2T + 0.4C
  misc_feature (75)..(75): n = 0.1G + 0.9C
  misc_feature (76)..(76): n = 0.2G + 0.2A + 0.5T + 0.1C
  misc_feature (77)..(77): n = 0.4A + 0.2T + 0.4C
  misc_feature (78)..(78): n = 0.1G + 0.9C
  misc_feature (82)..(82): n is a, c, g, or t
gccgtctattattgtgkccgcnnnnnnnnnnnnnnnnnnnnnnnnnnnnnnnnnnnnnnnnnnnnnnnnnnnnnnnnngstntkgactaccggggtcaag

SEQ ID NO: 112 - DNA, 103 bp, Artificial Sequence
  Primer H3.20
  misc_feature (22)..(22): n = 0.2G + 0.2A + 0.5T + 0.1C
  misc_feature (23)..(23): n = 0.4A + 0.2T + 0.4C
  misc_feature (24)..(24): n = 0.1G + 0.9C
  misc_feature (25)..(25): n = 0.2G + 0.2A + 0.5T + 0.1C
  misc_feature (26)..(26): n = 0.4A + 0.2T + 0.4C
  misc_feature (27)..(27): n = 0.1G + 0.9C
  misc_feature (28)..(28): n = 0.2G + 0.2A + 0.5T + 0.1C
  misc_feature (29)..(29): n = 0.4A + 0.2T + 0.4C
  misc_feature (30)..(30): n = 0.1G + 0.9C
  misc_feature (31)..(31): n = 0.2G + 0.2A + 0.5T + 0.1C
  misc_feature (32)..(32): n = 0.4A + 0.2T + 0.4C
  misc_feature (33)..(33): n = 0.1G + 0.9C
  misc_feature (34)..(34): n = 0.2G + 0.2A + 0.5T + 0.1C
  misc_feature (35)..(35): n = 0.4A + 0.2T + 0.4C
  misc_feature (36)..(36): n = 0.1G + 0.9C
  misc_feature (37)..(37): n = 0.2G + 0.2A + 0.5T + 0.1C
  misc_feature (38)..(38): n = 0.4A + 0.2T + 0.4C
  misc_feature (39)..(39): n = 0.1G + 0.9C
  misc_feature (40)..(40): n = 0.2G + 0.2A + 0.5T + 0.1C
  misc_feature (41)..(41): n = 0.4A + 0.2T + 0.4C
  misc_feature (42)..(42): n = 0.1G + 0.9C
  misc_feature (43)..(43): n = 0.2G + 0.2A + 0.5T + 0.1C
  misc_feature (44)..(44): n = 0.4A + 0.2T + 0.4C
  misc_feature (45)..(45): n = 0.1G + 0.9C
  misc_feature (46)..(46): n = 0.2G + 0.2A + 0.5T + 0.1C
  misc_feature (47)..(47): n = 0.4A + 0.2T + 0.4C
  misc_feature (48)..(48): n = 0.1G + 0.9C
  misc_feature (49)..(49): n = 0.2G + 0.2A + 0.5T + 0.1C
  misc_feature (50)..(50): n = 0.4A + 0.2T + 0.4C
  misc_feature (51)..(51): n = 0.1G + 0.9C
  misc_feature (52)..(52): n = 0.2G + 0.2A + 0.5T + 0.1C
  misc_feature (53)..(53): n = 0.4A + 0.2T + 0.4C
  misc_feature (54)..(54): n = 0.1G + 0.9C
  misc_feature (55)..(55): n = 0.2G + 0.2A + 0.5T + 0.1C
  misc_feature (56)..(56): n = 0.4A + 0.2T + 0.4C
  misc_feature (57)..(57): n = 0.1G + 0.9C
  misc_feature (58)..(58): n = 0.2G + 0.2A + 0.5T + 0.1C
  misc_feature (59)..(59): n = 0.4A + 0.2T + 0.4C
  misc_feature (60)..(60): n = 0.2G + 0.2A + 0.5T + 0.1C
  misc_feature (60)..(60): n = 0.1G + 0.9C
  misc_feature (61)..(61): n = 0.4A + 0.2T + 0.4C
  misc_feature (62)..(62): n = 0.1G + 0.9C
  misc_feature (63)..(63): n is a, c, g, or t
  misc_feature (64)..(64): n = 0.2G + 0.2A + 0.5T + 0.1C
  misc_feature (65)..(65): n = 0.4A + 0.2T + 0.4C
  misc_feature (66)..(66): n = 0.1G + 0.9C
  misc_feature (67)..(67): n = 0.2G + 0.2A + 0.5T + 0.1C
  misc_feature (68)..(68): n = 0.4A + 0.2T + 0.4C
  misc_feature (69)..(69): n = 0.1G + 0.9C
  misc_feature (70)..(70): n = 0.2G + 0.2A + 0.5T + 0.1C
  misc_feature (71)..(71): n = 0.4A + 0.2T + 0.4C
  misc_feature (72)..(72): n = 0.1G + 0.9C
  misc_feature (73)..(73): n = 0.2G + 0.2A + 0.5T + 0.1C
  misc_feature (74)..(74): n = 0.4A + 0.2T + 0.4C
  misc_feature (75)..(75): n = 0.1G + 0.9C
  misc_feature (76)..(76): n = 0.2G + 0.2A + 0.5T + 0.1C
  misc_feature (77)..(77): n = 0.4A + 0.2T + 0.4C
  misc_feature (78)..(78): n = 0.1G + 0.9C
  misc_feature (79)..(79): n = 0.2G + 0.2A + 0.5T + 0.1C
  misc_feature (80)..(80): n = 0.4A + 0.2T + 0.4C
  misc_feature (81)..(81): n = 0.1G + 0.9C
  misc_feature (85)..(85): n is a, c, g, or t
gccgtctattattgtgkccgcnnnnnnnnnnnnnnnnnnnnnnnnnnnnnnnnnnnnnnnnnnnnnnnnnnnnnnnnnnnngstntkgactaccggggtcaag

SEQ ID NO: 113 - DNA, 54 bp, Artificial Sequence
  aVEGF-13A6 CDR1
  misc_feature (19)..(19): n= 70% T, 10% A, 10% G, 10% C
  misc_feature (20)..(20): n = 70% C, 10% A, 10% G, 10% T
  misc_feature (21)..(21): n= 70% T, 10% A, 10% G, 10% C
  misc_feature (22)..(22): n = 70% C, 10% A, 10% G, 10% T
  misc_feature (23)..(24): n= 70% T, 10% A, 10% G, 10% C
  misc_feature (25)..(25): n= 70% G, 10% A, 10% C, 10% T
  misc_feature (26)..(26): n = 70% C, 10% A, 10% G, 10% T
  misc_feature (27)..(27): n= 70% T, 10% A, 10% G, 10% C
  misc_feature (28)..(28): n= 70% G, 10% A, 10% C, 10% T
  misc_feature (29)..(29): n = 70% C, 10% A, 10% G, 10% T
  misc_feature (30)..(30): n= 70% T, 10% A, 10% G, 10% C
  misc_feature (31)..(31): n = 70% A, 10% G, 10% C, 10% T
  misc_feature (32)..(33): n = 70% C, 10% A, 10% G, 10% T
  misc_feature (34)..(34): n= 70% G, 10% A, 10% C, 10% T
  misc_feature (35)..(35): n = 70% A, 10% G, 10% C, 10% T
  misc_feature (36)..(36): n = 70% C, 10% A, 10% G, 10% T
tgtgcaatttctggcttcnnnnnnnnnnnnnnnnnnatagactgggtgcgtcag

SEQ ID NO: 114 - DNA, 60 bp, Artificial Sequence
  aVEGF-13A6 CDR2
  misc_feature (19)..(21): n = 70% T, 10% A, 10% G, 10% C
  misc_feature (22)..(23): n = 70% C, 10% A, 10% G, 10% T
  misc_feature (24)..(24): n = 70% G, 10% A, 10% C, 10% T
  misc_feature (25)..(25): n = 70% T, 10% A, 10% G, 10% C
  misc_feature (26)..(26): n = 70% C, 10% A, 10% G, 10% T
  misc_feature (27)..(27): n = 70% T, 10% A, 10% G, 10% C
  misc_feature (28)..(28): n = 70% G, 10% A, 10% C, 10% T
  misc_feature (29)..(29): n = 70% C, 10% A, 10% G, 10% T
  misc_feature (30)..(30): n = 70% T, 10% A, 10% G, 10% C
  misc_feature (31)..(32): n = 70% G, 10% A, 10% C, 10% T
  misc_feature (33)..(34): n = 70% T, 10% A, 10% G, 10% C
  misc_feature (35)..(35): n = 70% C, 10% A, 10% G, 10% T
  misc_feature (36)..(36): n = 70% T, 10% A, 10% G, 10% C
  misc_feature (37)..(37): n = 70% A, 10% G, 10% C, 10% T
  misc_feature (38)..(38): n = 70% C, 10% A, 10% G, 10% T
  misc_feature (39)..(39): n = 70% C, 10% A, 10% G, 10% T
  misc_feature (40)..(40): n = 70% G, 10% A, 10% C, 10% T
  misc_feature (41)..(41): n = 70% A, 10% G, 10% C, 10% T
  misc_feature (42)..(42): n = 70% C, 10% A, 10% G, 10% T
gaatgggttgcaaggattnnnnnnnnnnnnnnnnnnnnnnnntatgccgatagcgtcaag

SEQ ID NO: 115 - DNA, 99 bp, Artificial Sequence
  aVEGF-13A6 CDR3
  misc_feature (19)..(20): n = 70% G, 10% A, 10% C, 10% T
  misc_feature (21)..(21): n = 70% T, 10% A, 10% G, 10% C
  misc_feature (22)..(22): n = 70% A, 10% G, 10% C, 10% T
  misc_feature (23)..(23): n = 70% G, 10% A, 10% C, 10% T
  misc_feature (24)..(24): n = 70% A, 10% G, 10% C, 10% T
  misc_feature (25)..(25): n = 70% T, 10% A, 10% G, 10% C
  misc_feature (26)..(26): n = 70% C, 10% A, 10% G, 10% T
  misc_feature (27)..(27): n = 70% T, 10% A, 10% G, 10% C
  misc_feature (28)..(28): n = 70% G, 10% A, 10% C, 10% T
  misc_feature (29)..(29): n = 70% A, 10% G, 10% C, 10% T
  misc_feature (30)..(30): n = 70% C, 10% A, 10% G, 10% T
  misc_feature (31)..(31): n = 70% T, 10% A, 10% G, 10% C
  misc_feature (32)..(32): n = 70% C, 10% A, 10% G, 10% T
  misc_feature (33)..(34): n = 70% T, 10% A, 10% G, 10% C
  misc_feature (35)..(35): n = 70% A, 10% G, 10% C, 10% T
  misc_feature (36)..(36): n = 70% C, 10% A, 10% G, 10% T
  misc_feature (37)..(37): n = 70% T, 10% A, 10% G, 10% C
  misc_feature (38)..(38): n = 70% C, 10% A, 10% G, 10% T
  misc_feature (39)..(40): n = 70% T, 10% A, 10% G, 10% C
  misc_feature (41)..(41): n = 70% A, 10% G, 10% C, 10% T
  misc_feature (42)..(42): n = 70% C, 10% A, 10% G, 10% T
  misc_feature (43)..(44): n = 70% A, 10% G, 10% C, 10% T
  misc_feature (45)..(45): n = 70% T, 10% A, 10% G, 10% C
  misc_feature (46)..(46): n = 70% G, 10% A, 10% C, 10% T
  misc_feature (47)..(51): n = 70% T, 10% A, 10% G, 10% C
  misc_feature (52)..(52): n = 70% C, 10% A, 10% G, 10% T
  misc_feature (53)..(54): n = 70% T, 10% A, 10% G, 10% C
  misc_feature (55)..(55): n = 70% C, 10% A, 10% G, 10% T
  misc_feature (56)..(56): n = 70% A, 10% G, 10% C, 10% T
  misc_feature (57)..(57): n = 70% C, 10% A, 10% G, 10% T
  misc_feature (58)..(59): n = 70% A, 10% G, 10% C, 10% T
  misc_feature (60)..(60): n = 70% T, 10% A, 10% G, 10% C
  misc_feature (61)..(62): n = 70% C, 10% A, 10% G, 10% T
  misc_feature (63)..(63): n = 70% G, 10% A, 10% C, 10% T
  misc_feature (64)..(64): n = 70% A, 10% G, 10% C, 10% T
  misc_feature (65)..(66): n = 70% C, 10% A, 10% G, 10% T
  misc_feature (67)..(67): n = 70% T, 10% A, 10% G, 10% C
  misc_feature (68)..(68): n = 70% A, 10% G, 10% C, 10% T
  misc_feature (69)..(69): n = 70% C, 10% A, 10% G, 10% T
  misc_feature (70)..(70): n = 70% A, 10% G, 10% C, 10% T
  misc_feature (71)..(72): n = 70% C, 10% A, 10% G, 10% T
  misc_feature (73)..(73): n = 70% G, 10% A, 10% C, 10% T
  misc_feature (74)..(74): n = 70% C, 10% A, 10% G, 10% T
  misc_feature (75)..(75): n = 70% T, 10% A, 10% G, 10% C
  misc_feature (76)..(76): n = 70% C, 10% A, 10% G, 10% T
  misc_feature (77)..(78): n = 70% T, 10% A, 10% G, 10% C
  misc_feature (79)..(79): n = 70% G, 10% A, 10% C, 10% T
  misc_feature (80)..(80): n = 70% A, 10% G, 10% C, 10% T
  misc_feature (81)..(81): n = 70% C, 10% A, 10% G, 10% T
actgccgtctattattgtnnnnnnnnnnnnnnnnnnnnnnnnnnnnnnnnnnnnnnnnnnnnnnnnnnnnnnnnnnnnnnntaccggggtcaaggaaca

SEQ ID NO: 116 - protein, 130 aa, Artificial Sequence
  VEGFA-binding single domain antibody 16A2.1 (AST)
SEVQLVESGGGLVQPGGSLRLSCAISGFALAETDIDWVRQAPGKGLEWVARIFSSGGNTDYADSVKGRFTISADTSKNTVYLQMNSLRAEDTAVYYCGRSDSIAYNVILPASTYTARDYRGQGTLVTVSS

SEQ ID NO: 117 - protein, 130 aa, Artificial Sequence
  VEGFA-binding single domain antibody 16A2.1 (NSA)
SEVQLVESGGGLVQPGGSLRLSCAISGFALAETDIDWVRQAPGKGLEWVARIFSSGGNTDYADSVKGRFTISADTSKNTVYLQMNSLRAEDTAVYYCGRSDSIAYNVILPNSAYTARDYRGQGTLVTVSS

SEQ ID NO: 118 - DNA, 54 bp, Artificial Sequence; identical to SEQ ID NO: 113
  aVEGF-16A2.1 CDR1
  misc_feature (19)..(19): n = 70% G, 10% A, 10% C, 10% T
  misc_feature (20)..(20): n = 70% C, 10% A, 10% G, 10% T
  misc_feature (21)..(21): n = 70% T, 10% A, 10% G, 10% C
  misc_feature (22)..(22): n = 70% C, 10% A, 10% G, 10% T
  misc_feature (23)..(24): n = 70% T, 10% A, 10% G, 10% C
  misc_feature (25)..(25): n = 70% G, 10% A, 10% C, 10% T
  misc_feature (26)..(26): n = 70% C, 10% A, 10% G, 10% T
  misc_feature (27)..(27): n = 70% T, 10% A, 10% G, 10% C
  misc_feature (28)..(28): n = 70% G, 10% A, 10% C, 10% T
  misc_feature (29)..(31): n is a, c, g, or t
  misc_feature (32)..(33): n = 70% C, 10% A, 10% G, 10% T
  misc_feature (34)..(34): n = 70% G, 10% A, 10% C, 10% T
  misc_feature (35)..(35): n = 70% A, 10% G, 10% C, 10% T
  misc_feature (36)..(36): n = 70% C, 10% A, 10% G, 10% T

SEQ ID NO: 119 - DNA, 60 bp, Artificial Sequence; identical to SEQ ID NO: 114
  aVEGF-16A2.1 CDR2
  misc_feature (19)..(22): n = 70% T, 10% A, 10% G, 10% C
  misc_feature (23)..(23): n = 70% C, 10% A, 10% G, 10% T
  misc_feature (24)..(25): n = 70% T, 10% A, 10% G, 10% C
  misc_feature (26)..(26): n = 70% C, 10% A, 10% G, 10% T
  misc_feature (27)..(27): n = 70% T, 10% A, 10% G, 10% C
  misc_feature (28)..(29): n = 70% G, 10% A, 10% C, 10% T
  misc_feature (30)..(30): n = 70% T, 10% A, 10% G, 10% C
  misc_feature (31)..(32): n = 70% G, 10% A, 10% C, 10% T
  misc_feature (33)..(33): n = 70% T, 10% A, 10% G, 10% C
  misc_feature (34)..(35): n = 70% A, 10% G, 10% C, 10% T
  misc_feature (36)..(36): n = 70% T, 10% A, 10% G, 10% C
  misc_feature (37)..(37): n = 70% A, 10% G, 10% C, 10% T
  misc_feature (38)..(39): n = 70% C, 10% A, 10% G, 10% T
  misc_feature (40)..(40): n = 70% G, 10% A, 10% C, 10% T
  misc_feature (41)..(41): n = 70% A, 10% G, 10% C, 10% T
  misc_feature (42)..(42): n = 70% C, 10% A, 10% G, 10% T

SEQ ID NO: 120 - DNA, 99 bp, Artificial Sequence; identical to SEQ ID NO: 115
  aVEGF-16A2.1 CDR3
  misc_feature (19)..(20): n = 70% G, 10% A, 10% C, 10% T
  misc_feature (21)..(21): n = 70% T, 10% A, 10% G, 10% C
  misc_feature (22)..(22): n = 70% A, 10% G, 10% C, 10% T
  misc_feature (23)..(23): n = 70% G, 10% A, 10% C, 10% T
  misc_feature (24)..(24): n = 70% A, 10% G, 10% C, 10% T
  misc_feature (25)..(25): n = 70% T, 10% A, 10% G, 10% C
  misc_feature (26)..(26): n = 70% C, 10% A, 10% G, 10% T
  misc_feature (27)..(27): n = 70% T, 10% A, 10% G, 10% C
  misc_feature (28)..(28): n = 70% G, 10% A, 10% C, 10% T
  misc_feature (29)..(29): n = 70% A, 10% G, 10% C, 10% T
  misc_feature (30)..(30): n = 70% C, 10% A, 10% G, 10% T
  misc_feature (31)..(31): n = 70% T, 10% A, 10% G, 10% C
  misc_feature (32)..(32): n = 70% C, 10% A, 10% G, 10% T
  misc_feature (33)..(33): n = 70% T, 10% A, 10% G, 10% C
  misc_feature (34)..(34): n = 70% A, 10% G, 10% C, 10% T
  misc_feature (35)..(36): n = 70% T, 10% A, 10% G, 10% C
  misc_feature (37)..(37): n = 70% G, 10% A, 10% C, 10% T
  misc_feature (38)..(38): n = 70% C, 10% A, 10% G, 10% T
  misc_feature (39)..(40): n = 70% T, 10% A, 10% G, 10% C
  misc_feature (41)..(41): n = 70% A, 10% G, 10% C, 10% T
  misc_feature (42)..(42): n = 70% C, 10% A, 10% G, 10% T
  misc_feature (43)..(44): n = 70% A, 10% G, 10% C, 10% T
  misc_feature (45)..(45): n = 70% T, 10% A, 10% G, 10% C
  misc_feature (46)..(46): n = 70% G, 10% A, 10% C, 10% T
  misc_feature (47)..(48): n = 70% T, 10% A, 10% G, 10% C
  misc_feature (49)..(49): n = 70% A, 10% G, 10% C, 10% T
  misc_feature (50)..(51): n = 70% T, 10% A, 10% G, 10% C
  misc_feature (52)..(52): n = 70% C, 10% A, 10% G, 10% T
  misc_feature (53)..(54): n = 70% T, 10% A, 10% G, 10% C
  misc_feature (55)..(56): n = 70% C, 10% A, 10% G, 10% T
  misc_feature (57)..(57): n = 70% G, 10% A, 10% C, 10% T
  misc_feature (58)..(59): n = 70% A, 10% G, 10% C, 10% T
  misc_feature (60)..(61): n = 70% T, 10% A, 10% G, 10% C
  misc_feature (62)..(62): n = 70% T, 10% A, 10% G, 10% C
  misc_feature (62)..(62): n = 70% C, 10% A, 10% G, 10% T
  misc_feature (63)..(63): n is a, c, g, or t
  misc_feature (64)..(64): n = 70% A, 10% G, 10% C, 10% T
  misc_feature (65)..(66): n = 70% C, 10% A, 10% G, 10% T
  misc_feature (67)..(67): n = 70% T, 10% A, 10% G, 10% C
  misc_feature (68)..(68): n = 70% A, 10% G, 10% C, 10% T
  misc_feature (69)..(69): n = 70% C, 10% A, 10% G, 10% T
  misc_feature (70)..(70): n = 70% A, 10% G, 10% C, 10% T
  misc_feature (71)..(72): n = 70% C, 10% A, 10% G, 10% T
  misc_feature (73)..(73): n = 70% G, 10% A, 10% C, 10% T
  misc_feature (74)..(74): n = 70% C, 10% A, 10% G, 10% T
  misc_feature (75)..(75): n = 70% T, 10% A, 10% G, 10% C
  misc_feature (76)..(76): n = 70% A, 10% G, 10% C, 10% T
  misc_feature (77)..(77): n = 70% G, 10% A, 10% C, 10% T
  misc_feature (78)..(78): n = 70% A, 10% G, 10% C, 10% T
  misc_feature (79)..(79): n = 70% G, 10% A, 10% C, 10% T
  misc_feature (80)..(80): n = 70% A, 10% G, 10% C, 10% T
  misc_feature (81)..(81): n = 70% C, 10% A, 10% G, 10% T
  misc_feature (81)..(81): n = 70% C, 10% A, 10% G, 10% T

SEQ ID NO: 121 - DNA, 99 bp, Artificial Sequence; identical to SEQ ID NO: 115
  aVEGF-16A2.1(AST) CDR3
  misc_feature (19)..(20): n = 70% G, 10% A, 10% C, 10% T
  misc_feature (21)..(21): n = 70% T, 10% A, 10% G, 10% C
  misc_feature (22)..(22): n = 70% A, 10% G, 10% C, 10% T
  misc_feature (23)..(23): n = 70% G, 10% A, 10% C, 10% T
  misc_feature (24)..(24): n = 70% A, 10% G, 10% C, 10% T
  misc_feature (25)..(25): n = 70% T, 10% A, 10% G, 10% C
  misc_feature (26)..(26): n = 70% C, 10% A, 10% G, 10% T
  misc_feature (27)..(27): n = 70% T, 10% A, 10% G, 10% C
  misc_feature (28)..(28): n = 70% G, 10% A, 10% C, 10% T
  misc_feature (29)..(29): n = 70% A, 10% G, 10% C, 10% T
  misc_feature (30)..(30): n = 70% C, 10% A, 10% G, 10% T
  misc_feature (31)..(31): n = 70% T, 10% A, 10% G, 10% C
  misc_feature (32)..(32): n = 70% C, 10% A, 10% G, 10% T
  misc_feature (33)..(33): n = 70% T, 10% A, 10% G, 10% C
  misc_feature (34)..(34): n = 70% A, 10% G, 10% C, 10% T
  misc_feature (35)..(36): n = 70% T, 10% A, 10% G, 10% C
  misc_feature (37)..(37): n = 70% G, 10% A, 10% C, 10% T
  misc_feature (38)..(38): n = 70% C, 10% A, 10% G, 10% T
  misc_feature (39)..(40): n = 70% T, 10% A, 10% G, 10% C
  misc_feature (41)..(41): n = 70% A, 10% G, 10% C, 10% T
  misc_feature (42)..(42): n = 70% C, 10% A, 10% G, 10% T
  misc_feature (43)..(44): n = 70% A, 10% G, 10% C, 10% T
  misc_feature (45)..(45): n = 70% T, 10% A, 10% G, 10% C
  misc_feature (46)..(46): n = 70% G, 10% A, 10% C, 10% T
  misc_feature (47)..(48): n = 70% T, 10% A, 10% G, 10% C
  misc_feature (49)..(49): n = 70% A, 10% G, 10% C, 10% T
  misc_feature (50)..(51): n = 70% T, 10% A, 10% G, 10% C
  misc_feature (52)..(52): n = 70% C, 10% A, 10% G, 10% T
  misc_feature (53)..(54): n = 70% T, 10% A, 10% G, 10% C
  misc_feature (55)..(56): n = 70% C, 10% A, 10% G, 10% T
  misc_feature (57)..(57): n = 70% G, 10% A, 10% C, 10% T
  misc_feature (58)..(59): n = 70% A, 10% G, 10% C, 10% T
  misc_feature (60)..(61): n = 70% T, 10% A, 10% G, 10% C
  misc_feature (62)..(62): n = 70% C, 10% A, 10% G, 10% T
  misc_feature (63)..(63): n = 70% T, 10% A, 10% G, 10% C
  misc_feature (64)..(64): n = 70% G, 10% A, 10% C, 10% T
  misc_feature (65)..(65): n = 70% C, 10% A, 10% G, 10% T
  misc_feature (66)..(67): n = 70% T, 10% A, 10% G, 10% C
  misc_feature (68)..(68): n = 70% A, 10% G, 10% C, 10% T
  misc_feature (69)..(69): n = 70% C, 10% A, 10% G, 10% T
  misc_feature (70)..(70): n = 70% A, 10% G, 10% C, 10% T
  misc_feature (71)..(72): n = 70% C, 10% A, 10% G, 10% T
  misc_feature (73)..(73): n = 70% G, 10% A, 10% C, 10% T
  misc_feature (74)..(74): n = 70% C, 10% A, 10% G, 10% T
  misc_feature (75)..(75): n = 70% T, 10% A, 10% G, 10% C
  misc_feature (76)..(76): n is a, c, g, or t
  misc_feature (77)..(77): n = 70% G, 10% A, 10% C, 10% T
  misc_feature (78)..(78): n = 70% A, 10% G, 10% C, 10% T
  misc_feature (79)..(79): n = 70% G, 10% A, 10% C, 10% T
  misc_feature (80)..(80): n = 70% A, 10% G, 10% C, 10% T
  misc_feature (81)..(81): n = 70% C, 10% A, 10% G, 10% T

SEQ ID NO: 122 - DNA, 99 bp, Artificial Sequence; identical to SEQ ID NO: 115
  aVEGF-16A2.1(NSA) CDR3
  misc_feature (19)..(20): n = 70% G, 10% A, 10% C, 10% T
  misc_feature (21)..(21): n = 70% T, 10% A, 10% G, 10% C
  misc_feature (22)..(22): n = 70% A, 10% G, 10% C, 10% T
  misc_feature (23)..(23): n = 70% G, 10% A, 10% C, 10% T
  misc_feature (24)..(24): n = 70% A, 10% G, 10% C, 10% T
  misc_feature (25)..(25): n = 70% T, 10% A, 10% G, 10% C
  misc_feature (26)..(26): n = 70% C, 10% A, 10% G, 10% T
  misc_feature (27)..(27): n = 70% T, 10% A, 10% G, 10% C
  misc_feature (28)..(28): n = 70% G, 10% A, 10% C, 10% T
  misc_feature (29)..(29): n = 70% A, 10% G, 10% C, 10% T
  misc_feature (30)..(30): n = 70% C, 10% A, 10% G, 10% T
  misc_feature (31)..(31): n = 70% T, 10% A, 10% G, 10% C
  misc_feature (32)..(32): n = 70% C, 10% A, 10% G, 10% T
  misc_feature (33)..(33): n = 70% T, 10% A, 10% G, 10% C
  misc_feature (34)..(34): n = 70% A, 10% G, 10% C, 10% T
  misc_feature (35)..(36): n = 70% T, 10% A, 10% G, 10% C
  misc_feature (37)..(37): n = 70% G, 10% A, 10% C, 10% T
  misc_feature (38)..(38): n = 70% C, 10% A, 10% G, 10% T
  misc_feature (39)..(40): n = 70% T, 10% A, 10% G, 10% C
  misc_feature (41)..(41): n = 70% A, 10% G, 10% C, 10% T
  misc_feature (42)..(42): n = 70% C, 10% A, 10% G, 10% T
  misc_feature (43)..(44): n = 70% A, 10% G, 10% C, 10% T
  misc_feature (45)..(45): n = 70% T, 10% A, 10% G, 10% C
  misc_feature (46)..(46): n = 70% G, 10% A, 10% C, 10% T
  misc_feature (47)..(48): n = 70% T, 10% A, 10% G, 10% C
  misc_feature (49)..(49): n = 70% A, 10% G, 10% C, 10% T
  misc_feature (50)..(51): n = 70% T, 10% A, 10% G, 10% C
  misc_feature (52)..(52): n = 70% C, 10% A, 10% G, 10% T
  misc_feature (53)..(54): n = 70% T, 10% A, 10% G, 10% C
  misc_feature (55)..(56): n = 70% C, 10% A, 10% G, 10% T
  misc_feature (57)..(58): n = 70% G, 10% A, 10% C, 10% T
  misc_feature (59)..(59): n = 70% C, 10% A, 10% G, 10% T
  misc_feature (60)..(61): n = 70% T, 10% A, 10% G, 10% C
  misc_feature (62)..(62): n = 70% C, 10% A, 10% G, 10% T
  misc_feature (63)..(63): n = 70% T, 10% A, 10% G, 10% C
  misc_feature (64)..(64): n = 70% A, 10% G, 10% C, 10% T
  misc_feature (65)..(66): n = 70% C, 10% A, 10% G, 10% T
  misc_feature (67)..(67): n = 70% T, 10% A, 10% G, 10% C
  misc_feature (68)..(68): n = 70% A, 10% G, 10% C, 10% T
  misc_feature (69)..(69): n = 70% C, 10% A, 10% G, 10% T
  misc_feature (70)..(70): n = 70% A, 10% G, 10% C, 10% T
  misc_feature (71)..(72): n = 70% C, 10% A, 10% G, 10% T
  misc_feature (73)..(73): n = 70% G, 10% A, 10% C, 10% T
  misc_feature (74)..(74): n = 70% C, 10% A, 10% G, 10% T
  misc_feature (75)..(75): n = 70% T, 10% A, 10% G, 10% C
  misc_feature (76)..(76): n = 70% A, 10% G, 10% C, 10% T
  misc_feature (77)..(77): n = 70% G, 10% A, 10% C, 10% T
  misc_feature (78)..(78): n = 70% A, 10% G, 10% C, 10% T
  misc_feature (79)..(79): n = 70% G, 10% A, 10% C, 10% T
  misc_feature (80)..(80): n = 70% A, 10% G, 10% C, 10% T
  misc_feature (81)..(81): n = 70% C, 10% A, 10% G, 10% T

SEQ ID NO: 123 - DNA, 54 bp, Artificial Sequence; identical to SEQ ID NO: 113
  aVEGF-16C2.1 CDR1
  misc_feature (19)..(19): n = 70% T, 10% A, 10% G, 10% C
  misc_feature (20)..(20): n = 70% C, 10% A, 10% G, 10% T
  misc_feature (21)..(21): n = 70% T, 10% A, 10% G, 10% C
  misc_feature (22)..(22): n = 70% C, 10% A, 10% G, 10% T
  misc_feature (23)..(24): n = 70% T, 10% A, 10% G, 10% C
  misc_feature (25)..(25): n = 70% G, 10% A, 10% C, 10% T
  misc_feature (26)..(26): n = 70% C, 10% A, 10% G, 10% T
  misc_feature (27)..(27): n = 70% T, 10% A, 10% G, 10% C
  misc_feature (28)..(28): n = 70% G, 10% A, 10% C, 10% T
  misc_feature (29)..(29): n = 70% C, 10% A, 10% G, 10% T
  misc_feature (30)..(30): n = 70% T, 10% A, 10% G, 10% C
  misc_feature (31)..(31): n = 70% A, 10% G, 10% C, 10% T
  misc_feature (32)..(33): n = 70% C, 10% A, 10% G, 10% T
  misc_feature (34)..(34): n = 70% G, 10% A, 10% C, 10% T
  misc_feature (35)..(35): n = 70% A, 10% G, 10% C, 10% T
  misc_feature (36)..(36): n = 70% C, 10% A, 10% G, 10% T

SEQ ID NO: 124 - DNA, 60 bp, Artificial Sequence; identical to SEQ ID NO: 114
  aVEGF-16C2.1 CDR2
  misc_feature (19)..(22): n = 70% T, 10% A, 10% G, 10% C
  misc_feature (23)..(23): n = 70% C, 10% A, 10% G, 10% T
  misc_feature (24)..(24): n = 70% T, 10% A, 10% G, 10% C
  misc_feature (25)..(26): n = 70% C, 10% A, 10% G, 10% T
  misc_feature (27)..(27): n = 70% G, 10% A, 10% C, 10% T
  misc_feature (28)..(28): n = 70% T, 10% A, 10% G, 10% C
  misc_feature (29)..(29): n = 70% C, 10% A, 10% G, 10% T
  misc_feature (30)..(30): n = 70% T, 10% A, 10% G, 10% C
  misc_feature (31)..(31): n = 70% G, 10% A, 10% C, 10% T
  misc_feature (32)..(32): n = 70% A, 10% G, 10% C, 10% T
  misc_feature (33)..(33): n = 70% C, 10% A, 10% G, 10% T
  misc_feature (34)..(36): n = 70% T, 10% A, 10% G, 10% C
  misc_feature (37)..(37): n = 70% A, 10% G, 10% C, 10% T
  misc_feature (38)..(39): n = 70% C, 10% A, 10% G, 10% T
  misc_feature (40)..(40): n = 70% G, 10% A, 10% C, 10% T
  misc_feature (41)..(41): n = 70% A, 10% G, 10% C, 10% T
  misc_feature (42)..(42): n = 70% C, 10% A, 10% G, 10% T

SEQ ID NO: 125 - DNA, 99 bp, Artificial Sequence; identical to SEQ ID NO: 115
  aVEGF-16C2.1 CDR3
  misc_feature (19)..(20): n = 70% G, 10% A, 10% C, 10% T
  misc_feature (21)..(21): n = 70% T, 10% A, 10% G, 10% C
  misc_feature (22)..(22): n = 70% A, 10% G, 10% C, 10% T
  misc_feature (23)..(23): n = 70% G, 10% A, 10% C, 10% T
  misc_feature (24)..(24): n = 70% A, 10% G, 10% C, 10% T
  misc_feature (25)..(26): n = 70% C, 10% A, 10% G, 10% T
  misc_feature (27)..(28): n = 70% G, 10% A, 10% C, 10% T
  misc_feature (29)..(29): n = 70% A, 10% G, 10% C, 10% T
  misc_feature (30)..(30): n = 70% C, 10% A, 10% G, 10% T
  misc_feature (31)..(31): n = 70% A, 10% G, 10% C, 10% T
  misc_feature (32)..(33): n = 70% C, 10% A, 10% G, 10% T
  misc_feature (34)..(34): n = 70% T, 10% A, 10% G, 10% C
  misc_feature (35)..(35): n = 70% A, 10% G, 10% C, 10% T
  misc_feature (36)..(36): n = 70% C, 10% A, 10% G, 10% T
  misc_feature (37)..(37): n = 70% G, 10% A, 10% C, 10% T
  misc_feature (38)..(38): n = 70% C, 10% A, 10% G, 10% T
  misc_feature (39)..(40): n = 70% T, 10% A, 10% G, 10% C
  misc_feature (41)..(41): n = 70% A, 10% G, 10% C, 10% T
  misc_feature (42)..(42): n = 70% C, 10% A, 10% G, 10% T
  misc_feature (43)..(44): n = 70% A, 10% G, 10% C, 10% T
  misc_feature (45)..(45): n is a, c, g, or t
  misc_feature (46)..(46): n = 70% A, 10% G, 10% C, 10% T
  misc_feature (47)..(51): n = 70% T, 10% A, 10% G, 10% C
  misc_feature (52)..(52): n = 70% C, 10% A, 10% G, 10% T
  misc_feature (53)..(54): n = 70% T, 10% A, 10% G, 10% C
  misc_feature (55)..(55): n = 70% G, 10% A, 10% C, 10% T
  misc_feature (56)..(56): n = 70% A, 10% G, 10% C, 10% T
  misc_feature (57)..(57): n = 70% C, 10% A, 10% G, 10% T
  misc_feature (58)..(58): n = 70% A, 10% G, 10% C, 10% T
  misc_feature (59)..(62): n = 70% C, 10% A, 10% G, 10% T
  misc_feature (63)..(63): n = 70% G, 10% A, 10% C, 10% T
  misc_feature (64)..(64): n = 70% A, 10% G, 10% C, 10% T
  misc_feature (65)..(66): n = 70% C, 10% A, 10% G, 10% T
  misc_feature (67)..(67): n = 70% T, 10% A, 10% G, 10% C
  misc_feature (68)..(68): n = 70% A, 10% G, 10% C, 10% T
  misc_feature (69)..(69): n = 70% C, 10% A, 10% G, 10% T
  misc_feature (70)..(71): n = 70% A, 10% G, 10% C, 10% T
  misc_feature (72)..(72): n = 70% T, 10% A, 10% G, 10% C
  misc_feature (73)..(73): n = 70% G, 10% A, 10% C, 10% T
  misc_feature (74)..(74): n = 70% C, 10% A, 10% G, 10% T
  misc_feature (75)..(75): n = 70% T, 10% A, 10% G, 10% C
  misc_feature (76)..(76): n = 70% C, 10% A, 10% G, 10% T
  misc_feature (76)..(76): n = 70% C, 10% A, 10% G, 10% T
  misc_feature (77)..(78): n = 70% T, 10% A, 10% G, 10% C
  misc_feature (79)..(79): n = 70% C, 10% A, 10% G, 10% T
  misc_feature (80)..(80): n = 70% A, 10% G, 10% C, 10% T
  misc_feature (81)..(81): n = 70% C, 10% A, 10% G, 10% T

SEQ ID NO: 126 - protein, 15 aa, Artificial Sequence
  CDR-1
CAISGFNIKDTYIDW

SEQ ID NO: 127 - protein, 15 aa, Artificial Sequence
  CDR-1
  MOD_RES (1)..(1): Xaa = S/C
XAISGFSSSSSSIDW

SEQ ID NO: 128 - protein, 15 aa, Artificial Sequence
  CDR-1
  misc_feature (7)..(7): Xaa can be any naturally occurring amino acid
  MOD_RES (8)..(8): Xaa= F/L/I/V
  misc_feature (9)..(10): Xaa can be any naturally occurring amino acid
  misc_feature (12)..(12): Xaa can be any naturally occurring amino acid
SAISGFXXXXTXIDW

SEQ ID NO: 129 - protein, 15 aa, Artificial Sequence
  CDR-1
  misc_feature (7)..(7): Xaa can be any naturally occurring amino acid
  MOD_RES (8)..(8): Xaa= F/L/I/V
  misc_feature (9)..(10): Xaa can be any naturally occurring amino acid
  misc_feature (12)..(12): Xaa can be any naturally occurring amino acid
CAISGFXXXXTXIDW

SEQ ID NO: 130 - protein, 14 aa, Artificial Sequence
  CDR-2
ARIYPTNGYTRYAD

SEQ ID NO: 131 - protein, 14 aa, Artificial Sequence
  CDR-2
ARISPSSGSTSYAD

SEQ ID NO: 132 - protein, 14 aa, Artificial Sequence
  CDR-2
  misc_feature (4)..(4): Xaa can be any naturally occurring amino acid
  misc_feature (6)..(7): Xaa can be any naturally occurring amino acid
  misc_feature (9)..(9): Xaa can be any naturally occurring amino acid
  misc_feature (11)..(11): Xaa can be any naturally occurring amino acid
ARIXPXXGXTXYAD

SEQ ID NO: 133 - protein, 12 aa, Artificial Sequence
  CDR-2
CSRWGGDFYAMD

(19) United States
(12) Patent Application Publication
(10) Pub. No.: US 2024/0132580 A1
(43) Pub. Date: Apr. 25, 2024

(54) VEGFA-BINDING MOLECULES

(71) Applicant: DotBio Pte. Ltd., Singapore (SG)

(72) Inventor: Ignacio Asial, Singapore (SG)

(73) Assignee: DotBio Pte. Ltd., Singapore (SG)

(21) Appl. No.: 18/277,749

(22) PCT Filed: Feb. 18, 2022
(86) PCT No.: PCT/EP2022/054137

§ 371 Date: Aug. 16, 2023

(30) Foreign Application Priority Data
Feb. 19, 2021 (SG) 10202101681W

(51) Int. Cl. C07K 16/22; G01N 33/74

(52) U.S. Cl. CPC C07K 16/22; G01N 33/74; A61K 2039/505; C07K 2317/33

(57) ABSTRACT

VEGFA-binding molecules are disclosed. Also disclosed are nucleic acids and expression vectors encoding, compositions comprising, and methods using, the VEGFA-binding molecules.

This application claims priority from SG 10202101681W filed 19 Feb. 2022, the contents and elements of which are herein incorporated by reference for all purposes.

### TECHNICAL FIELD

The present disclosure relates to the fields of molecular biology, more specifically antigen-binding molecule technology. The present invention also relates to methods of medical treatment and prophylaxis.

### BACKGROUND

Anti-VEGF therapies are employed to treat diverse conditions, particularly in the oncology and ophthalmology fields (1-3). Developing humanized and stabilized antibody domains against VEGF is desirable due to their small size and modularity. Having a small size and high stability are desirable for ophthalmology applications, as this could enable topical delivery of the drug (4,5). Modularity, i.e. the ability of the domain antibody to fold autonomously and be fused to other domain antibodies or other proteins without compromising its integrity, is highly desirable for the development of multi-valent and multi-specific molecules. Indeed it simplifies the process of increasing valency and specificity by fusing the antibody domains in tandem, to monoclonal antibodies, or any other fusion protein (6-8).

### SUMMARY

In a first aspect the present disclosure provides an antigen-binding molecule, optionally isolated, which binds to VEGFA, wherein the antigen-binding molecule comprises a single domain antibody sequence incorporating the following CDRs:
CDR1 having the amino acid sequence of SEQ ID NO:45
CDR2 having the amino acid sequence of SEQ ID NO:46
CDR3 having the amino acid sequence of SEQ ID NO:48.

In some embodiments, the antigen-binding molecule comprises, or consists of, an amino acid sequence having at least 70% sequence identity to the amino acid sequence of SEQ ID NO:49.

In some embodiments, the antigen-binding molecule comprises a single domain antibody sequence incorporating the following FRs:
FR1 having the amino acid sequence of SEQ ID NO:40
FR2 having the amino acid sequence of SEQ ID NO:41
FR3 having the amino acid sequence of SEQ ID NO:47
FR4 having the amino acid sequence of SEQ ID NO:44.

In some embodiments, the antigen-binding molecule comprises a single domain antibody sequence incorporating the following CDRs:
CDR1 having the amino acid sequence of SEQ ID NO:50
CDR2 having the amino acid sequence of SEQ ID NO:51
CDR3 having the amino acid sequence of SEQ ID NO:52.

In some embodiments, the antigen-binding molecule comprises, or consists of, an amino acid sequence having at least 70% sequence identity to the amino acid sequence of SEQ ID NO:53.

In some embodiments, the antigen-binding molecule comprises:
(i) a single domain antibody sequence incorporating the following CDRs:
   CDR1 having the amino acid sequence of SEQ ID NO:17
   CDR2 having the amino acid sequence of SEQ ID NO:18
   CDR3 having the amino acid sequence of SEQ ID NO:19; or
(ii) a single domain antibody sequence incorporating the following CDRs:
   CDR1 having the amino acid sequence of SEQ ID NO:6
   CDR2 having the amino acid sequence of SEQ ID NO:7
   CDR3 having the amino acid sequence of SEQ ID NO:8; or
(iii) a single domain antibody sequence incorporating the following CDRs:
   CDR1 having the amino acid sequence of SEQ ID NO:13
   CDR2 having the amino acid sequence of SEQ ID NO:14
   CDR3 having the amino acid sequence of SEQ ID NO:15.

In some embodiments, the antigen-binding molecule comprises, or consists of, an amino acid sequence having at least 70% sequence identity to the amino acid sequence of SEQ ID NO:16, 5 or 12.

In some embodiments, the antigen-binding molecule comprises a single domain antibody sequence incorporating the following CDRs:
CDR1 having the amino acid sequence of SEQ ID NO:54
CDR2 having the amino acid sequence of SEQ ID NO:55
CDR3 having the amino acid sequence of SEQ ID N0:56.

In some embodiments, the antigen-binding molecule comprises, or consists of, an amino acid sequence having at least 70% sequence identity to the amino acid sequence of SEQ ID NO:57.

In some embodiments, the antigen-binding molecule comprises:
(i) a single domain antibody sequence incorporating the following CDRs:
   CDR1 having the amino acid sequence of SEQ ID NO:21
   CDR2 having the amino acid sequence of SEQ ID NO:22
   CDR3 having the amino acid sequence of SEQ ID NO:23; or
(ii) a single domain antibody sequence incorporating the following CDRs:
   CDR1 having the amino acid sequence of SEQ ID NO:25
   CDR2 having the amino acid sequence of SEQ ID NO:26
   CDR3 having the amino acid sequence of SEQ ID NO:27; or
(iii) a single domain antibody sequence incorporating the following CDRs:
   CDR1 having the amino acid sequence of SEQ ID NO:29
   CDR2 having the amino acid sequence of SEQ ID NO:30
   CDR3 having the amino acid sequence of SEQ ID NO:31; or
(iv) a single domain antibody sequence incorporating the following CDRs:
   CDR1 having the amino acid sequence of SEQ ID NO:33
   CDR2 having the amino acid sequence of SEQ ID NO:34
   CDR3 having the amino acid sequence of SEQ ID NO:35; or
(v) a single domain antibody sequence incorporating the following CDRs:
   CDR1 having the amino acid sequence of SEQ ID NO:37
   CDR2 having the amino acid sequence of SEQ ID NO:38
   CDR3 having the amino acid sequence of SEQ ID NO:39.

In some embodiments, the antigen-binding molecule comprises, or consists of, an amino acid sequence having at least 70% sequence identity to the amino acid sequence of SEQ ID NO:20, 24, 28, 32 or 36.

In some embodiments, the antigen-binding molecule comprises a single domain antibody sequence incorporating the following CDRs:
CDR1 having the amino acid sequence of SEQ ID NO:2
CDR2 having the amino acid sequence of SEQ ID NO:3
CDR3 having the amino acid sequence of SEQ ID NO:4.

In some embodiments, the antigen-binding molecule comprises, or consists of, an amino acid sequence having at least 70% sequence identity to the amino acid sequence of SEQ ID NO:1.

In some embodiments, the antigen-binding molecule comprises a single domain antibody sequence incorporating the following CDRs:
CDR1 having the amino acid sequence of SEQ ID NO:2
CDR2 having the amino acid sequence of SEQ ID NO:10
CDR3 having the amino acid sequence of SEQ ID NO:11.

In some embodiments, the antigen-binding molecule comprises, or consists of, an amino acid sequence having at least 70% sequence identity to the amino acid sequence of SEQ ID NO:9.

In some embodiments, the antigen-binding molecule comprises a single domain antibody sequence incorporating the following FRs:
FR1 having the amino acid sequence of SEQ ID NO:40
FR2 having the amino acid sequence of SEQ ID NO:41
FR3 having the amino acid sequence of SEQ ID NO:42
FR4 having the amino acid sequence of SEQ ID NO:44.

In some embodiments, the antigen-binding molecule comprises a single domain antibody sequence incorporating the following FRs:
FR1 having the amino acid sequence of SEQ ID NO:40
FR2 having the amino acid sequence of SEQ ID NO:41
FR3 having the amino acid sequence of SEQ ID NO:43
FR4 having the amino acid sequence of SEQ ID N0:44.

In some embodiments, the antigen-binding molecule inhibits interaction between VEGFA and VEGFR.

In some embodiments, the antigen-binding molecule is a multispecific antigen-binding molecule further comprising an antigen-binding domain specific for a target antigen other than VEGFA.

The present disclosure also provides a chimeric antigen receptor (CAR) comprising an antigen-binding molecule described herein.

The present disclosure also provides a nucleic acid, optionally isolated, encoding an antigen-binding molecule or a CAR described herein.

The present disclosure also provides an expression vector comprising a nucleic acid described herein.

The present disclosure also provides a cell comprising an antigen-binding molecule, CAR, nucleic acid or expression vector described herein.

The present disclosure also provides a method for producing an antigen-binding molecule which binds to VEGFA, comprising culturing a cell described herein under conditions suitable for expression of an antigen-binding molecule by the cell.

The present disclosure also provides a composition comprising an antigen-binding molecule, CAR, nucleic acid, expression vector or cell described herein, and a pharmaceutically acceptable carrier, diluent, excipient or adjuvant.

The present disclosure also provides an antigen-binding molecule, CAR, nucleic acid, expression vector, cell or composition described herein, for use in a method of medical treatment or prophylaxis.

The present disclosure also provides an antigen-binding molecule, CAR, nucleic acid, expression vector, cell or composition described herein, for use in a method of treatment or prevention of a disease in which VEGFA/VEGFR-mediated signalling is pathologically-implicated.

The present disclosure also provides the use of an antigen-binding molecule, CAR, nucleic acid, expression vector, cell or composition described herein, in the manufacture of a medicament for treating or preventing a disease in which VEGFA/VEGFR-mediated signalling is pathologically-implicated.

The present disclosure also provides a method of treating or preventing a disease in which VEGFA/VEGFR-mediated signalling is pathologically-implicated, comprising administering to a subject a therapeutically or prophylactically effective amount of an antigen-binding molecule, CAR, nucleic acid, expression vector, cell or composition described herein.

In some embodiments, the disease is selected from: a disease characterised by pathological angiogenesis, a cancer, a VEGFA-expressing cancer, a VEGFR-expressing cancer, an ocular disease, retinopathy, diabetic retinopathy, macular degeneration, age-related macular degeneration, wet age-related macular degeneration, retinal vein occlusion, myopic choroidal neovascularisation, retinopathy of prematurity, neovascular glaucoma, central serous retinopathy, ocular tumor, corneal neovascularisation, an inflammatory disease, an autoimmune disease, arthritis, rheumatoid arthritis, osteoarthritis, psoriasis, multiple sclerosis, sepsis, motor neuron disease and amyotrophic lateral sclerosis.

The present disclosure also provides an in vitro complex, optionally isolated, comprising an antigen-binding molecule according described herein bound to VEGFA.

The present disclosure also provides a method for detecting VEGFA in a sample, comprising contacting a sample containing, or suspected to contain, VEGFA with an antigen-binding molecule described herein, and detecting the formation of a complex of the antigen-binding molecule with VEGFA.

The present disclosure also provides the use of an antigen-binding molecule described herein in a method for detecting, localizing or imaging VEGFA, or cells comprising or expressing VEGFA.

The present disclosure also provides a method of selecting or stratifying a subject for treatment with a VEGFA-targeted agent, the method comprising contacting, in vitro, a sample from the subject with an antigen-binding molecule described herein, and detecting the formation of a complex of the antigen-binding molecule with VEGFA.

The present disclosure also provides the use of an antigen-binding molecule described herein as an in vitro or in vivo diagnostic or prognostic agent.

The present disclosure also provides the use of an antigen-binding molecule described herein in a method for detecting, localizing or imaging a disease/condition characterised by expression of VEGFA.

### DESCRIPTION

We hereby describe the generation of humanized, stabilized and autonomous VH domain antibodies targeting human and murine VEGFA with high affinity, which are able to block the VEGF-VEGFR interaction with high potency. These VEGF-binding molecules can be used as building blocks to generate multivalent and multi-specific molecules, as exemplified by a bivalent anti-VEGFA molecule built as a tandem of two VH domain antibodies.

VEGFA

Vascular endothelial growth factor A (VEGFA) is the protein identified by UniProt P15692. Alternative splicing of mRNA encoded by the human VEGFA gene yields four main VEGFA isoforms: VEGF206 (SEQ ID NO:60), VEGF189 (SEQ ID NO:61), VEGF165 (SEQ ID NO:62) and VEGF121 (SEQ ID NO:63). Following processing to remove an N-terminal 26 amino acid signal peptide (SEQ ID NO:68), VEGF206, VEGF189, VEGF165 and VEGF121 respectively comprise the amino acid sequences shown in SEQ ID NOs:64 to 67. VEGF165 appears to be the dominant VEGFA isoform.

VEGFA is a growth factor, and is described e.g. in Holme and Zachary Genome Biol. (2005) 6(2): 209, and Claesson-Welsh and Welsh, J Intern Med. (2013) 273(2):114-27, both of which are hereby incorporated by reference in their entirety.

Vascular endothelial growth factors (VEGFs) are a family of secreted polypeptides having a conserved receptor-binding cystine-knot structure. VEGFA monomers associate via interchain disulphide bonds to form homodimers. VEGFA acts through a family of cognate receptor kinases expressed by endothelial cells, to stimulate blood-vessel formation.

VEGFA has important roles in normal vascular development, and also in diseases involving abnormal growth of blood vessels (e.g. cancers). VEGFA stimulates the growth of vascular endothelial cells derived from arteries, veins, and the lymphatic system, and induces angiogenesis in a variety of in vivo models (i.e. the formation of thin-walled endothelium-lined structures), inducing rapid elevations in microvascular permeability.

In this specification ‘VEGFA’ refers to VEGFA from any species, and includes isoforms, fragments, variants or homologues from any species. In some embodiments VEGFA is VEGFA from a mammal (e.g. a therian, placental, epitherian, preptotheria, archontan, primate (rhesus, cynomolgous, non-human primate or human)). In some embodiments, the VEGFA is human or mouse VEGFA.

As used herein, isoforms, fragments, variants or homologues of a given reference protein may be characterised as having at least 70% sequence identity, preferably one of 80%, 85%, 90%, 91%, 92%, 93%, 94%, 95%, 96%, 97%, 98%, 99% or 100% amino acid sequence identity to the amino acid sequence of the reference protein. A ‘fragment’ generally refers to a fraction of the reference protein. A ‘variant’ generally refers to a protein having an amino acid sequence comprising one or more amino acid substitutions, insertions, deletions or other modifications relative to the amino acid sequence of the reference protein, but retaining a considerable degree of sequence identity (e.g. at least 60%) to the amino acid sequence of the reference protein. An ‘isoform’ generally refers to a variant of the reference protein expressed by the same species as the species of the reference protein. A ‘homologue’ generally refers to a variant of the reference protein produced by a different species as compared to the species of the reference protein. Homologues include orthologues.

Isoforms of VEGFA of course include VEGF206 (UniProt P15692-1), VEGF189 (UniProt P15692-2), VEGF165 (UniProt P15692-4) and VEGF121 (UniProt P15692-9). Isoforms of VEGFA also include VEGF183 (UniProt P15692-3), VEGF148 (UniProt P15692-5), VEGF145 (UniProt P15692-6), VEGF165B (UniProt P15692-8), VEGF111 (UniProt P15692-10), L-VEGF165 (UniProt P15692-11), L-VEGF121 (UniProt P15692-12), L-VEGF189 (UniProt P15692-13), L-VEGF206 (UniProt P15692-14), VEGFA isoform 15 (UniProt P15692-15), VEGFA isoform 16 (UniProt P15692-16), VEGFA isoform 17 (UniProt P15692-17), and VEGFA isoform 18 (UniProt P15692-18).

Isoforms, fragments, variants or homologues of VEGFA may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature VEGFA isoform from a given species, e.g. human.

In some embodiments, the VEGFA is human VEGFA. In some embodiments, the VEGFA is mouse VEGFA.

Isoforms, fragments, variants or homologues may optionally be functional isoforms, fragments, variants or homologues, e.g. having a functional property/activity of the reference VEGFA (e.g. human VEGF165), as determined by analysis by a suitable assay for the functional property/activity. For example, an isoform, fragment, variant or homologue of VEGFA may display association with a VEGF receptor (e.g. VEGFR1, VEGFR2 and/or VEGFR3).

In some embodiments, the VEGFA comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:60, 61, 62 or 63.

In some embodiments, the VEGFA, or fragment thereof, comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:64, 65, 66 or 67.

VEGFs exert their biological effects through binding the VEGF receptors (VEGFRs) VEGFR1 (AAH39007.1 GI: 24660372), VEGFR2 (P35968.2 GI: 9087218) and VEGFR3 (AAA85215.1 GI: 1150991). Each receptor has extracellular binding domains for VEGF, a transmembrane sequence and intracellular tyrosine kinase moieties. VEGF binding to the extracellular receptor domain dimerizes the receptors and results in phosphorylation of the intracellular tyrosine kinase moieties. VEGFA has been shown to exert its biological effects primarily through VEGFR1 and VEGFR2.

In this specification VEGFR1′, VEGFR2′ and VEGFR2′ refer respectively to VEGFR1/VEGFR2/VEGFR3 from any species, and include isoforms, fragments, variants or homologues from any species. In some embodiments, the VEGFR1/VEGFR2/VEGFR3 is from a mammal (e.g. a therian, placental, epitherian, preptotheria, archontan, primate (rhesus, cynomolgous, non-human primate or human)). In some embodiments, the VEGFR1/VEGFR2/VEGFR3 is the human or mouse VEGFR1/VEGFR2/VEGFR3.

Isoforms, fragments, variants or homologues of VEGFR1/VEGFR2/VEGFR3 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature isoform of the relevant molecule from a given species, e.g. human.

Herein, ‘VEGFA/VEGFR-mediated signalling’ refers to signalling initiated by binding of VEGFA to a VEGF receptor. ‘Signalling’ refers to signal transduction and other cellular processes governing cellular activity.

VEGFA/VEGFR-mediated signalling is described e.g. in Geindreau et al., Int J Mol Sci. (2021) 22(9): 4871, which is hereby incorporated by reference in its entirety. VEGFA/VEGFR-mediated signalling progresses intracellularly through the PI3K/AKT, MAPK/ERK and PLC-γ pathways, and also through SCR and FAK, to promote cell survival, proliferation, cytoskeletal rearrangement, and effect changes in vascular permeability, vasodilation and promote angiogenesis.

Antigen-Binding Molecules

The present disclosure provides antigen-binding molecules capable of binding to (i.e. which bind to) VEGFA. The present disclosure provides antigen-binding molecules which bind specifically to VEGFA. Antigen-binding molecules according to the present disclosure may be provided in purified or isolated form, i.e. from other naturally-occurring biological material.

As used herein, an ‘antigen-binding molecule’ refers to a molecule which is capable of binding to a target antigen. The term ‘antigen-binding molecule’ encompasses monoclonal antibodies, polyclonal antibodies, monospecific and multispecific antibodies (e.g., bispecific antibodies), and antibody fragments (e.g. Fv, scFv, Fab, scFab, F(ab′)₂, Fab₂, diabodies, triabodies, scFv-Fc, minibodies, single domain antibodies (VHH), etc.) and aptamers.

More specifically, antigen-binding molecules according to the present disclosure comprise antigen-binding polypeptide moieties, which may be referred to as ‘antigen-binding domains’. In preferred embodiments, antigen-binding molecules according to the present disclosure comprise, or consist of, a single domain antibody which binds specifically to VEGFA.

Single domain antibodies (sdAbs)—also referred to variously in the art as ‘single variable domain on a heavy chain antibodies’, ‘VHHs’, ‘nanobodies’ and ‘heavy chain only antibodies (HcAbs)’, and sometimes referred to herein as DotBodies'—are described e.g. in Henry and MacKenzie, Front Immunol. (2018) 9:41 and Bever et al., Anal Bioanal Chem. (2016) 408(22): 5985-6002, both of which are hereby incorporated by reference in their entirety.

Single-domain antibodies are formed of a single, monomeric antibody variable domain. The first single-domain antibodies were engineered from heavy-chain antibodies found in camelids, and cartilaginous fishes also have heavy-chain antibodies.

Single-domain antibodies according to the present disclosure generally comprise three complementarity-determining regions CDRs: CDR1, CDR2 and CDR3. The three CDRs together define the paratope of the molecule, which is the part through which it binds to its target antigen.

Single domain antibodies further comprise framework regions (FRs) either side of each CDR, which provide a scaffold for the CDRs. From N-terminus to C-terminus, single-domain antibodies comprise the following structure: N term-[FR1]-[CDR1]-[FR2]-[CDR2]-[FR3]-[CDR3]-[FR4]-C term.

There are several different conventions for defining antibody CDRs and FRs, such as those described in Kabat et al., Sequences of Proteins of Immunological Interest, 5th Ed. Public Health Service, National Institutes of Health, Bethesda, MD (1991), Chothia et al., J. Mol. Biol. 196:901-917 (1987), and VBASE2, as described in Retter et al., Nucl. Acids Res. (2005) 33 (suppl 1): D671-D674.

In some embodiments, the antigen-binding molecule comprises the CDRs of a VEGFA-binding single domain antibody described herein, or comprises CDRs which are derived from a VEGFA-binding single domain antibody described herein. In some embodiments, the antigen-binding molecule comprises the FRs of a VEGFA-binding single domain antibody described herein, or comprises FRs which are derived from a VEGFA-binding single domain antibody described herein. In some embodiments, the antigen-binding molecule comprises the CDRs and the FRs of a VEGFA-binding single domain antibody described herein, or comprises CDRs and FRs which are derived from a VEGFA-binding single domain antibody described herein. That is, in some embodiments the antigen-binding molecule comprises the amino acid sequence of a VEGFA-binding single domain antibody described herein, or comprises amino acid sequence which is derived from a VEGFA-binding single domain antibody described herein. In some embodiments, the CDRs and FRs of antigen-binding molecules referred to herein are defined according to the IMGT information system (international IMGT (ImMunoGeneTics) information system (described in LeFranc et al., Nucleic Acids Res. (2015) 43 (Database issue):D413-22), which uses the IMGT V-DOMAIN numbering rules as described in Lefranc et al., Dev. Comp. Immunol. (2003) 27:55-77), the Kabat system (described in Kabat et al., Sequences of Proteins of Immunological Interest, 5th Ed. Public Health Service, National Institutes of Health, Bethesda, MD (1991)) or the Chothia system (described in Chothia et al., J. Mol. Biol. 196:901-917 (1987)).

In some embodiments, the CDRs and FRs of antigen-binding molecules (e.g. the single domain antibodies) referred to herein are defined according to the Kabat system. In some embodiments, in the amino acid sequences of SEQ ID NOs:1, 5, 9, 12, 16, 20, 24, 28, 32, 36, 49, 53 and 57: FR1 is formed by the amino acid sequence at positions 1 to 31; CDR1 is formed by the amino acid sequence at positions 32 to 36; FR2 is formed by the amino acid sequence at positions 37 to 50; CDR2 is formed by the amino acid sequence at positions 51 to 67; FR3 is formed by the amino acid sequence at positions 68 to 99; CDR3 is formed by the amino acid sequence at positions 100 to 119; and FR4 is formed by the amino acid sequence at positions 120 to 130.

As used herein, an amino acid sequence/domain which is “derived from” a reference amino acid sequence/domain comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the reference sequence.

In some embodiments the antigen-binding molecule comprises the CDRs, FRs and/or the complete amino acid sequence of a VEGFA-binding single domain antibody selected from: 13A6, 16A2.1, 16A6.1, 20A2.1, 20A3.1, 21A1.1, 21A8.1, 21D9.1, 21E6.1 and 23D5.1.

In some embodiments the antigen-binding molecule comprises the CDRs, FRs and/or the complete amino acid sequence of a VEGFA-binding single domain antibody having an amino acid sequence according to one of SEQ ID NOs:1, 5, 9, 12, 16, 20, 24, 28, 32, 36, 49, 53 or 57. In some embodiments the antigen-binding molecule comprises the CDRs CDRs 1, 2 and 3) of a VEGFA-binding single domain antibody having an amino acid sequence according to one of SEQ ID NOs:1, 5, 9, 12, 16, 20, 24, 28, 32, 36, 49, 53 or 57. In some embodiments the antigen-binding molecule comprises the FRs FRs 1, 2, 3 and 4) of a VEGFA-binding single domain antibody having an amino acid sequence according to one of SEQ ID NOs:1, 5, 9, 12, 16, 20, 24, 28, 32, 36, 49, 53 or 57. In some embodiments the antigen-binding molecule comprises the CDRs CDRs 1, 2 and 3) and FRs FRs 1, 2, 3 and 4) of a VEGFA-binding single domain antibody having an amino acid sequence according to one of SEQ ID NOs:1, 5, 9, 12, 16, 20, 24, 28, 32, 36, 49, 53 or 57.

In some embodiments the antigen-binding molecule comprises, or consists of, a single domain antibody sequence according to one of (1) to (13) below:
(1) (ConAll) a single domain antibody sequence comprising the following CDRs:
   CDR1 having the amino acid sequence of SEQ ID NO:45
   CDR2 having the amino acid sequence of SEQ ID NO:46
   CDR3 having the amino acid sequence of SEQ ID NO:48,
   or a variant thereof in which one or two or three amino acids in one or more of CDR1, CDR2, or CDR3 are substituted with another amino acid.
(2) (Con16A2.1 derived) a single domain antibody sequence comprising the following CDRs:
   CDR1 having the amino acid sequence of SEQ ID NO:50
   CDR2 having the amino acid sequence of SEQ ID NO:51
   CDR3 having the amino acid sequence of SEQ ID NO:52,
   or a variant thereof in which one or two or three amino acids in one or more of CDR1, CDR2, or CDR3 are substituted with another amino acid.
(3) (Con21/23) a single domain antibody sequence comprising the following CDRs:
   CDR1 having the amino acid sequence of SEQ ID NO:54
   CDR2 having the amino acid sequence of SEQ ID NO:55
   CDR3 having the amino acid sequence of SEQ ID NO:56,
   or a variant thereof in which one or two or three amino acids in one or more of CDR1, CDR2, or CDR3 are substituted with another amino acid.
(4) (13A6) a single domain antibody sequence comprising the following CDRs:
   CDR1 having the amino acid sequence of SEQ ID NO:2
   CDR2 having the amino acid sequence of SEQ ID NO:3
   CDR3 having the amino acid sequence of SEQ ID NO:4,
   or a variant thereof in which one or two or three amino acids in one or more of CDR1, CDR2, or CDR3 are substituted with another amino acid.
(5) (16A2.1) a single domain antibody sequence comprising the following CDRs:
   CDR1 having the amino acid sequence of SEQ ID NO:6
   CDR2 having the amino acid sequence of SEQ ID NO:7
   CDR3 having the amino acid sequence of SEQ ID NO:8,
   or a variant thereof in which one or two or three amino acids in one or more of CDR1, CDR2, or CDR3 are substituted with another amino acid.
(6) (16A6.1) a single domain antibody sequence comprising the following CDRs:
   CDR1 having the amino acid sequence of SEQ ID NO:2
   CDR2 having the amino acid sequence of SEQ ID NO:10
   CDR3 having the amino acid sequence of SEQ ID NO:11,
   or a variant thereof in which one or two or three amino acids in one or more of CDR1, CDR2, or CDR3 are substituted with another amino acid.
(7) (20A2.1) a single domain antibody sequence comprising the following CDRs:
   CDR1 having the amino acid sequence of SEQ ID NO:13
   CDR2 having the amino acid sequence of SEQ ID NO:14
   CDR3 having the amino acid sequence of SEQ ID NO:15,
   or a variant thereof in which one or two or three amino acids in one or more of CDR1, CDR2, or CDR3 are substituted with another amino acid.
(8) (20A3.1) a single domain antibody sequence comprising the following CDRs:
   CDR1 having the amino acid sequence of SEQ ID NO:17
   CDR2 having the amino acid sequence of SEQ ID NO:18
   CDR3 having the amino acid sequence of SEQ ID NO:19,
   or a variant thereof in which one or two or three amino acids in one or more of CDR1, CDR2, or CDR3 are substituted with another amino acid.
(9) (21A1.1) a single domain antibody sequence comprising the following CDRs:
   CDR1 having the amino acid sequence of SEQ ID NO:21
   CDR2 having the amino acid sequence of SEQ ID NO:22
   CDR3 having the amino acid sequence of SEQ ID NO:23,
   or a variant thereof in which one or two or three amino acids in one or more of CDR1, CDR2, or CDR3 are substituted with another amino acid.
(10) (21A8.1) a single domain antibody sequence comprising the following CDRs:
   CDR1 having the amino acid sequence of SEQ ID NO:25
   CDR2 having the amino acid sequence of SEQ ID NO:26
   CDR3 having the amino acid sequence of SEQ ID NO:27,
   or a variant thereof in which one or two or three amino acids in one or more of CDR1, CDR2, or CDR3 are substituted with another amino acid.
(11) (21 D9.1) a single domain antibody sequence comprising the following CDRs:
   CDR1 having the amino acid sequence of SEQ ID NO:29
   CDR2 having the amino acid sequence of SEQ ID NO:30
   CDR3 having the amino acid sequence of SEQ ID NO:31,
   or a variant thereof in which one or two or three amino acids in one or more of CDR1, CDR2, or CDR3 are substituted with another amino acid.
(12) (21E6.1) a single domain antibody sequence comprising the following CDRs:
   CDR1 having the amino acid sequence of SEQ ID NO:33
   CDR2 having the amino acid sequence of SEQ ID NO:34
   CDR3 having the amino acid sequence of SEQ ID NO:35,
   or a variant thereof in which one or two or three amino acids in one or more of CDR1, CDR2, or CDR3 are substituted with another amino acid.
(13) (23D5.1) a single domain antibody sequence comprising the following CDRs:
   CDR1 having the amino acid sequence of SEQ ID NO:37
   CDR2 having the amino acid sequence of SEQ ID NO:38
   CDR3 having the amino acid sequence of SEQ ID NO:39,
   or a variant thereof in which one or two or three amino acids in one or more of CDR1, CDR2, or CDR3 are substituted with another amino acid.

In some embodiments the antigen-binding molecule comprises, or consists of, a single domain antibody sequence according to one of (14) to (16) below:
(14) (ConAll) a single domain antibody sequence comprising the following FRs:
   FR1 having the amino acid sequence of SEQ ID NO:40
   FR2 having the amino acid sequence of SEQ ID NO:41
   FR3 having the amino acid sequence of SEQ ID NO:47
   FR4 having the amino acid sequence of SEQ ID NO:44,
   or a variant thereof in which one or two or three amino acids in one or more of FR1, FR2, FR3, or FR4 are substituted with another amino acid.
(15) (13A6, 16A2.1, 20A2.1, 20A3.1, 21A1.1, 21A8.1, 21D9.1, 21E6.1) a single domain antibody sequence comprising the following FRs:
   FR1 having the amino acid sequence of SEQ ID NO:40
   FR2 having the amino acid sequence of SEQ ID NO:41
   FR3 having the amino acid sequence of SEQ ID NO:42
   FR4 having the amino acid sequence of SEQ ID NO:44,
   or a variant thereof in which one or two or three amino acids in one or more of FR1, FR2, FR3, or FR4 are substituted with another amino acid.
(16) (16A6.1) a single domain antibody sequence comprising the following FRs:
FR1 having the amino acid sequence of SEQ ID NO:40
FR2 having the amino acid sequence of SEQ ID NO:41
FR3 having the amino acid sequence of SEQ ID NO:43
FR4 having the amino acid sequence of SEQ ID NO:44,
or a variant thereof in which one or two or three amino acids in one or more of FR1, FR2, FR3, or FR4 are substituted with another amino acid.

In some embodiments, the antigen-binding molecule comprises, or consists of, a single domain antibody sequence comprising the CDRs according to one of (1) to (13) above, and the FRs according to one of (14) to (16) above.

In some embodiments, the antigen-binding molecule comprises, or consists of, a single domain antibody sequence according to one of (17) to (29) below:
(17) (ConAll) a single domain antibody sequence comprising the CDRs according to (1) and the FRs according to (14).
(18) (Con16A2.1 derived) a single domain antibody sequence comprising the CDRs according to (2) and the FRs according to (15).
(19) (Con21/23) a single domain antibody sequence comprising the CDRs according to (3) and the FRs according to (15).
(20) (13A6) a single domain antibody sequence comprising the CDRs according to (4) and the FRs according to (15).
(21) (16A2.1) a single domain antibody sequence comprising the CDRs according to (5) and the FRs according to (15).
(22) (16A6.1) a single domain antibody sequence comprising the CDRs according to (6) and the FRs according to (16).
(23) (20A2.1) a single domain antibody sequence comprising the CDRs according to (7) and the FRs according to (15).
(24) (20A3.1) a single domain antibody sequence comprising the CDRs according to (8) and the FRs according to (15).
(25) (21A1.1) a single domain antibody sequence comprising the CDRs according to (9) and the FRs according to (15).
(26) (21A8.1) a single domain antibody sequence comprising the CDRs according to (10) and the FRs according to (15).
(27) (21 D9.1) a single domain antibody sequence comprising the CDRs according to (11) and the FRs according to (15).
(28) (21E6.1) a single domain antibody sequence comprising the CDRs according to (12) and the FRs according to (15).
(29) (23D5.1) a single domain antibody sequence comprising the CDRs according to (13) and the FRs according to (15).

In some embodiments, the antigen-binding molecule comprises, or consists of, a single domain antibody sequence according to one of (30) to (42) below:
(30) (ConAll) a single domain antibody sequence comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100% sequence identity, to the amino acid sequence of SEQ ID NO:49.
(31) (Con16A2.1 derived) a single domain antibody sequence comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100% sequence identity, to the amino acid sequence of SEQ ID NO:53.
(32) (Con21/23) a single domain antibody sequence comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100% sequence identity, to the amino acid sequence of SEQ ID NO:57.
(33) (13A6) a single domain antibody sequence comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100% sequence identity, to the amino acid sequence of SEQ ID NO:1.
(34) (16A2.1) a single domain antibody sequence comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100% sequence identity, to the amino acid sequence of SEQ ID NO:5.
(35) (16A6.1) a single domain antibody sequence comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100% sequence identity, to the amino acid sequence of SEQ ID NO:9.
(36) (20A2.1) a single domain antibody sequence comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100% sequence identity, to the amino acid sequence of SEQ ID NO:12.
(37) (20A3.1) a single domain antibody sequence comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100% sequence identity, to the amino acid sequence of SEQ ID NO:16.
(38) (21A1.1) a single domain antibody sequence comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100% sequence identity, to the amino acid sequence of SEQ ID NO:20.
(39) (21A8.1) a single domain antibody sequence comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100% sequence identity, to the amino acid sequence of SEQ ID NO:24.
(40) (21D9.1) a single domain antibody sequence comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100% sequence identity, to the amino acid sequence of SEQ ID NO:28.
(41) (21E6.1) a single domain antibody sequence comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100% sequence identity, to the amino acid sequence of SEQ ID NO:32.
(42) (23D5.1) a single domain antibody sequence comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100% sequence identity, to the amino acid sequence of SEQ ID NO:36.

In embodiments in accordance with the present disclosure in which one or more amino acids are substituted with another amino acid, substitutions may be conservative substitutions, for example according to the following Table. In some embodiments, amino acids in the same block in the middle column are substituted. In some embodiments, amino acids in the same line in the rightmost column are substituted:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

In some embodiments, substitutions may be functionally conservative. That is, in some embodiments the substitution may not affect (or may not substantially affect) one or more functional properties (e.g. target binding) of the antigen-binding molecule comprising the substitution as compared to the equivalent unsubstituted molecule.

In some embodiments, the antigen-binding molecule of the present disclosure comprises one or more regions (e.g. CH1, CH2 and/or CH3) of an immunoglobulin heavy chain constant sequence. In some embodiments, the immunoglobulin heavy chain constant sequence is, or is derived from, the heavy chain constant sequence of an IgG (e.g. IgG1, IgG2, IgG3, IgG4), IgA (e.g. IgA1, IgA2), IgD, IgE or IgM, e.g. a human IgG (e.g. hIgG1, hIgG2, hIgG3, hIgG4), hIgA (e.g. hIgA1, hIgA2), hIgD, hIgE or hIgM. In some embodiments the immunoglobulin heavy chain constant sequence is, or is derived from, the heavy chain constant sequence of a human IgG1 allotype (e.g. G1m1, G1m2, G1m3 or G1m17).

In some embodiments the antigen-binding molecule comprises an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:69.

In some embodiments the antigen-binding molecule comprises a CH1 region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:70. In some embodiments the antigen-binding molecule comprises a hinge region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:71. In some embodiments the antigen-binding molecule comprises a CH2 region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:72. In some embodiments the antigen-binding molecule comprises a CH3 region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:73.

In some embodiments, the antigen-binding molecules of the present disclosure comprise an Fc region. An Fc region is composed of CH2 and CH3 regions from one polypeptide, and CH2 and CH3 regions from another polypeptide. The CH2 and CH3 regions from the two polypeptides together form the Fc region.

Fc regions provide for interaction with Fc receptors and other molecules of the immune system to bring about functional effects. IgG Fc-mediated effector functions are reviewed e.g. in Jefferis et al., Immunol Rev 1998 163:59-76 (hereby incorporated by reference in its entirety), and are brought about through Fc-mediated recruitment and activation of immune cells (e.g. macrophages, dendritic cells, neutrophils, basophils, eosinophils, platelets, mast cells, NK cells and T cells) through interaction between the Fc region and Fc receptors expressed by the immune cells, recruitment of complement pathway components through binding of the Fc region to complement protein C1q, and consequent activation of the complement cascade. Fc-mediated functions include Fc receptor binding, antibody-dependent cellular cytotoxicity (ADCC), antibody-dependent cell-mediated phagocytosis (ADCP), complement-dependent cytotoxicity (CDC), formation of the membrane attack complex (MAC), cell degranulation, cytokine and/or chemokine production, and antigen processing and presentation.

In some embodiments, an antigen-binding molecule according to the present disclosure comprises an Fc region capable of potentiating/directing one or more of ADCC, ADCP, CDC against, and/or potentiating formation of a MAC on or cell degranulation of, a cell comprising/expressing VEGFA (e.g. a cell expressing VEGFA, and/or a complex comprising VEGFA at the cell surface).

Modifications to antibody Fc regions that influence Fc-mediated functions are known in the art, such as those described e.g. in Wang et al., Protein Cell (2018) 9(1):63-73, which is hereby incorporated by reference in its entirety. Exemplary Fc region modifications known to influence antibody effector function are summarised in Table 1 of Wang et al., Protein Cell (2018) 9(1):63-73.

Multispecific antigen-binding molecules are also contemplated. By “multispecific” it is meant that the antigen-binding molecule displays specific binding to more than one target. In some embodiments, the antigen-binding molecule is a bispecific antigen-binding molecule. In some embodiments, the antigen-binding molecule comprises at least two different antigen-binding domains.

In some embodiments, the antigen-binding molecule binds to VEGFA and another target (e.g. an antigen other than VEGFA), and so is at least bispecific. The term “bispecific” means that the antigen-binding molecule is able to bind specifically to at least two distinct antigenic determinants.

It will be appreciated that an antigen-binding molecule according to the present disclosure (e.g. a multispecific antigen-binding molecule) may comprise antigen-binding molecules capable of binding to the targets for which the antigen-binding molecule is specific. For example, an antigen-binding molecule which binds to VEGFA and an antigen other than VEGFA may comprise: (i) an antigen-binding molecule which binds to VEGFA, and (ii) an antigen-binding molecule which binds to an antigen other than VEGFA. In some embodiments, a component antigen-binding molecule of a larger antigen-binding molecule (e.g.

a multispecific antigen-binding molecule) may be referred to e.g. as an “antigen-binding domain” or “antigen-binding region” of the larger antigen-binding molecule.

It will also be appreciated that an antigen-binding molecule according to the present disclosure (e.g. a multispecific antigen-binding molecule) may comprise antigen-binding polypeptides or antigen-binding polypeptide complexes capable of binding to the targets for which the antigen-binding molecule is specific.

In some embodiments, the antigen other than VEGFA in a multispecific antigen-binding molecule is an immune cell surface molecule. In some embodiments, the antigen is a cancer cell antigen. In some embodiments the antigen is a receptor molecule, e.g. a cell surface receptor. In some embodiments the antigen is a cell signalling molecule, e.g. a cytokine, chemokine, interferon, interleukin or lymphokine. In some embodiments the antigen is a growth factor or a hormone.

A cancer cell antigen is an antigen which is expressed or over-expressed by a cancer cell. A cancer cell antigen may be any peptide/polypeptide, glycoprotein, lipoprotein, glycan, glycolipid, lipid, or fragment thereof. A cancer cell antigen's expression may be associated with a cancer. A cancer cell antigen may be abnormally expressed by a cancer cell (e.g. the cancer cell antigen may be expressed with abnormal localisation), or may be expressed with an abnormal structure by a cancer cell. A cancer cell antigen may be capable of eliciting an immune response. In some embodiments, the antigen is expressed at the cell surface of the cancer cell (i.e. the cancer cell antigen is a cancer cell surface antigen). In some embodiments, the part of the antigen which is bound by the antigen-binding molecule described herein is displayed on the external surface of the cancer cell (i.e. is extracellular). The cancer cell antigen may be a cancer-associated antigen. In some embodiments the cancer cell antigen is an antigen whose expression is associated with the development, progression or severity of symptoms of a cancer. The cancer-associated antigen may be associated with the cause or pathology of the cancer, or may be expressed abnormally as a consequence of the cancer. In some embodiments, the cancer cell antigen is an antigen whose expression is upregulated (e.g. at the RNA and/or protein level) by cells of a cancer, e.g. as compared to the level of expression by comparable non-cancerous cells (e.g. non-cancerous cells derived from the same tissue/cell type). In some embodiments, the cancer-associated antigen may be preferentially expressed by cancerous cells, and not expressed by comparable non-cancerous cells (e.g. non-cancerous cells derived from the same tissue/cell type). In some embodiments, the cancer-associated antigen may be the product of a mutated oncogene or mutated tumor suppressor gene. In some embodiments, the cancer-associated antigen may be the product of an overexpressed cellular protein, a cancer antigen produced by an oncogenic virus, an oncofetal antigen, or a cell surface glycolipid or glycoprotein.

An immune cell surface molecule may be any peptide/polypeptide, glycoprotein, lipoprotein, glycan, glycolipid, lipid, or fragment thereof expressed at or on the cell surface of an immune cell. In some embodiments, the part of the immune cell surface molecule which is bound by the antigen-binding molecule of the present disclosure is on the external surface of the immune cell (i.e. is extracellular). The immune cell surface molecule may be expressed at the cell surface of any immune cell. In some embodiments, the immune cell may be a cell of hematopoietic origin, e.g. a neutrophil, eosinophil, basophil, dendritic cell, lymphocyte, or monocyte. The lymphocyte may be e.g. a T cell, B cell, natural killer (NK) cell, NKT cell or innate lymphoid cell (ILC), or a precursor thereof (e.g. a thymocyte or pre-B cell). In some embodiments the antigen is a CD3 polypeptide (e.g. CD3ε, CD3δ, CD3γ or CD3ζ).

In some embodiments, multispecific antigen-binding molecules described herein display at least monovalent binding with respect to VEGFA, and also display at least monovalent binding with respect to an antigen other than VEGFA. Binding valency refers to the number of binding sites in an antigen-binding molecule for a given antigenic determinant.

In some embodiments the antigen-binding molecule comprises a single domain antibody capable of binding to VEGFA (e.g. as described herein), and an antigen-binding region (e.g. a polypeptide (e.g. a single domain antibody), Fv, Fab or antibody) capable of binding to an antigen other than VEGFA.

In some embodiments, an antigen-binding molecule comprises an immune cell-engaging moiety. In some embodiments, the antigen-binding molecule is an immune cell engager. Immune cell engagers are reviewed e.g. in Goebeler and Bargou, Nat. Rev. Clin. Oncol. (2020) 17: 418-434 and Ellerman, Methods (2019) 154:102-117, both of which are hereby incorporated by reference in their entirety.

Immune cell engager molecules comprise an antigen-binding region for a target antigen of interest, and an antigen-binding region for recruiting/engaging an immune cell of interest. Immune cell engagers recruit/engage immune cells through an antigen-binding region specific for an immune cell surface molecule.

In some embodiments, the antigen-binding molecule comprises a CD3 polypeptide-binding moiety (e.g. an antigen-binding domain capable of binding to a CD3 polypeptide). The best studied immune cells engagers are bispecific T cell engagers (BiTEs), which comprise a target antigen binding domain, and a CD3 polypeptide (typically CD3ε)-binding domain, through which the BiTE recruits T cells. Binding of the BiTE to its target antigen and to the CD3 polypeptide expressed by the T cell results in activation of the T cell, and ultimately directs T cell effector activity against cells expressing the target antigen. Other kinds of immune cell engagers are well known in the art, and include natural killer cell engagers such as bispecific killer engagers (BiKEs), which recruit and activate NK cells.

In some embodiments, the immune cell engaged by the immune cell engager is a T cell or an NK cell. In some embodiments, the immune cell engager is a T cell-engager.

Particular Exemplary Embodiments of the Antigen-Binding Molecules

In some embodiments, the antigen-binding molecule of the present disclosure comprises, or consists of, an amino acid sequence having at least 70%, preferably one of 75%, 80%, 85%, 90%, 91%, 92%, 93%, 94%, 95%, 96%, 97%, 98%, 99% or 100% amino acid sequence identity to SEQ ID NO:49.

In some embodiments, the antigen-binding molecule of the present disclosure comprises, or consists of, an amino acid sequence having at least 70%, preferably one of 75%, 80%, 85%, 90%, 91%, 92%, 93%, 94%, 95%, 96%, 97%, 98%, 99% or 100% amino acid sequence identity to SEQ ID NO:53.

In some embodiments, the antigen-binding molecule of the present disclosure comprises, or consists of, an amino acid sequence having at least 70%, preferably one of 75%, 80%, 85%, 90%, 91%, 92%, 93%, 94%, 95%, 96%, 97%, 98%, 99% or 100% amino acid sequence identity to SEQ ID NO:57.

In some embodiments, the antigen-binding molecule of the present disclosure comprises, or consists of, an amino acid sequence having at least 70%, preferably one of 75%, 80%, 85%, 90%, 91%, 92%, 93%, 94%, 95%, 96%, 97%, 98%, 99% or 100% amino acid sequence identity to SEQ ID NO:1.

In some embodiments, the antigen-binding molecule of the present disclosure comprises, or consists of, an amino acid sequence having at least 70%, preferably one of 75%, 80%, 85%, 90%, 91%, 92%, 93%, 94%, 95%, 96%, 97%, 98%, 99% or 100% amino acid sequence identity to SEQ ID NO:5.

In some embodiments, the antigen-binding molecule of the present disclosure comprises, or consists of, an amino acid sequence having at least 70%, preferably one of 75%, 80%, 85%, 90%, 91%, 92%, 93%, 94%, 95%, 96%, 97%, 98%, 99% or 100% amino acid sequence identity to SEQ ID NO:9.

In some embodiments, the antigen-binding molecule of the present disclosure comprises, or consists of, an amino acid sequence having at least 70%, preferably one of 75%, 80%, 85%, 90%, 91%, 92%, 93%, 94%, 95%, 96%, 97%, 98%, 99% or 100% amino acid sequence identity to SEQ ID NO:12.

In some embodiments, the antigen-binding molecule of the present disclosure comprises, or consists of, an amino acid sequence having at least 70%, preferably one of 75%, 80%, 85%, 90%, 91%, 92%, 93%, 94%, 95%, 96%, 97%, 98%, 99% or 100% amino acid sequence identity to SEQ ID NO:16.

In some embodiments, the antigen-binding molecule of the present disclosure comprises, or consists of, an amino acid sequence having at least 70%, preferably one of 75%, 80%, 85%, 90%, 91%, 92%, 93%, 94%, 95%, 96%, 97%, 98%, 99% or 100% amino acid sequence identity to SEQ ID NO:20.

In some embodiments, the antigen-binding molecule of the present disclosure comprises, or consists of, an amino acid sequence having at least 70%, preferably one of 75%, 80%, 85%, 90%, 91%, 92%, 93%, 94%, 95%, 96%, 97%, 98%, 99% or 100% amino acid sequence identity to SEQ ID NO:24.

In some embodiments, the antigen-binding molecule of the present disclosure comprises, or consists of, an amino acid sequence having at least 70%, preferably one of 75%, 80%, 85%, 90%, 91%, 92%, 93%, 94%, 95%, 96%, 97%, 98%, 99% or 100% amino acid sequence identity to SEQ ID NO:28.

In some embodiments, the antigen-binding molecule of the present disclosure comprises, or consists of, an amino acid sequence having at least 70%, preferably one of 75%, 80%, 85%, 90%, 91%, 92%, 93%, 94%, 95%, 96%, 97%, 98%, 99% or 100% amino acid sequence identity to SEQ ID NO:32.

In some embodiments, the antigen-binding molecule of the present disclosure comprises, or consists of, an amino acid sequence having at least 70%, preferably one of 75%, 80%, 85%, 90%, 91%, 92%, 93%, 94%, 95%, 96%, 97%, 98%, 99% or 100% amino acid sequence identity to SEQ ID NO:36.

Linkers and Additional Sequences

Antigen-binding molecules may comprise additional amino acids/sequences of amino acids in addition to the amino acid sequence required for binding to the target antigen. In some embodiments, such additional amino acids/sequences of amino acids are provided at the N-terminus of a single domain antibody sequence according to the present disclosure. In some embodiments, such additional amino acids/sequences of amino acids are provided at the C-terminus of a single domain antibody sequence according to the present disclosure. In some embodiments, such additional amino acids/sequences of amino acids are provided at the N-terminus and C-terminus of a single domain antibody sequence according to the present disclosure.

In some embodiments, the antigen-binding molecule comprises one or more linker sequences between amino acid subsequences. For example, a linker sequence may be provided at one or both ends of the antigen-binding domain of an antigen-binding molecule according to the present disclosure.

Linker sequences are known to the skilled person, and are described, for example in Chen et al., Adv Drug Deliv Rev (2013) 65(10): 1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv Drug Deliv Rev (2013) 65(10): 1357-1369. Flexible linker sequences often comprise high proportions of glycine and/or serine residues.

In some embodiments, the linker sequence comprises at least one glycine residue and/or at least one serine residue. In some embodiments the linker sequence consists of glycine and serine residues. In some embodiments, the linker sequence comprises one or more (e.g. one of 1, 2, 3, 4, 5, 6 or 7) copies (e.g. in tandem) of a sequence motif consisting of glycine and serine residues, e.g. G45. In some embodiments, the linker sequence has a length of 1-2, 1-3, 1-4, 1-5, 1-10, 1-15, 1-20, 1-25, or 1-30 amino acids. In some embodiments, a linker sequence comprises, or consists of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:58.

The antigen-binding molecules and polypeptides of the present disclosure may additionally comprise further amino acids or sequences of amino acids. For example, the antigen-binding molecules and polypeptides may comprise amino acid sequence(s) to facilitate expression, folding, trafficking, processing, purification or detection of the antigen-binding molecule/polypeptide. For example, the antigen-binding molecule/polypeptide may comprise a sequence encoding a His, (e.g. 6×His), Myc, GST, MBP, FLAG, HA, E, or Biotin tag, optionally at the N- or C-terminus of the antigen-binding molecule/polypeptide. In some embodiments the antigen-binding molecule/polypeptide comprises a detectable moiety, e.g. a fluorescent, luminescent, immuno-detectable, radio, chemical, nucleic acid or enzymatic label.

The antigen-binding molecules and polypeptides of the present disclosure may additionally comprise a signal peptide (also known as a leader sequence or signal sequence). Signal peptides normally consist of a sequence of 5-30 hydrophobic amino acids, which form a single alpha helix. Secreted proteins and proteins expressed at the cell surface often comprise signal peptides.

Signal peptides may be present at the N-terminus of the antigen-binding molecule/polypeptide. The signal peptide may provide for efficient trafficking and secretion of the antigen-binding molecule/polypeptide. Signal peptides are typically removed by cleavage, and thus are not comprised in the mature antigen-binding molecule/polypeptide secreted from a cell expressing the antigen-binding molecule.

Signal peptides are known for many proteins, and are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as SignalP (Petersen et al., 2011 Nature Methods 8: 785-786) or Signal-BLAST (Frank and Sippl, 2008 Bioinformatics 24: 2172-2176).

Labels and Conjugates

In some embodiments the antigen-binding molecules of the present disclosure additionally comprise a detectable moiety.

In some embodiments the antigen-binding molecule comprises a detectable moiety, e.g. a fluorescent label, phosphorescent label, luminescent label, immuno-detectable label (e.g. an epitope tag), radiolabel, chemical, nucleic acid or enzymatic label. The antigen-binding molecule may be covalently or non-covalently labelled with the detectable moiety.

Fluorescent labels include e.g. fluorescein, rhodamine, allophycocyanin, eosine and NDB, green fluorescent protein (GFP), chelates of rare earths such as europium (Eu), terbium (Tb) and samarium (Sm), tetramethyl rhodamine, Texas Red, 4-methyl umbelliferone, 7-amino-4-methyl coumarin, Cy3, and Cy5. Radiolabels include radioisotopes such as Iodine¹²³, Iodine¹²⁵, Iodine¹²⁶, Iodine¹³¹, Iodine¹³³, Bromine⁷⁷, Technetium^{99m}, Indium¹¹¹, Indium^{113m}, Gallium⁶⁷, Gallium⁶⁸, Ruthenium⁹⁵, Ruthenium⁹⁷, Ruthenium¹⁰³, Ruthenium¹⁰⁵, Mercury²⁰⁷, Mercury^{99m}, Rhenium^{99m}, Rhenium¹⁰¹, Rhenium¹⁰⁵, Scandium⁴⁷, Tellurium^{121m}, Tellurium^{122m}, Tellurium^{125m}, Thulium¹⁶⁵, Thulium¹⁶⁷, Thulium¹⁶⁸, Copper⁶⁷, Fluorine¹⁸, Yttrium⁹⁰, Palladium¹⁰⁰, Bismuth²¹⁷ and Antimony²¹¹. Luminescent labels include as radioluminescent, chemiluminescent (e.g. acridinium ester, luminol, isoluminol) and bioluminescent labels. Immuno-detectable labels include haptens, peptides/polypeptides, antibodies, receptors and ligands such as biotin, avidin, streptavidin or digoxigenin. Nucleic acid labels include aptamers. Enzymatic labels include e.g. peroxidase, alkaline phosphatase, glucose oxidase, beta-galactosidase and luciferase.

In some embodiments the antigen-binding molecules of the present disclosure are conjugated to a chemical moiety. The chemical moiety may be a moiety for providing a therapeutic effect. Antibody-drug conjugates are reviewed e.g. in Parslow et al., Biomedicines. 2016 September; 4(3):14. In some embodiments, the chemical moiety may be a drug moiety (e.g. a cytotoxic agent), such that the antigen-binding molecule displays cytotoxicity to a cell comprising/expressing VEGFA (e.g. a cell expressing VEGFA and/or a complex comprising VEGFA at the cell surface). In some embodiments, the drug moiety may be a chemotherapeutic agent. In some embodiments, the drug moiety is selected from calicheamicin, DM1, DM4, monomethylauristatin E (MMAE), monomethylauristatin F (MMAF), SN-38, doxorubicin, duocarmycin, D6.5 and PBD.

Functional Properties of the Antigen-Binding Molecules

The antigen-binding molecules described herein may be characterised by reference to certain functional properties. In some embodiments, the antigen-binding molecule described herein may possess one or more of the following properties:
binds to VEGFA (e.g. human VEGFA and/or mouse VEGFA);
inhibits interaction between VEGFA and VEGFR (i.e. a receptor for VEGFA, e.g. VEGFR1);
inhibits signalling mediated by VEGFA/VEGFR;
retains binding to VEGFA following heat treatment;
reduces the number/proportion of cells expressing VEGFA;
increases cell killing of cells expressing VEGFA.

It will be appreciated that a given antigen-binding molecule may display more than one of the properties recited in the preceding paragraph. A given antigen-binding molecule may be evaluated for the properties recited in the preceding paragraph using suitable assays. The assays may be e.g. in vitro assays, which may be cell-free or cell-based assays. Alternatively, the assays may be e.g. in vivo assays, i.e. performed in non-human animals. Assays may employ species labelled with detectable entities in order to facilitate their detection.

Analysis of the results of such assays may comprise determining the concentration at which 50% of the maximal level of the relevant activity is attained. The concentration of antigen-binding molecule at which 50% of the maximal level of the relevant activity is attained may be referred to as the ‘half-maximal effective concentration’ of the antigen-binding molecule in relation to the relevant activity, which may also be referred to as the ‘EC₅₀’. By way of illustration, the EC₅₀ of a given antigen-binding molecule for binding to VEGFA may be the concentration at which 50% of the maximal level of binding to the relevant species is achieved.

Depending on the property, the EC₅₀ may also be referred to as the ‘half-maximal inhibitory concentration’ or ‘IC₅₀’, this being the concentration of antigen-binding molecule at which 50% of the maximal level of inhibition of a given property is observed. By way of illustration, the IC₅₀ of a given antigen-binding molecule for inhibiting interaction between VEGFA and VEGFR (e.g. VEGFR1) may be the concentration at which 50% of the maximal level of inhibition is achieved.

The antigen-binding molecules and antigen-binding domains described herein preferably display specific binding to VEGFA. As used herein, “specific binding” refers to binding which is selective for the antigen, and which can be discriminated from non-specific binding to non-target antigen. An antigen-binding molecule/domain that specifically binds to a target molecule preferably binds the target with greater affinity, and/or with greater duration than it binds to other, non-target molecules.

The ability of a given polypeptide to bind specifically to a given molecule can be determined by analysis according to methods known in the art, such as by ELISA, Surface Plasmon Resonance (SPR; see e.g. Hearty et al., Methods Mol Biol (2012) 907:411-442), Bio-Layer Interferometry (see e.g. Lad et al., (2015) J Biomol Screen 20(4): 498-507), flow cytometry, or by a radiolabelled antigen-binding assay (RIA) enzyme-linked immunosorbent assay. Through such analysis binding to a given molecule can be measured and quantified. In some embodiments, the binding may be the response detected in a given assay.

In some embodiments, the extent of binding of the antigen-binding molecule to a non-target molecule is less than about 10% of the binding of the antibody to the target molecule as measured, e.g. by ELISA, SPR, Bio-Layer Interferometry or by RIA. Alternatively, binding specificity may be reflected in terms of binding affinity where the antigen-binding molecule binds with a dissociation constant (KD) that is at least 0.1 order of magnitude (i.e. 0.1×10ⁿ, where n is an integer representing the order of magnitude) greater than the KD of the antigen-binding molecule towards a non-target molecule. This may optionally be one of at least 0.2, 0.3, 0.4, 0.5, 0.6, 0.7, 0.8, 0.9, 1.0, 1.5, or 2.0.

Binding to VEGFA may be determined by Bio-Layer Interferometry, e.g. as described in Example 10 of the present disclosure.

In some embodiments, an antigen-binding molecule according to the present disclosure binds to VEGFA. In some embodiments, an antigen-binding molecule according to the present disclosure binds to a polypeptide complex comprising VEGFA.

In some embodiments, the antigen-binding molecule described herein binds to VEGFA with sub-micromolar affinity, i.e. K_{D}<1×10⁻⁶ M. In some embodiments, the antigen-binding molecule described herein binds to VEGFA with an affinity in the nanomolar range, i.e. K_{D}=9.9×10⁻⁷ to 1×10⁻⁹ M. In some embodiments, the antigen-binding molecule described herein binds to VEGFA with sub-nanomolar affinity, i.e. K_{D}<1×10⁻⁹ M. In some embodiments, the antigen-binding molecule described herein binds to VEGFA with an affinity in the picomolar range, i.e. K_{D}=9.9×10⁻¹⁰ to 1×10⁻¹² M. In some embodiments, the antigen-binding molecule described herein binds to VEGFA with sub-picomolar affinity, i.e. K_{D}<1×10⁻¹² M.

In some embodiments, the antigen-binding molecule described herein binds to VEGFA (e.g. human VEGF165) with a K_{D} of 5 μM or less, preferably one of ≤5 μM, ≤2 μM, ≤1 μM, ≤500 nM, ≤100 nM, ≤75 nM, ≤50 nM, ≤40 nM, ≤30 nM, ≤20 nM, ≤15 nM, ≤12.5 nM, ≤10 nM, ≤9 nM, ≤8 nM, ≤7 nM, ≤6 nM, ≤5 nM, ≤4 nM≤3 nM, ≤2 nM, ≤1 nM, ≤500 pM, ≤400 pM, ≤300 pM, ≤200 pM, ≤100 pM, ≤75 pM, ≤50 pM, ≤45 pM, 540 pM, ≤35 pM, ≤30 pM, ≤25 pM, ≤20 pM, ≤15 pM or ≤10 pM. In some embodiments, the antigen-binding molecule binds to VEGFA (e.g. human VEGF165) with an affinity of K_{D}=≤1 nM, ≤500 pM, ≤400 pM, ≤300 pM, ≤200 pM, ≤100 pM, ≤75 pM, ≤50 pM, ≤45 pM, ≤40 pM, ≤35 pM, ≤30 pM, ≤25 pM, ≤20 pM, 515 pM or ≤10 pM.

In some embodiments, an antigen-binding molecule according to the present disclosure binds to VEGFA (e.g. human VEGF165) with a K_{D} (e.g. as determined by BLI, e.g. as described in Example 10 of the present disclosure) which is similar to the K_{D} determined for ranibizumab (i.e. the molecule formed by association of the polypeptides of SEQ ID NOs:74 and 75), in the same assay. binds to VEGFA (e.g. human VEGF165) with a K_{D} (e.g. as determined by BLI, e.g. as described in Example 10 of the present disclosure) which is 0.5 times and ≤2 times, e.g. one of 0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times, ≥0.95 times and ≤1.1 times the K_{D} determined for ranibizumab, in the same assay.

In some embodiments, an antigen-binding molecule according to the present disclosure binds to VEGFA (e.g. human VEGF165) with a K_{D} (e.g. as determined by BLI, e.g. as described in Example 10 of the present disclosure) which is lower than the K_{D} determined for ranibizumab, in the same assay. In some embodiments, the antigen-binding molecule binds to VEGFA (e.g. human VEGF165) with a K_{D} (e.g. as determined by BLI, e.g. as described in Example 10 of the present disclosure) which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times or ≤0.5 times the K_{D} determined for ranibizumab, in the same assay.

In some embodiments, an antigen-binding molecule according to the present disclosure binds to VEGFA (e.g. human VEGF165) with a K_{D} (e.g. as determined by BLI, e.g. as described in Example 10 of the present disclosure) which is similar to the K_{D} determined for bevacizumab (i.e. the molecule formed by association of the polypeptides of SEQ ID NOs:76 and 77), in the same assay. binds to VEGFA (e.g. human VEGF165) with a K_{D} (e.g. as determined by BLI, e.g. as described in Example 10 of the present disclosure) which is 0.5 times and ≤2 times, e.g. one of 0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times, ≥0.95 times and ≤1.1 times the K_{D} determined for bevacizumab, in the same assay.

In some embodiments, an antigen-binding molecule according to the present disclosure binds to VEGFA (e.g. human VEGF165) with a K_{D} (e.g. as determined by BLI, e.g. as described in Example 10 of the present disclosure) which is lower than the K_{D} determined for bevacizumab, in the same assay. In some embodiments, the antigen-binding molecule binds to VEGFA (e.g. human VEGF165) with a K_{D} (e.g. as determined by BLI, e.g. as described in Example 10 of the present disclosure) which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times or ≤0.5 times the K_{D} determined for bevacizumab, in the same assay.

The antigen-binding molecules of the present disclosure may bind to a particular region of interest of VEGFA. Antigen-binding molecules according to the present disclosure may bind to linear epitope of VEGFA, consisting of a contiguous sequence of amino acids (i.e. an amino acid primary sequence). In some embodiments, an antigen-binding molecules may bind to a conformational epitope of VEGFA, consisting of a discontinuous sequence of amino acids of the amino acid sequence.

The region of a given target molecule to which an antigen-binding molecule binds can be determined by the skilled person using various methods well known in the art, including X-ray co-crystallography analysis of antibody-antigen complexes, peptide scanning, mutagenesis mapping, hydrogen-deuterium exchange analysis by mass spectrometry, phage display, competition ELISA and proteolysis-based ‘protection’ methods. Such methods are described, for example, in Gershoni et al., BioDrugs, 2007, 21(3):145-156, which is hereby incorporated by reference in its entirety.

In some embodiments, an antigen-binding molecule according to the present disclosure binds to the same region of VEGFA, or an overlapping region of VEGFA, to the region of VEGFA which is bound by an antigen-binding molecule comprising the CDRs, FRs and/or the complete amino acid sequence of a VEGFA-binding single domain antibody selected from: 13A6, 16A2.1, 16A6.1, 20A2.1, 20A3.1, 21A1.1, 21A8.1, 21D9.1, 21E6.1 and 23D5.1.

In some embodiments, an antigen-binding molecule according to the present disclosure binds to the region of VEGFA through which VEGFA binds to a VEGFR (e.g. VEGFR1 and/or VEGFR2).

In some embodiments, the antigen-binding molecule binds to VEGFA in the region which is bound by VEGFR (e.g. VEGFR1). In some embodiments, the antigen-binding molecule inhibits interaction between VEGFR (e.g. VEGFR1) and VEGFA. In some embodiments, the antigen-binding molecule is a competitive inhibitor of binding of VEGFR (e.g. VEGFR1) to VEGFA. In some embodiments, the antigen-binding molecule blocks VEGFA from binding to a VEGFR (e.g. VEGFR1). In some embodiments, the antigen-binding molecule occupies the region of VEGFA to which a VEGFR (e.g. VEGFR1) binds, thereby inhibiting interaction between VEGFR (e.g. VEGFR1) and VEGFA. In some embodiments, the antigen-binding molecule displaces a VEGFR (e.g. VEGFR1) from a complex comprising VEGFA and a VEGFR (e.g. VEGFR1).

The ability of an antigen-binding molecule to inhibit interaction between two factors can be determined for example by analysis of interaction in the presence of, or following incubation of one or both of the interaction partners with, the antibody/fragment. An example of a suitable assay to determine whether a given antigen-binding molecule inhibits interaction between two interaction partners is a competition ELISA assay. An antigen-binding molecule which inhibits a given interaction (e.g. between VEGFA and VEGFR) is identified by the observation of a reduction/decrease in the level of interaction between the interaction partners in the presence of—or following incubation of one or both of the interaction partners with—the antigen-binding molecule, as compared to the level of interaction in the absence of the antigen-binding molecule (or in the presence of an appropriate control antigen-binding molecule). Suitable analysis can be performed in vitro, e.g. using recombinant interaction partners or using cells expressing the interaction partners. Cells expressing interaction partners may do so endogenously, or may do so from nucleic acid introduced into the cell. For the purposes of such assays, one or both of the interaction partners and/or the antigen-binding molecule may be labelled or used in conjunction with a detectable entity for the purposes of detecting and/or measuring the level of interaction.

In some embodiments, an antigen-binding molecule according to the present disclosure inhibits interaction between VEGFA and VEGFR1 (e.g. human VEGF121 and human VEGFR1) with an IC₅₀ (e.g. as determined by competition ELISA, e.g. a competition ELISA as described in Example 11 of the present disclosure) of 10 μM or less, preferably one of ≤5 μM, ≤2 μM, ≤1 μM, ≤500 nM, ≤100 nM, ≤75 nM, ≤50 nM, ≤40 nM, ≤30 nM, ≤20 nM, ≤15 nM 512.5 nM, ≤10 nM, ≤9 nM, ≤8 nM, ≤7 nM, ≤6 nM, ≤5 nM, ≤4 nM or ≤3 nM.

In some embodiments, an antigen-binding molecule according to the present disclosure inhibits interaction between VEGFA and VEGFR1 (e.g. human VEGF121 and human VEGFR1) with an IC₅₀ (e.g. as determined by competition ELISA, e.g. a competition ELISA as described in Example 11 of the present disclosure) which is similar to the IC₅₀ for inhibition of such interaction determined for ranibizumab (i.e. the molecule formed by association of the polypeptides of SEQ ID NOs:74 and 75), in the same assay. In some embodiments, the antigen-binding molecule inhibits interaction between VEGFA and VEGFR1 (e.g. human VEGF121 and human VEGFR1) with an IC₅₀ (e.g. as determined by competition ELISA, e.g. a competition ELISA as described in Example 11 of the present disclosure) which 0.5 times and ≤2 times, e.g. one of 0.55 times and ≤1.9 times, ≥0.6 times and ≤1.8 times, ≥0.65 times and ≤1.7 times, 0.7 times and ≤1.6 times, 0.75 times and ≤1.5 times, ≥0.8 times and ≤1.4 times, ≥0.85 times and ≤1.3 times, ≥0.9 times and ≤1.2 times, 0.95 times and ≤1.1 times the IC₅₀ value for inhibition of such interaction by ranibizumab, in the same assay.

In some embodiments, an antigen-binding molecule according to the present disclosure inhibits interaction between VEGFA and VEGFR1 (e.g. human VEGF121 and human VEGFR1) with an IC₅₀ (e.g. as determined by competition ELISA, e.g. a competition ELISA as described in Example 11 of the present disclosure) which is lower than the IC₅₀ for inhibition of such interaction determined for ranibizumab, in the same assay. In some embodiments, the antigen-binding molecule inhibits interaction between VEGFA and VEGFR1 (e.g. human VEGF121 and human VEGFR1) with an IC₅₀ (e.g. as determined by competition ELISA, e.g. a competition ELISA as described in Example 11 of the present disclosure) which is less than 1 times, e.g. one of 50.99 times, ≤0.95 times, ≤0.9 times, ≤0.85 times, ≤0.8 times, ≤0.75 times, ≤0.7 times, ≤0.65 times, ≤0.6 times, ≤0.55 times or ≤0.5 times the IC₅₀ value for inhibition of such interaction by ranibizumab, in the same assay.

In some embodiments, an antigen-binding molecule according to the present disclosure inhibits interaction between VEGFA and VEGFR1 (e.g. human VEGF121 and human VEGFR1) with an IC₅₀ (e.g. as determined by competition ELISA, e.g. a competition ELISA as described in Example 11 of the present disclosure) which is similar to the IC₅₀ for inhibition of such interaction determined for bevacizumab (i.e. the molecule formed by association of the polypeptides of SEQ ID NOs:76 and 77), in the same assay. In some embodiments, the antigen-binding molecule inhibits interaction between VEGFA and VEGFR1 (e.g. human VEGF121 and human VEGFR1) with an IC₅₀ (e.g. as determined by competition ELISA, e.g. a competition ELISA as described in Example 11 of the present disclosure) which 0.5 times and ≤2 times, e.g. one of 0.55 times and ≤1.9 times, ≥0.6 times and ≤1.8 times, ≥0.65 times and ≤1.7 times, 0.7 times and ≤1.6 times, 0.75 times and ≤1.5 times, ≥0.8 times and ≤1.4 times, ≥0.85 times and ≤1.3 times, ≥0.9 times and ≤1.2 times, 0.95 times and ≤1.1 times the IC₅₀ value for inhibition of such interaction by bevacizumab, in the same assay.

In some embodiments, an antigen-binding molecule according to the present disclosure inhibits interaction between VEGFA and VEGFR1 (e.g. human VEGF121 and human VEGFR1) with an IC₅₀ (e.g. as determined by competition ELISA, e.g. a competition ELISA as described in Example 11 of the present disclosure) which is lower than the IC₅₀ for inhibition of such interaction determined for bevacizumab, in the same assay. In some embodiments, the antigen-binding molecule inhibits interaction between VEGFA and VEGFR1 (e.g. human VEGF121 and human VEGFR1) with an IC₅₀ (e.g. as determined by competition ELISA, e.g. a competition ELISA as described in Example 11 of the present disclosure) which is less than 1 times, e.g. one of 50.99 times, ≤0.95 times, ≤0.9 times, ≤0.85 times, ≤0.8 times, ≤0.75 times, ≤0.7 times, ≤0.65 times, ≤0.6 times, ≤0.55 times or ≤0.5 times the IC₅₀ value for inhibition of such interaction by bevacizumab, in the same assay.

In some embodiments, the antigen-binding molecule inhibits VEGFA/VEGFR-mediated signalling (i.e. signalling mediated by binding of VEGFA to a VEGFR). VEGFA/VEGFR-mediated signalling can be analysed using VEGFR-expressing cells e.g. in an assay for detecting and/or quantifying VEGFA/VEGFR-mediated signalling.

Suitable assays for investigating VEGFA/VEGFR-mediated signalling include assays for detecting the phosphorylation/activity/expression of factors which are phosphorylated/activated/expressed as a consequence of VEGFA/VEGFR-mediated signalling. Such assays may comprise contacting VEGFR-expressing cells with an antigen-binding molecule according to the present disclosure in the presence of VEGFA. Assays for investigating VEGFA/VEGFR-mediated signalling may comprise analysing signalling through the PI3K/AKT, MAPK/ERK and/or PLC-γ pathway, and/or through SCR and/or FAK.

In some embodiments, the antigen-binding molecule of the present disclosure is capable of inhibiting VEGFA/VEGFR-mediated signalling (e.g. signalling mediated by binding of human VEGF121 to human VEGFR1) to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, 50.3 times, ≤0.25 times, ≤0.2 times, ≤0.15 times, ≤0.1 times, ≤0.05 times, or ≤0.01 times the level of such signalling in the absence of the antigen-binding molecule (or in the presence of an appropriate control antigen-binding molecule).

In some embodiments, an antigen-binding molecule according to the present disclosure inhibits VEGFA/VEGFR-mediated signalling (e.g. signalling mediated by binding of human VEGF121 to human VEGFR1) with an IC₅₀ which is similar to the IC₅₀ for inhibition of such interaction determined for ranibizumab (i.e. the molecule formed by association of the polypeptides of SEQ ID NOs:74 and 75), in the same assay. In some embodiments, the antigen-binding molecule inhibits VEGFA/VEGFR-mediated signalling (e.g. signalling mediated by binding of human VEGF121 to human VEGFR1) with an IC₅₀ which is 0.5 times and ≤2 times, e.g. one of 0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, 0.85 times and ≤1.3 times, ≥0.9 times and ≤1.2 times, ≥0.95 times and ≤1.1 times the IC₅₀ value for inhibition of such signalling by ranibizumab, in the same assay.

In some embodiments, an antigen-binding molecule according to the present disclosure inhibits VEGFA/VEGFR-mediated signalling (e.g. signalling mediated by binding of human VEGF121 to human VEGFR1) with an IC₅₀ which is lower than the IC₅₀ for inhibition of such interaction determined for ranibizumab, in the same assay. In some embodiments, the antigen-binding molecule inhibits VEGFA/VEGFR-mediated signalling (e.g. signalling mediated by binding of human VEGF121 to human VEGFR1) with an IC₅₀ which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times or ≤0.5 times the IC₅₀ value for inhibition of such signalling by ranibizumab, in the same assay.

In some embodiments, an antigen-binding molecule according to the present disclosure inhibits VEGFA/VEGFR-mediated signalling (e.g. signalling mediated by binding of human VEGF121 to human VEGFR1) with an IC₅₀ which is similar to the IC₅₀ for inhibition of such interaction determined for bevacizumab (i.e. the molecule formed by association of the polypeptides of SEQ ID NOs:76 and 77), in the same assay. In some embodiments, the antigen-binding molecule inhibits VEGFA/VEGFR-mediated signalling (e.g. signalling mediated by binding of human VEGF121 to human VEGFR1) with an IC₅₀ which is 0.5 times and ≤2 times, e.g. one of 0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, 0.85 times and ≤1.3 times, ≥0.9 times and ≤1.2 times, ≥0.95 times and ≤1.1 times the IC₅₀ value for inhibition of such signalling by bevacizumab, in the same assay.

In some embodiments, an antigen-binding molecule according to the present disclosure inhibits VEGFA/VEGFR-mediated signalling (e.g. signalling mediated by binding of human VEGF121 to human VEGFR1) with an IC₅₀ which is lower than the IC₅₀ for inhibition of such interaction determined for bevacizumab, in the same assay. In some embodiments, the antigen-binding molecule inhibits VEGFA/VEGFR-mediated signalling (e.g. signalling mediated by binding of human VEGF121 to human VEGFR1) with an IC₅₀ which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times or ≤0.5 times the IC₅₀ value for inhibition of such signalling by bevacizumab, in the same assay.

In some embodiments, an antigen-binding molecule according to the present disclosure binds to VEGFA (e.g. human VEGFA) with similar affinity before and after heat treatment. Heat treatment may comprise incubation for 1 hour in an appropriate buffer (e.g. buffer comprising 0.1% BSA and 0.01% Tween-20 in PBS), at room temperature, 60° C., 70° C. or 80° C. Heat treatment may be performed as described in Example 12 of the present disclosure.

In some embodiments, the antigen-binding molecule displays similar affinity for VEGFA before heat treatment, and following heat treatment for 1 hour at room temperature. In some embodiments, the antigen-binding molecule displays similar affinity for VEGFA before heat treatment, and following heat treatment for 1 hour at 60° C. In some embodiments, the antigen-binding molecule displays similar affinity for VEGFA before heat treatment, and following heat treatment for 1 hour at 70° C. In some embodiments, the antigen-binding molecule displays similar affinity for VEGFA before heat treatment, and following heat treatment for 1 hour at 80° C.

Herein, a binding affinity which is ‘similar’ to a reference binding affinity means a binding affinity which is within 50%, e.g. within one of 40%, 45%, 30%, 25%, 20% 19%, 18%, 17%, 16%, 15%, 14%, 13%, 12%, 11%, 10%, 9%, 8%, 7%, 6%, 5%, 4%, 3%, 2% or 1% of the reference binding affinity, as determined in the same assay.

The K_{D} for binding to VEGFA (e.g. human VEGFA) may be similar before and after heat treatment. Herein, a ‘similar’ K_{D} value to a reference value may be ≥0.5 times and ≤2 times, e.g. one of ≥0.7 times and ≤1.5 times, ≥0.75 times and ≤1.25 times, ≥0.8 times and ≤1.2 times, ≥0.85 times and ≤1.15 times, ≥0.9 times and ≤1.1 times, ≥0.91 times and ≤1.09 times, ≥0.92 times and ≤1.08 times, ≥0.93 times and ≤1.07 times, ≥0.94 times and ≤1.06 times, ≥0.95 times and ≤1.05 times, ≥0.96 times and ≤1.04 times, ≥0.97 times and ≤1.03 times, ≥0.98 times and ≤1.02 times, or ≥0.99 times and ≤1.01 times the reference value.

In some embodiments, an antigen-binding molecule according to the present disclosure may potentiate (i.e. upregulate, enhance) cell killing of cells comprising/expressing VEGFA.

In some embodiments, an antigen-binding molecule according to the present disclosure is capable of reducing the number/proportion of cells comprising/expressing VEGFA. In some embodiments, an antigen-binding molecule according to the present disclosure is capable of depleting/enhancing depletion of such cells.

In some embodiments, an antigen-binding molecule according to the present disclosure reduces the number/proportion of cells comprising/expressing VEGFA to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the number/proportion of such cells observed in the absence of the antigen-binding molecule, or in the presence of the same quantity of an appropriate control antigen-binding molecule, in a given assay.

Antigen-binding molecules according to the present disclosure may comprise one or more moieties for potentiating a reduction in the number/proportion of cells comprising/expressing VEGFA. For example, an antigen-binding molecule according to the present disclosure may e.g. comprise an Fc region and/or a drug moiety.

In some embodiments, an antigen-binding molecule according to the present disclosure comprises an Fc region capable of potentiating/directing one or more of ADCC, ADCP, CDC against, and/or potentiating formation of a MAC on or cell degranulation of, a cell comprising/expressing VEGFA.

In some embodiments, an antigen-binding molecule according to the present disclosure is capable of potentiating/directing ADCC against a cell comprising/expressing VEGFA.

In some embodiments, an antigen-binding molecule according to the present disclosure comprises a drug moiety. The antigen-binding molecule may be conjugated to the drug moiety. Antibody-drug conjugates are reviewed e.g. in Parslow et al., Biomedicines. 2016 September; 4(3):14 (incorporated by reference hereinabove). In some embodiments, the drug moiety is or comprises a cytotoxic agent, such that the antigen-binding molecule displays cytotoxicity to a cell comprising/expressing VEGFA. In some embodiments the drug moiety is or comprises a chemotherapeutic agent.

In some embodiments, an antigen-binding molecule according to the present disclosure comprises an immune cell-engaging moiety. In some embodiments, the antigen-binding molecule comprises a CD3 polypeptide-binding moiety (e.g. an antigen-binding domain capable of binding to a CD3 polypeptide).

In some embodiments, an antigen-binding molecule according to the present disclosure is capable of potentiating/directing T cell-mediated cytolytic activity against a cell comprising/expressing VEGFA.

In some embodiments, an antigen-binding molecule according to the present disclosure reduces the number/proportion of cells comprising/expressing VEGFA to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the number/proportion of such cells observed in the absence of the antigen-binding molecule, or in the presence of the same quantity of an appropriate control antigen-binding molecule, in a given assay.

In some embodiments, an antigen-binding molecule according to the present disclosure increases the level of killing of cells comprising/expressing VEGFA to greater than 1 times, e.g. ≥1.5 times, ≥2 times, ≥3 times, ≥4 times, ≥5 times, ≥6 times, ≥7 times, ≥8 times, ≥9 times, ≥10 times, ≥15 times, ≥20 times, ≥30 times, ≥40 times or ≥50 times the level of killing of such cells observed in the absence of the antigen-binding molecule, or in the presence of the same quantity of an appropriate control antigen-binding molecule, in a given assay.

Chimeric Antigen Receptors (CARs)

The present disclosure also provides Chimeric Antigen Receptors (CARs) comprising the antigen-binding polypeptides of the present disclosure.

CARs are recombinant receptors that provide both antigen-binding and T cell activating functions. CAR structure and engineering is reviewed, for example, in Dotti et al., Immunol Rev (2014) 257(1), hereby incorporated by reference in its entirety. CARs comprise an antigen-binding region linked to a cell membrane anchor region and a signalling region. An optional hinge region may provide separation between the antigen-binding region and cell membrane anchor region, and may act as a flexible linker.

The CARs of the present disclosure comprise an antigen-binding region which comprises or consists of the antigen-binding molecule of the present disclosure, or which comprises or consists of a single domain antibody sequence according to the present disclosure. That is, an antigen-binding molecule/single domain antibody sequence according to the present disclosure is comprised in, or constitutes, the antigen-binding region of the CAR.

The cell membrane anchor region is provided between the antigen-binding region and the signalling region of the CAR and provides for anchoring the CAR to the cell membrane of a cell expressing a CAR, with the antigen-binding region in the extracellular space, and signalling region inside the cell. In some embodiments, the CAR comprises a cell membrane anchor region comprising or consisting of an amino acid sequence which comprises, consists of, or is derived from, the transmembrane region amino acid sequence for one of CD3-ζ, CD4, CD8 or CD28. As used herein, a region which is ‘derived from’ a reference amino acid sequence comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the reference sequence.

The signalling region of a CAR allows for activation of the T cell. The CAR signalling regions may comprise the amino acid sequence of the intracellular domain of CD3-ζ, which provides immunoreceptor tyrosine-based activation motifs (ITAMs) for phosphorylation and activation of the CAR-expressing T cell. Signalling regions comprising sequences of other ITAM-containing proteins such as FcγRI have also been employed in CARs (Haynes et al., 2001 J Immunol 166(1):182-187). Signalling regions of CARs may also comprise co-stimulatory sequences derived from the signalling region of co-stimulatory molecules, to facilitate activation of CAR-expressing T cells upon binding to the target protein. Suitable co-stimulatory molecules include CD28, OX40, 4-1BB, ICOS and CD27. In some cases CARs are engineered to provide for co-stimulation of different intracellular signalling pathways. For example, signalling associated with CD28 co-stimulation preferentially activates the phosphatidylinositol 3-kinase (PI3K) pathway, whereas the 4-1 BB-mediated signalling is through TNF receptor associated factor (TRAF) adaptor proteins. Signalling regions of CARs therefore sometimes contain co-stimulatory sequences derived from signalling regions of more than one co-stimulatory molecule. In some embodiments, the CAR of the present disclosure comprises one or more co-stimulatory sequences comprising or consisting of an amino acid sequence which comprises, consists of, or is derived from, the amino acid sequence of the intracellular domain of one or more of CD28, OX40, 4-1 BB, ICOS and CD27.

An optional hinge region may provide separation between the antigen-binding domain and the transmembrane domain, and may act as a flexible linker. Hinge regions may be derived from IgG1. In some embodiments, the CAR of the present disclosure comprises a hinge region comprising or consisting of an amino acid sequence which comprises, consists of, or is derived from, the amino acid sequence of the hinge region of IgG1.

Also provided is a cell comprising a CAR according to the present disclosure. The CAR according to the present disclosure may be used to generate CAR-expressing immune cells, e.g. CAR-T or CAR-NK cells. Engineering of CARs into immune cells may be performed during culture, in vitro.

The antigen-binding region of the CAR of the present disclosure may be provided with any suitable format, e.g. scFv, scFab, etc.

Nucleic Acids and Vectors

The present disclosure provides nucleic acid encoding antigen-binding molecules and CARs according to the present disclosure. In some embodiments the nucleic acids comprise or consist of DNA and/or RNA.

The present disclosure also provides vectors comprising nucleic acid according to the preceding paragraph.

Nucleic acids and vectors according to the present disclosure may be provided in purified or isolated form, i.e. from other nucleic acid, or naturally-occurring biological material.

The nucleotide sequence of a nucleic acid according to the present disclosure may be contained in a vector, e.g. an expression vector. A “vector” as used herein is a nucleic acid molecule used as a vehicle to transfer exogenous nucleic acid into a cell. The vector may be a vector for expression of the nucleic acid in the cell. Such vectors may include a promoter sequence operably linked to the nucleotide sequence encoding the sequence to be expressed. A vector may also include a termination codon and expression enhancers. Any suitable vectors, promoters, enhancers and termination codons known in the art may be used to express a peptide or polypeptide from a vector according to the present disclosure.

The term “operably linked” may include the situation where a selected nucleic acid sequence and regulatory nucleic acid sequence (e.g. promoter and/or enhancer) are covalently linked in such a way as to place the expression of nucleic acid sequence under the influence or control of the regulatory sequence (thereby forming an expression cassette). Thus, a regulatory sequence is operably linked to the selected nucleic acid sequence if the regulatory sequence is capable of effecting transcription of the nucleic acid sequence. The resulting transcript may then be translated into a desired peptide(s)/polypeptide(s).

Suitable vectors include plasmids, binary vectors, DNA vectors, mRNA vectors, viral vectors (e.g. gammaretroviral vectors (e.g. murine Leukemia virus (MLV)-derived vectors), lentiviral vectors, adenovirus vectors, adeno-associated virus vectors, vaccinia virus vectors and herpesvirus vectors), transposon-based vectors, and artificial chromosomes (e.g. yeast artificial chromosomes).

In some embodiments, the vector may be a eukaryotic vector, e.g. a vector comprising the elements necessary for expression of protein from the vector in a eukaryotic cell. In some embodiments, the vector may be a mammalian expression vector, e.g. comprising a cytomegalovirus (CMV) or SV40 promoter to drive protein expression.

Cells Comprising/Expressing the Antigen-Binding Molecules/CARs

The present disclosure also provides a cell comprising or expressing antigen-binding molecules and CARs according to the present disclosure. Also provided is a cell comprising or expressing a nucleic acid or vector according to the present disclosure.

The cell may be a eukaryotic cell, e.g. a mammalian cell. The mammal may be a primate (rhesus, cynomolgous, non-human primate or human) or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate).

In some embodiments, the cell is, or is derived from, a cell type commonly used for the expression of polypeptides for use in therapy in humans. Exemplary cells are described e.g. in Kunert and Reinhart, Appl Microbiol Biotechnol. (2016) 100:3451-3461 (hereby incorporated by reference in its entirety), and include e.g. CHO, HEK 293, PER.C6, NS0 and BHK cells.

The present disclosure also provides a method for producing a cell comprising a nucleic acid or vector according to the present disclosure, comprising introducing a nucleic acid or vector according to the present disclosure into a cell. In some embodiments, introducing an isolated nucleic acid(s) or vector(s) according to the present disclosure into a cell comprises transformation, transfection, electroporation or transduction (e.g. retroviral transduction).

The present disclosure also provides a method for producing a cell expressing/comprising an antigen-binding molecule/CAR according to the present disclosure, comprising introducing a nucleic acid or vector according to the present disclosure into a cell. In some embodiments, the methods additionally comprise culturing the cell under conditions suitable for expression of the nucleic acid/vector by the cell. In some embodiments, the methods are performed in vitro.

The present disclosure also provides cells obtained or obtainable by the methods according to the present disclosure.

Producing the antigen-binding molecules and polypeptides

Antigen-binding molecules and polypeptides according to the present disclosure may be prepared according to methods for the production of polypeptides known to the skilled person. Polypeptides may be prepared by chemical synthesis, e.g. liquid or solid phase synthesis. For example, peptides/polypeptides can by synthesised using the methods described in, for example, Chandrudu et al., Molecules (2013), 18: 4373-4388, which is hereby incorporated by reference in its entirety.

Alternatively, antigen-binding molecules and polypeptides may be produced by recombinant expression. Molecular biology techniques suitable for recombinant production of polypeptides are well known in the art, such as those set out in Green and Sambrook, Molecular Cloning: A Laboratory Manual (4th Edition), Cold Spring Harbor Press, 2012, and in Nat Methods. (2008); 5(2): 135-146 both of which are hereby incorporated by reference in their entirety. Methods for the recombinant production of antigen-binding molecules are also described in Frenzel et al., Front Immunol. (2013); 4: 217 and Kunert and Reinhart, Appl Microbiol Biotechnol. (2016) 100: 3451-3461, both of which are hereby incorporated by reference in their entirety.

For recombinant production according to the present disclosure, any cell suitable for the expression of polypeptides may be used. The cell may be a prokaryote or eukaryote. In some embodiments the cell is a prokaryotic cell, such as a cell of archaea or bacteria. In some embodiments the bacteria may be Gram-negative bacteria such as bacteria of the family Enterobacteriaceae, for example Escherichia coli. In some embodiments, the cell is a eukaryotic cell such as a yeast cell, a plant cell, insect cell or a mammalian cell, e.g. a cell described hereinabove.

In some cases, the cell is not a prokaryotic cell because some prokaryotic cells do not allow for the same folding or post-translational modifications as eukaryotic cells. In addition, very high expression levels are possible in eukaryotes and proteins can be easier to purify from eukaryotes using appropriate tags. Specific plasmids may also be utilised which enhance secretion of the protein into the media.

In some embodiments polypeptides may be prepared by cell-free-protein synthesis (CFPS), e.g. according to a system described in Zemella et al. Chembiochem (2015) 16(17): 2420-2431, which is hereby incorporated by reference in its entirety.

Production may involve culture or fermentation of a eukaryotic cell modified to express the polypeptide(s) of interest. The culture or fermentation may be performed in a bioreactor provided with an appropriate supply of nutrients, air/oxygen and/or growth factors. Secreted proteins can be collected by partitioning culture media/fermentation broth from the cells, extracting the protein content, and separating individual proteins to isolate secreted polypeptide(s). Culture, fermentation and separation techniques are well known to those of skill in the art, and are described, for example, in Green and Sambrook, Molecular Cloning: A Laboratory Manual (4th Edition; incorporated by reference herein above).

Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

Following culturing the cells that express the antigen-binding molecule, the antigen-binding molecule may be isolated or purified (e.g. from cell culture supernatant). Any suitable method for isolating/purifying polypeptides of interest produced by expression from cells in culture may be employed.

In order to isolate the polypeptide, it may be necessary to separate the cells from nutrient medium. If the polypeptide is secreted from the cells, the cells may be separated by centrifugation from the culture media that contains the secreted polypeptide of interest. If the polypeptide of interest collects within the cell, protein isolation may comprise centrifugation to separate cells from cell culture medium, treatment of the cell pellet with a lysis buffer, and cell disruption e.g. by sonification, rapid freeze-thaw or osmotic lysis.

It may then be desirable to isolate the polypeptide of interest from the supernatant or culture medium, which may contain other protein and non-protein components. A common approach to separating protein components from a supernatant or culture medium is by precipitation. Proteins of different solubilities are precipitated at different concentrations of precipitating agent such as ammonium sulfate. For example, at low concentrations of precipitating agent, water soluble proteins are extracted. Thus, by adding different increasing concentrations of precipitating agent, proteins of different solubilities may be distinguished. Dialysis may be subsequently used to remove ammonium sulfate from the separated proteins.

Other methods for distinguishing different proteins are known in the art, for example ion exchange chromatography and size chromatography. These may be used as an alternative to precipitation or may be performed subsequently to precipitation.

Once the polypeptide of interest has been isolated from culture it may be desired or necessary to concentrate the polypeptide. A number of methods for concentrating proteins are known in the art, such as ultrafiltration or lyophilisation.

Compositions

The present disclosure also provides compositions comprising the antigen-binding molecules, CARs, nucleic acids, expression vectors and cells described herein.

The antigen-binding molecules, CARs, nucleic acids, expression vectors and cells described herein may be formulated as pharmaceutical compositions or medicaments for clinical use and may comprise a pharmaceutically-acceptable carrier, diluent, excipient or adjuvant.

The compositions of the present disclosure may comprise one or more pharmaceutically-acceptable carriers (e.g. liposomes, micelles, microspheres, nanoparticles), diluents/excipients (e.g. starch, cellulose, a cellulose derivative, a polyol, dextrose, maltodextrin, magnesium stearate), adjuvants, fillers, buffers, preservatives (e.g. vitamin A, vitamin E, vitamin C, retinyl palmitate, selenium, cysteine, methionine, citric acid, sodium citrate, methyl paraben, propyl paraben), anti-oxidants (e.g. vitamin A, vitamin E, vitamin C, retinyl palmitate, selenium), lubricants (e.g. magnesium stearate, talc, silica, stearic acid, vegetable stearin), binders (e.g. sucrose, lactose, starch, cellulose, gelatin, polyethylene glycol (PEG), polyvinylpyrrolidone (PVP), xylitol, sorbitol, mannitol), stabilisers, solubilisers, surfactants (e.g., wetting agents), masking agents or colouring agents (e.g. titanium oxide).

The term ‘pharmaceutically-acceptable’ as used herein pertains to compounds, ingredients, materials, compositions, dosage forms, etc., which are, within the scope of sound medical judgment, suitable for use in contact with the tissues of the subject in question (e.g. a human subject) without excessive toxicity, irritation, allergic response, or other problem or complication, commensurate with a reasonable benefit/risk ratio. Each carrier, diluent, excipient, adjuvant, filler, buffer, preservative, anti-oxidant, lubricant, binder, stabiliser, solubiliser, surfactant, masking agent, colouring agent, flavouring agent or sweetening agent of a composition according to the present disclosure must also be ‘acceptable’ in the sense of being compatible with the other ingredients of the formulation. Suitable carriers, diluents, excipients, adjuvants, fillers, buffers, preservatives, anti-oxidants, lubricants, binders, stabilisers, solubilisers, surfactants, masking agents, colouring agents, flavouring agents or sweetening agents can be found in standard pharmaceutical texts, for example, Remington's ‘The Science and Practice of Pharmacy’ (Ed. A. Adejare), 23rd Edition (2020), Academic Press.

The composition may be formulated for topical, parenteral, systemic, intracavitary, intravenous, intra-arterial, intramuscular, intrathecal, intraocular, intraconjunctival, intratumoral, subcutaneous, intradermal, intrathecal, oral ortransdermal routes of administration. In some embodiments, a pharmaceutical composition/medicament may be formulated for administration by injection or infusion, or administration by ingestion.

Suitable formulations may comprise the antigen-binding molecule in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

In some embodiments the composition is formulated for injection or infusion, e.g. into a blood vessel or tissue/organ of interest.

The present disclosure also provides methods for the production of pharmaceutically useful compositions, such methods of production may comprise one or more steps selected from: producing an antigen-binding molecule, CAR, nucleic acid, expression vector or cell described herein; isolating an antigen-binding molecule, CAR, nucleic acid, expression vector or cell described herein; and/or mixing an antigen-binding molecule, CAR, nucleic acid, expression vector or cell described herein with a pharmaceutically-acceptable carrier, adjuvant, excipient or diluent.

For example, a further aspect the present disclosure relates to a method of formulating or producing a medicament or pharmaceutical composition for use in the treatment of a disease/condition (e.g. a disease/condition described herein), the method comprising formulating a pharmaceutical composition or medicament by mixing an antigen-binding molecule, CAR, nucleic acid, expression vector or cell described herein with a pharmaceutically-acceptable carrier, adjuvant, excipient or diluent.

Therapeutic and Prophylactic Applications

The antigen-binding molecules, CARs, nucleic acids, expression vectors, cells and compositions described herein find use in therapeutic and prophylactic methods.

The present disclosure provides an antigen-binding molecule, CAR, nucleic acid, expression vector, cell or composition described herein for use in a method of medical treatment or prophylaxis. Also provided is the use of an antigen-binding molecule, CAR, nucleic acid, expression vector, cell or composition described herein in the manufacture of a medicament for treating or preventing a disease or condition. Also provided is a method of treating or preventing a disease or condition, comprising administering to a subject a therapeutically or prophylactically effective amount of an antigen-binding molecule, CAR, nucleic acid, expression vector, cell or composition described herein.

Therapeutic or prophylactic intervention in accordance with the present disclosure may be effective to reduce the development or progression of a disease/condition, alleviation of the symptoms of a disease/condition or reduction in the pathology of a disease/condition. The intervention may be effective to prevent progression of the disease/condition, e.g. to prevent worsening of, or to slow the rate of development of, the disease/condition. In some embodiments the methods may lead to an improvement in the disease/condition, e.g. a reduction in the symptoms of the disease/condition or reduction in some other correlate of the severity/activity of the disease/condition. In some embodiments the methods may prevent development of the disease/condition a later stage (e.g. a more severe stage, or a chronic stage).

The terms ‘develop’, ‘developing’, and ‘development’, e.g. of a disorder, as used herein refer both to the onset of a disease as well as the progression, exacerbation or worsening of a disease state/correlate thereof.

It will be appreciated that the articles of the present disclosure may be used for the treatment/prevention of any disease/condition that would derive therapeutic or prophylactic benefit from a reduction in the level of VEGFA, VEGFA/VEGFR-mediated signalling, a reduction in the number of cells comprising/expressing VEGFA, and/or a reduction in the activity of cells expressing VEGFR. The disease/condition may e.g. be a disease/condition in which VEGFA, VEGFA/VEGFR-mediated signalling and/or cells comprising/expressing VEGFA/VEGFR are pathologically implicated, e.g. a disease/condition for which an elevated level of VEGFA/VEGFR-mediated signalling and/or an increased number of cells comprising/expressing VEGFA/VEGFR are positively associated with the onset, development or progression of the disease/condition, and/or severity of one or more symptoms of the disease/condition, or for which an elevated level of VEGFA/VEGFR-mediated signalling and/or an increased number of cells comprising/expressing VEGFA/VEGFR, is a risk factor for the onset, development or progression of the disease/condition.

Therapy and prophylaxis in accordance with the aspects and embodiments disclosed herein are concerned primarily with diseases/conditions characterised by VEGFA/VEGFR-mediated signalling.

In some embodiments, the disease/condition to be treated/prevented in accordance with the present disclosure is a disease/condition characterised by an increase in the level of expression of VEGFA, e.g. as compared to the level of expression of VEGFA in the absence of the disease/condition. In some embodiments, the disease/condition to be treated/prevented in accordance with the present disclosure is a disease/condition characterised by an increase in the number/proportion/activity of cells expressing VEGFR, e.g. as compared to the number/proportion/activity of cells expressing VEGFR in the absence of the disease/condition.

VEGFA/VEGFR-mediated signalling and its role in disease is reviewed e.g. in Karaman Development (2018) 145(14):dev151019, Ferrara and Adamis, Nat Rev Drug Discov. (2016) 15(6):385-403, and Claesson-Welsh and Welsh, J Intern Med. (2013) 273(2):114-27, all of which are hereby incorporated by reference in their entirety.

VEGFA/VEGFR-mediated signalling is implicated in pathogenesis of several diseases. VEGFA promotes angiogenesis, disruption of the blood-retinal barrier, inflammation and vision loss in individuals with ocular diseases such as diabetic retinopathy and wet age-related macular degeneration.

VEGFs and VEGF receptors are also expressed in non-endothelial cells, including some tumor cells. VEGFA secreted by tumor cells stimulates the proliferation and survival of endothelial cells, leading to the formation of new blood vessels, promoting tumor growth. The development and use of neutralizing antibodies to VEGFA produced the first direct evidence that tumor growth depends on angiogenesis and confirmed the importance of VEGFA in this process.

In some embodiments, the disease/condition to be treated in accordance with the present invention is selected from: a disease characterised by pathological (i.e. excessive) angiogenesis, a cancer, a VEGFA-expressing cancer (i.e. a cancer comprising cells expressing VEGFA; e.g. a cancer comprising cells having an elevated level of expression of VEGFA as compared to the level of expression by equivalent non-cancerous cells), a VEGFR-expressing cancer (i.e. a cancer comprising cells expressing VEGFR; e.g. a cancer comprising cells having an elevated level of expression of VEGFR as compared to the level of expression by equivalent non-cancerous cells), an ocular disease, retinopathy, diabetic retinopathy, macular degeneration, age-related macular degeneration, wet (i.e. neovascular) age-related macular degeneration, retinal vein occlusion, myopic choroidal neovascularisation, retinopathy of prematurity, neovascular glaucoma, central serous retinopathy, ocular tumor, corneal neovascularisation, an inflammatory disease, an autoimmune disease, arthritis, rheumatoid arthritis, osteoarthritis, psoriasis, multiple sclerosis, sepsis, motor neuron disease and amyotrophic lateral sclerosis.

It will be appreciated that certain of the diseases recited in the preceding paragraph are interrelated. For example, diseases characterised by pathological angiogenesis include cancers and ocular diseases. As used herein, ‘pathological angiogenesis’ refers to angiogenesis (i.e. the growth of new blood vessels from an existing vascular plexus), wherein the angiogenesis contributes to the development and/or progression of a disease.

In some embodiments, the disease/condition to be treated/prevented in accordance with the present disclosure is a cancer. The cancer may be any unwanted cell proliferation (or any disease manifesting itself by unwanted cell proliferation), neoplasm or tumor. The cancer may be benign or malignant and may be primary or secondary (metastatic). A neoplasm or tumor may be any abnormal growth or proliferation of cells and may be located in any tissue. The cancer may be of tissues/cells derived from e.g. the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, white blood cells.

Tumors to be treated may be nervous or non-nervous system tumors. Nervous system tumors may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumors may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T-cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, hematologic cancer and sarcoma.

The treatment/prevention may be aimed at one or more of: delaying/preventing the onset/progression of symptoms of the cancer, reducing the severity of symptoms of the cancer, reducing the survival/growth/invasion/metastasis of cells of the cancer, reducing the number of cells of the cancer and/or increasing survival of the subject.

In some embodiments, the cancer to be treated/prevented comprises cells expressing VEGFA. In some embodiments, the cancer to be treated/prevented comprises cells expressing VEGFR. In some embodiments, the cancer to be treated/prevented is a cancer which is positive for VEGFA. In some embodiments, the cancer to be treated/prevented is a cancer which is positive for VEGFR. In some embodiments, the cancer over-expresses VEGFA. In some embodiments, the cancer over-expresses VEGFR. Overexpression of VEGFA and/or VEGFR can be determined by detection of a level of expression of the relevant factor which is greater than the level of expression by equivalent non-cancerous cells/non-tumor tissue.

VEGFA and/or VEGFR expression may be determined by any suitable means. Expression may be gene expression or protein expression. Gene expression can be determined e.g. by detection of mRNA encoding VEGFA and/or VEGFR, for example by quantitative real-time PCR (qRT-PCR). Protein expression can be determined e.g. by detection of VEGFA and/or VEGFR, for example by antibody-based methods, for example by western blot, immunohistochemistry, immunocytochemistry, flow cytometry, or ELISA.

In some embodiments, a patient may be selected for treatment described herein based on the detection of a cancer expressing VEGFA and/or VEGFR, or overexpressing VEGFA and/or VEGFR, e.g. in a sample obtained from the subject.

VEGFA/VEGFR antagonists have been investigated as agents for the treatment/prevention of a wide variety of cancers, as described e.g. in Kieran et al., Cold Spring Harb Perspect Med. 2012 December; 2(12): a006593 (hereby incorporated by reference in its entirety; see e.g. Table 2). In some embodiments, the cancer to be treated/prevented in accordance with the present disclosure is selected from: a solid tumor, a hematologic malignancy, a myeloid hematologic malignancy, acute myeloid leukemia, multiple myeloma, breast cancer, renal cancer, renal cell carcinoma, lung cancer, non-small cell lung cancer, thyroid cancer, medullary thyroid cancer, brain/spinal cord cancer, glioblastoma, glioma, high-grade glioma, head and neck cancer, skin cancer, melanoma, squamous cell cancer, liver cancer, hepatocellular carcinoma, pancreatic cancer, gastric cancer, bowel cancer, colon cancer, rectal cancer, colorectal cancer, bile duct cancer, cholangiocarcinoma, bone cancer, sarcoma, ovarian cancer, cervical cancer, peritoneal cancer, prostate cancer, urothelial cancer, neuroendocrine cancer. In some embodiments, the cancer to be treated/prevented is a primary cancer. In some embodiments, the cancer the cancer to be treated/prevented is a secondary cancer (i.e. a metastasis).

VEGFA/VEGFR-targeted intervention has also been investigated for the treatment/prevention of ocular diseases, as described e.g. in Cornel et al., Rom J Ophthalmol. (2015) 59(4): 235-242, which is hereby incorporated by reference in its entirety. In some embodiments, the disease/condition to be treated/prevented in accordance with the present disclosure is selected from: an ocular disease, retinopathy, diabetic retinopathy, macular degeneration, age-related macular degeneration, wet (i.e. neovascular) age-related macular degeneration, retinal vein occlusion, myopic choroidal neovascularisation, retinopathy of prematurity, neovascular glaucoma, central serous retinopathy, ocular tumor and corneal neovascularisation.

VEGFA/VEGFR-mediated signalling has also been implicated in the pathology of inflammatory and autoimmune conditions, as described e.g. in Le and Kwon, Int J Mol Sci. (2021) 22(10):5387, Marina et al., Clujul Med. (2015) 88(3): 247-252, Ferrara, Endocr Rev. (2004) 25(4):581-611 and Azimi et al., Neurol Sci. (2020) 41(6):1459-1465, all of which are hereby incorporated by reference in their entirety. In some embodiments, the disease/condition to be treated/prevented in accordance with the present disclosure is selected from: an inflammatory disease, an autoimmune disease, arthritis, rheumatoid arthritis, osteoarthritis, psoriasis, multiple sclerosis and sepsis.

VEGFA/VEGFR-mediated signalling has also been implicated in the pathology of motor neuron disease such as amyotrophic lateral sclerosis, as described e.g. in Lambrechts et al., Nat Genet. (2003) 34(4):383-94. In some embodiments, the disease/condition to be treated/prevented in accordance with the present disclosure is motor neuron disease or amyotrophic lateral sclerosis.

In accordance with various aspects of the present disclosure, methods are provided which are for, or which comprise (e.g. in the context of therapeutic/prophylactic intervention as described herein), inhibiting interaction between VEGFA and VEGFR (i.e. a receptor for VEGFA, e.g. VEGFR1) and/or inhibiting VEGFA/VEGFR-mediated signalling.

Also provided are agents according to the present disclosure for use in such methods, and the use of agents according to the present disclosure in manufacture of compositions (e.g. medicaments) for use in such methods. In some embodiments, therapeutic/prophylactic intervention in accordance with the present disclosure may be described as being ‘associated with’ one or more of the effects described in the preceding paragraph. The skilled person is readily able to evaluate such properties using techniques that are routinely practiced in the art.

Administration of the articles of the present disclosure is preferably in a “therapeutically effective” or “prophylactically effective” amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's ‘The Science and Practice of Pharmacy’ (ed. A. Adejare), 23rd Edition (2020), Academic Press.

Administration may be alone or in combination with other treatments, either simultaneously or sequentially dependent upon the condition to be treated. The antigen-binding molecule or composition described herein and a therapeutic agent may be administered simultaneously or sequentially.

Simultaneous administration refers to administration of the antigen-binding molecule, CAR, nucleic acid, expression vector, cell or composition of the present disclosure and other therapeutic agent together, for example as a pharmaceutical composition containing both agents (i.e. in the case of a combined preparation), or immediately after one another, and optionally via the same route of administration, e.g. to the same artery, vein or other blood vessel. Sequential administration refers to administration of one of the agents, followed after a given time interval by separate administration of the other agent. It is not required that the two agents are administered by the same route, although this is the case in some embodiments. The time interval may be any time interval.

Multiple doses of the antigen-binding molecule, CAR, nucleic acid, expression vector, cell or composition may be provided. One or more, or each, of the doses may be accompanied by simultaneous or sequential administration of another therapeutic agent. Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

Methods of Detection

The present disclosure also provides the articles of the present disclosure for use in methods for detecting VEGFA, or methods for detecting cells comprising/expressing VEGFA.

The antigen-binding molecules described herein may be used in methods that involve detecting binding of the antigen-binding molecule to VEGFA. Such methods may involve detection of the bound complex of the antigen-binding molecule and VEGFA.

As such, a method is provided, comprising contacting a sample containing, or suspected to contain, VEGFA, and detecting the formation of a complex of the antigen-binding molecule and VEGFA. Also provided is a method comprising contacting a sample containing, or suspected to contain, a cell comprising/expressing VEGFA, and detecting the formation of a complex of the antigen-binding molecule and a cell comprising/expressing VEGFA.

Suitable method formats are well known in the art, including immunoassays such as sandwich assays, e.g. ELISA. The methods may involve labelling the antigen-binding molecule, or target(s), or both, with a detectable moiety, e.g. a fluorescent label, phosphorescent label, luminescent label, immuno-detectable label, radiolabel, chemical, nucleic acid or enzymatic label as described herein. Detection techniques are well known to those of skill in the art and can be selected to correspond with the labelling agent.

Methods comprising detecting VEGFA or cells comprising/expressing VEGFA include methods for diagnosing/prognosing diseases/conditions in which VEGFA expression/activity is pathologically-implicated.

Methods of this kind may be performed in vitro on a patient sample, or following processing of a patient sample. Once the sample is collected, the patient is not required to be present for the in vitro method to be performed, and therefore the method may be one which is not practised on the human or animal body. In some embodiments the method is performed in vivo.

Such methods may involve detecting or quantifying one or more of: VEGFA, cells comprising/expressing VEGFA, e.g. in a patient sample. Where the method comprises quantifying the relevant factor, the method may further comprise comparing the determined amount against a standard or reference value as part of the diagnostic or prognostic evaluation. Other diagnostic/prognostic tests may be used in conjunction with those described herein to enhance the accuracy of the diagnosis or prognosis or to confirm a result obtained by using the tests described herein.

Detection in a sample may be used for the purpose of diagnosis of a disease/condition (e.g. a cancer), predisposition to a disease/condition, or for providing a prognosis (prognosticating) for a disease/condition, e.g. a disease/condition described herein. The diagnosis or prognosis may relate to an existing (previously diagnosed) disease/condition.

A sample may be taken from any tissue or bodily fluid. The sample may comprise or may be derived from: a quantity of blood; a quantity of serum derived from the individual's blood which may comprise the fluid portion of the blood obtained after removal of the fibrin clot and blood cells; a tissue sample or biopsy; pleural fluid; cerebrospinal fluid (CSF); or cells isolated from said individual. In some embodiments, the sample may be obtained or derived from a tissue or tissues which are affected by the disease/condition (e.g. tissue or tissues in which symptoms of the disease manifest, or which are involved in the pathogenesis of the disease/condition).

The present disclosure also provides methods for selecting/stratifying a subject for treatment with a VEGFA-targeted agent. In some embodiments a subject is selected for treatment/prevention in accordance with the present disclosure, or is identified as a subject which would benefit from such treatment/prevention, based on detection/quantification of VEGFA, or of cells comprising/expressing VEGFA, e.g. in a sample obtained from the individual.

Subjects

A subject in accordance with the present disclosure may be any animal. In some embodiments a subject may be mammalian. In some embodiments a subject may be human. In some embodiments a subject may be a non-human animal, e.g. a non-human mammal. The subject may be male or female.

The subject may be a patient. The patient may have a disease/condition described herein. A subject may have been diagnosed with a disease/condition described herein, may be suspected of having a disease/condition described herein, or may be at risk from developing a disease/condition described herein.

A subject/patient may be selected for therapy/prophylaxis in accordance with the present disclosure based on characterisation for markers of a disease/condition described herein.

In some embodiments according to the present disclosure, the subject is preferably a human subject. In some embodiments, the subject to be treated according to a therapeutic or prophylactic method of the present disclosure is a subject having, or at risk of developing, a disease described herein.

Kits

In some aspects of the present disclosure a kit of parts is provided. In some embodiments, the kit may have at least one container having a predetermined quantity of an antigen-binding molecule, nucleic acid, expression vector, cell or composition described herein.

In some embodiments, the kit may comprise materials for producing an antigen-binding molecule, nucleic acid, expression vector, cell or composition described herein.

The kit may provide the antigen-binding molecule, nucleic acid, expression vector, cell or composition together with instructions for administration to a patient in order to treat a specified disease/condition.

Kits according to the present disclosure may include instructions for use, e.g. in the form of an instruction booklet or leaflet. The instructions may include a protocol for performing any one or more of the methods described herein.

Sequence Identity

As used herein, ‘sequence identity’ refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using publicly available computer software such as ClustalOmega (Sdding, J., Bioinformatics (2005) 21, 951-960), T-coffee (Notredame et al., J. Mol. Biol. (2000) 302, 205-217), Kalign (Lassmann and Sonnhammer, BMC Bioinformatics (2005) 6,298) and MAFFT (Katoh and Standley, Molecular Biology and Evolution (2013) 30(4) 772-780) software. When using such software, the default parameters, e.g. for gap penalty and extension penalty, are preferably used.

Sequences

| SEQ ID | | |
|---|---|---|
| NO: | DESCRIPTION | SEQUENCE |
| 1 | 13A6 | SEVQLVESGGGLVQPGGSLRLSCAISGFSLAATDIDWVRQAPGKGLEWVARIFPSAGSTDYA |
| | | DSVKGRFTISADTSKNTVYLQMNSLRAEDTAVYYCGRSDSYSYNVFLHNPTYTALDYRGQGT |
| | | LVTVSS |
| 2 | 13A6, 16A6.1 CDR1 | SLAATD |
| 3 | 13A6 CDR2 | FPSAGSTD |
| 4 | 13A6 CDR3 | GRSDSYSYNVFLHNPTYTALD |
| 5 | 16A2.1 | SEVQLVESGGGLVQPGGSLRLSCAISGFALAETDIDWVRQAPGKGLEWVARIFSSGGNTDYA |
| | | DSVKGRFTISADTSKNTVYLQMNSLRAEDTAVYYCGRSDSIAYNVILPNSTYTARDYRGQGT |
| | | LVTVSS |
| 6 | 16A2.1 CDR1 | ALAETD |
| 7 | 16A2.1 CDR2 | FSSGGNTD |
| 8 | 16A2.1 CDR3 | GRSDSIAYNVILPNSTYTARD |
| 9 | 16A6.1 | SEVQLVESGGGLVQPGGSLRLSCAISGFSLAATDIDWVRQAPGKGLEWVARIFPFSGSTDYT |
| | | DSVKGRFTISADTSKNTVYLQMNSLRAEDTAVYYCGRSDSYSYNVFLHDPAYTALDYRGQGT |
| | | LVTVSS |
| 10 | 16A6.1 CDR2 | FPFSGSTD |
| 11 | 16A6.1 CDR3 | GRSDSYSYNVFLHDPAYTALD |
| 12 | 20A2.1 | SEVQLVESGGGLVQPGGSLRLSCAISGFPLADTDIDWVRQAPGKGLEWVARIFSSYGSTDYA |
| | | DSVKGRFTISADTSKNTVYLQMNSLRAEDTAVYYCGRSDSIAYNIILPASTYSSRDYRGQGT |
| | | LVTVSS |
| 13 | 20A2.1 CDR1 | PLADTD |
| 14 | 20A2.1 CDR2 | FSSYGSTD |
| 15 | 20A2.1 CDR3 | GRSDSIAYNIILPASTYSSRD |
| 16 | 20A3.1 | SEVQLVESGGGLVQPGGSLRLSCAISGFSLADTDIDWVRQAPGKGLEWVARIFSSSGSTYYA |
| | | DSVKGRFTISADTSKNTVYLQMNSLRAEDTAVYYCDRADSVGYNVIFPGSTYSRSGYRGQGT |
| | | LVTVSS |
| 17 | 20A3.1 CDR1 | SLADTD |
| 18 | 20A3.1 CDR2 | FSSSGSTY |
| 19 | 20A3.1 CDR3 | DRADSVGYNVIFPGSTYSRSG |
| 20 | 21A1.1 | SEVQLVESGGGLVQPGGSLRLSCAISGFALATTDIDWVRQAPGKGLEWVARIFNPSDFTDYA |
| | | DSVKGRFTISADTSKNTVYLQMNSLRAEDTAVYYCGRPAVYGYNIFLDSPTYDASQYRGQGT |
| | | LVTVSS |
| 21 | 21A1.1 CDR1 | ALATTD |
| 22 | 21A1.1 CDR2 | FNPSDFTD |
| 23 | 21A1.1 CDR3 | GRPAVYGYNIFLDSPTYDASQ |
| 24 | 21A8.1 | SEVQLVESGGGLVQPGGSLRLSCAISGFSVDATDIDWVRQAPGKGLEWVARIFSPSGFTDYA |
| | | DSVKGRFTISADTSKNTVYLQMNSLRAEDTAVYYCDRPEFYAYNIFHDPRTVPAGNYRGQGT |
| | | LVTVSS |
| 25 | 21A8.1 CDR1 | SVDATD |
| 26 | 21A8.1 CDR2 | FSPSGFTD |
| 27 | 21A8.1 CDR3 | DRPEFYAYNIFHDPRTVPAGN |
| 28 | 21D9.1 | SEVQLVESGGGLVQPGGSLRLSCAISGFSLGATDIDWVRQAPGKGLEWVARIFSPSDFTDYA |
| | | DSVKGRFTISADTSKNTVYLQMNSLRAEDTAVYYCSRPDTYAYNIFPHTASHGAFNYRGQGT |
| | | LVTVSS |
| 29 | 21D9.1 CDR1 | SLGATD |
| 30 | 21D9.1 CDR2 | FSPSDFTD |
| 31 | 21D9.1 CDR3 | SRPDTYAYNIFPHTASHGAFN |
| 32 | 21E6.1 | SEVQLVESGGGLVQPGGSLRLSCAISGFTLAATDIDWVRQAPGKGLEWVARIFTPTGFSDYA |
| | | DSVKGRFTISADTSKNTVYLQMNSLRAEDTAVYYCGIPELYAYNIFMDIPAYNDVYYRGQGT |
| | | LVTVSS |
| 33 | 21E6.1 CDR1 | TLAATD |
| 34 | 21E6.1 CDR2 | FTPTGFSD |
| 35 | 21E6.1 CDR3 | GIPELYAYNIFMDIPAYNDVY |
| 36 | 23D5.1 | SEVQLVESGGGLVQPGGSLRLSCAISGFDLATTDIDWVRQAPGKGLEWVARIFRPSDFTDYA |
| | | DSVKGRFTISADTSKNTVYLQMNSLRAEDTAVYYCVRPDSYAYNILFVTRTFNALHYRGQGT |
| | | LVTVSS |
| 37 | 23D5.1 CDR1 | DLATTD |
| 38 | 23D5.1 CDR2 | FRPSDFTD |
| 39 | 23D5.1 CDR3 | VRPDSYAYNILFVTRTFNALH |
| 40 | FW FR1 | SEVQLVESGGGLVQPGGSLRLSCAISGF |
| 41 | FW_FR2 | IDWVRQAPGKGLEWVARI |
| 42 | FW_FR3_13A6, | YADSVKGRFTISADTSKNTVYLQMNSLRAEDTAVYYC |
| | 16A2.1, 20A2.1, | |
| | 20A3.1, 21A1.1 | |
| | 21A8.1, 21D9.1, | |
| | 21E6.1 | |
| 43 | FW_FR3_16A6.1 | YTDSVKGRFTISADTSKNTVYLQMNSLRAEDTAVYYC |
| 44 | FW FR4 | YRGQGTLVTVSS |
| 45 | CDR1 ConAll | X₁X₂X₃X₄TD wherein X₁ = S, A, P, T or D; X₂ = L or V; X₃ = A, |
| | | D or G; and X₄ = A, D, T or E |
| 46 | CDR2 ConAll | FX₅X₆X₇X₈X₉X₁₀X₁₁ wherein X₅ = S, P, N, T or R; X₆ = P, S or |
| | | F; X₇ = S, Y, G, A or T; X₈ = G or D; X₉ = F, S or N; X₁₀ = |
| | | T or S; and X₁₁ = D or Y |
| 47 | FW_FR3 ConAll | YX₁₂DSVKGRFTISADTSKNTVYLQMNSLRAEDTAVYYC wherein X₁₂ = A or T |
| 48 | CDR3 ConAll | X₁₃X₁₄X₁₅X₁₆X₁₇X₁₈X₁₉YNX₂₀X₂₁X₂₂X₂₃X₂₄X₂₅X₂₆X₂₇X₂₈X₂₉X₃₀X₃₁ |
| | | wherein X₁₃ = G, D, S or V; X₁₄ = R or ₁; X₁₅ = P, S or A; |
| | | X₁₆ = D, A or E; X₁₇ = S, V, L, F or T; X₁₈ = Y, V or I; |
| | | X₁₉ = A, G or S; X₂₀ = ₁ or V; X₂₁ = F, I or L; X₂₂ = L, F M, |
| | | H or P; X₂₃ = D, P, H or V; X₂₄ = N, S, G, A, D, I, P or T; |
| | | X₂₅ = P, S, R or A; X₂₆ = T, A or S; X₂₇ = Y, V, H or F; |
| | | X₂₈ = T, D, S, N, P or G; X₂₉ = A, S, R or D; X₃₀ = S, L, R, |
| | | V, G or F; and X₃₁ = D, Q, N, G, Y or H |
| 49 | Con SeqAll | SEVQLVESGGGLVQPGGSLRLSCAISGFX₁X₂X₃X₄TDIDWVRQAPGKGLEWVARIFX₅X₆ |
| | | X₇X₈X₉X₁₀X₁₁YX₁₂DSVKGRFTISADTSKNTVYLQMNSLRAEDTAVYYCX₁₃X₁₄X₁₅ |
| | | X₁₆X₁₇X₁₈X₁₉YNX₂₀X₂₁X₂₂X₂₃X₂₄X₂₅X₂₆X₂₇X₂₈X₂₉X₃₀X₃₁YRGQGTLVTVS |
| | | S wherein X₁ = S, A, P, T or D; X₂ = L or V; X₃ = A, D or G; |
| | | X₄ = A, D, T or E; X₅ = S, P, N, T or R; X₆ = P, S or F; |
| | | X₇ = S, Y, G, A or T; X₈ = G or D; X₉ = F, S or N; X₁₀ = T or |
| | | S; X₁₁ = D or Y; X₁₂ = A or T; X₁₃ = G, D, S or V; X₁₄ = R or |
| | | ₁; X₁₅ = P, S or A; X₁₆ = D, A or E; X₁₇ = S, V, L, F or T; |
| | | X₁₈ = Y, Vor l; X₁₉ = A, G or S; X₂₀ = ₁ or V; X₂₁ = F, I or |
| | | L; X₂₂ = L, F M, H or P; X₂₃ = D, P, H or V; X₂₄ = N, S, G, |
| | | A, D, I, P or T; X₂₅ = P, S, R or A; X₂₆ = T, A or S; X₂₇ = |
| | | Y, V, H or F; X₂₈ = T, D, S, N, P or G; X₂₉ = A, S, R or D; |
| | | X₃₀ = S, L, R, V, G or F; and X₃₁ = D, Q, N, G, Y or H |
| 50 | CDR1 | X₃₂LAX₃₃TD wherein X₃₂ = P, S or A; and X₃₃ = D or E |
| | Con16A2.1derived | |
| 51 | CDR2 | FSSX₃₄GX₃₅TX₃₆ wherein X₃₄ = Y, S or G; X₃₅ = S or N; and |
| | Con16A2.1derived | X₃₆ = D or Y |
| 52 | CDR3 | X₃₇RX₃₈DSX₃₉X₄₀YNX₄₁₁X₄₂PX₄₃STYX₄₄X₄₅X₄₆X₄₇ wherein X₃₇ = G or |
| | Con16A2.1derived | D; X₃₈ = S or A; X₃₉ = I or V; X₄₀ = A or G; X₄₁ = V or |
| | | I; X₄₂ = L or F; X₄₃ = G, A or N; X₄₄ = S or T; X₄₅ = R, |
| | | S or A; X₄₆ = R or S; and X₄₇ = D or G |
| 53 | Con Seq16A2.1derived | SEVQLVESGGGLVQPGGSLRLSCAISGFX₃₂LAX₃₃TDIDWVRQAPGKGLEWVARIFSSX₃₄ |
| | | GX₃₅TX₃₆YADSVKGRFTISADTSKNTVYLQMNSLRAEDTAVYYCX₃₇RX₃₈DSX₃₉X₄₀YN |
| | | X₄₁₁X₄₂PX₄₃STYX₄₄X₄₅X₄₆X₄₇YRGQGTLVTVSS |
| | | wherein X₃₂ = P, S or A; X₃₃ = D or E; X₃₄ = Y, S or G; X₃₅ = |
| | | S or N; X₃₆ = D or Y; X₃₇ = G or D; X₃₈ = S or A; X₃₉ = I or |
| | | V; X₄₀ = A or G; X₄₁ = V or I; X₄₂ = L or F; X₄₃ = G, A or N; |
| | | X₄₄ = S or T; X₄₅ = R, S or A; X₄₆ = R or S; and X₄₇ = D or G |
| 54 | CDR1 Con21/23 | X₄₈X₄₉X₅₀X₅₁TD wherein X₄₈ = A, S, T or D; X₄₉ = L or V; X₅₀ = |
| | | A, D or G; and X₅₁ = A or T |
| 55 | CDR2 Con21/23 | FX₅₂PX₅₃X₅₄FX₅₅D wherein X₅₂ = S, N or T; X₅₃ = S or T; X₅₄ = |
| | | D or G; and X₅₅ = T or S |
| 56 | CDR3 Con21/23 | X₅₆X₅₇PX₅₈X₅₉YX₆₀YNIX₆₁X₆₂X₆₃X₆₄X₆₅X₆X₆₇X₆₈X₆₉X₇₀X₇₁ wherein |
| | | X₅₆ = G, D, S or V; X₅₇ = R or ₁; X₅₈ = A, E or D; X₅₉ = V, T, |
| | | L, S or F; X₆₀ = A or G; X₆₁ = F or L; X₆₂ = L, H, P or M; |
| | | X₆₃ = D, H or V; X₆₄ = S, R, P or I; X₆₅ = P, R or A; X₆₆ = T, |
| | | S or A; X₆₇ = Y, V, Hor F; X₆₈ = D, N, G or P; X₆₉ = A or D; |
| | | X₇₀ = S, G, F, V or L; and X₇₁ = Q, N, Y or H |
| 57 | Con Seq21/23 | SEVQLVESGGGLVQPGGSLRLSCAISGFX₄₈X₄₉X₅₀X₅₁TDIDWVRQAPGKGLEWVARIF |
| | | X₅₂PX₅₃X₅₄FX₅₅DYADSVKGRFTISADTSKNTVYLQMNSLRAEDTAVYYCX₅₆X₅₇PX₅₈ |
| | | X₅₉YX₆₀YNIX₆₁X₆₂X₆₃X₆₄X₆₅X₆₆X₆₇X₆₈X₆₉X₇₀X₇₁YRGQGTLVTVSS |
| | | wherein X₄₈ = A, S, T or D; X₄₉ = L or V; X₅₀ = A, D or G; |
| | | X₅₁ = A or T; X₅₂ = S, N or T; X₅₃ = S or T; X₅₄ = D or G; |
| | | X₅₅ = T or S; X₅₆ = G, D, S or V; X₅₇ = R or I; X₅₈ = A, E or |
| | | D; X₅₉ = V, T, L, S or F; X₆₀ = A or G; X₆₁ = F or L; X₆₂ = L, |
| | | H, P or M; X₆₃ = D, Hor V; X₆₄ = S, R, P or I; X₆₅ = P, R or |
| | | A; X₆₆ = T, S or A; X₆₇ = Y, V, Hor F; X₆₈ = D, N, G or P; |
| | | X₆₉ = A or D; X₇₀ = S, G, F, V or L; and X₇₁ = Q, N, Y or H |
| 58 | GS linker | GGGGSGGGGSGGGGSGGGGSGGGGSGGGGSGGGG |
| 59 | 16A2.1x2 | SEVQLVESGGGLVQPGGSLRLSCAISGFALAETDIDWVRQAPGKGLEWVARIFSSGGNTDYA |
| | | DSVKGRFTISADTSKNTVYLQMNSLRAEDTAVYYCGRSDSIAYNVILPNSTYTARDYRGQGT |
| | | LVTVSSGGGGSGGGGSGGGGSGGGGSGGGGSGGGGSGGGGSEVQLVESGGGLVQPGGSLRLS |
| | | CAISGFALAETDIDWVRQAPGKGLEWVARIFSSGGNTDYADSVKGRFTISADTSKNTVYLQM |
| | | NSLRAEDTAVYYCGRSDSIAYNVILPNSTYTARDYRGQGTLVTVSS |
| 60 | VEGF206 | MNFLLSWVHWSLALLLYLHHAKWSQAAPMAEGGGQNHHEVVKFMDVYQRSYCHPIETLVDIF |
| | | QEYPDEIEYIFKPSCVPLMRCGGCCNDEGLECVPTEESNITMQIMRIKPHQGQHIGEMSFLQ |
| | | HNKCECRPKKDRARQEKKSVRGKGKGQKRKRKKSRYKSWSVYVGARCCLMPWSLPGPHPCGP |
| | | CSERRKHLFVQDPQTCKCSCKNTDSRCKARQLELNERTCRCDKPRR |
| 61 | VEGF189 | MNFLLSWVHWSLALLLYLHHAKWSQAAPMAEGGGQNHHEVVKFMDVYQRSYCHPIETLVDIF |
| | | QEYPDEIEYIFKPSCVPLMRCGGCCNDEGLECVPTEESNITMQIMRIKPHQGQHIGEMSFLQ |
| | | HNKCECRPKKDRARQEKKSVRGKGKGQKRKRKKSRYKSWSVPCGPCSERRKHLFVQDPQTCK |
| | | CSCKNTDSRCKARQLELNERTCRCDKPRR |
| 62 | VEGF165 | MNFLLSWVHWSLALLLYLHHAKWSQAAPMAEGGGQNHHEVVKFMDVYQRSYCHPIETLVDIF |
| | | QEYPDEIEYIFKPSCVPLMRCGGCCNDEGLECVPTEESNITMQIMRIKPHQGQHIGEMSFLQ |
| | | HNKCECRPKKDRARQENPCGPCSERRKHLFVQDPQTCKCSCKNTDSRCKARQLELNERTCRC |
| | | DKPRR |
| 63 | VEGF121 | MNFLLSWVHWSLALLLYLHHAKWSQAAPMAEGGGQNHHEVVKFMDVYQRSYCHPIETLVDIF |
| | | QEYPDEIEYIFKPSCVPLMRCGGCCNDEGLECVPTEESNITMQIMRIKPHQGQHIGEMSFLQ |
| | | HNKCECRPKKDRARQEKCDKPRR |
| 64 | Mature VEGF206 | APMAEGGGQNHHEVVKFMDVYQRSYCHPIETLVDIFQEYPDEIEYIFKPSCVPLMRCGGCCN |
| | | DEGLECVPTEESNITMQIMRIKPHQGQHIGEMSFLQHNKCECRPKKDRARQEKKSVRGKGKG |
| | | QKRKRKKSRYKSWSVYVGARCCLMPWSLPGPHPCGPCSERRKHLFVQDPQTCKCSCKNTD |
| | | SRCKARQLELNERTCRCDKPRR |
| 65 | Mature VEGF189 | APMAEGGGQNHHEVVKFMDVYQRSYCHPIETLVDIFQEYPDEIEYIFKPSCVPLMRCGGCCN |
| | | DEGLECVPTEESNITMQIMRIKPHQGQHIGEMSFLQHNKCECRPKKDRARQEKKSVRGKGKG |
| | | QKRKRKKSRYKSWSVPCGPCSERRKHLFVQDPQTCKCSCKNTDSRCKARQLELNERTCRC |
| | | DKPRR |
| 66 | Mature VEGF165 | APMAEGGGQNHHEVVKFMDVYQRSYCHPIETLVDIFQEYPDEIEYIFKPSCVPLMRCGGCCN |
| | | DEGLECVPTEESNITMQIMRIKPHQGQHIGEMSFLQHNKCECRPKKDRARQENPCGPCSERR |
| | | KHLFVQDPQTCKCSCKNTDSRCKARQLELNERTCRCDKPRR |
| 67 | Mature VEGF121 | APMAEGGGQNHHEVVKFMDVYQRSYCHPIETLVDIFQEYPDEIEYIFKPSCVPLMRCGGCCN |
| | | DEGLECVPTEESNITMQIMRIKPHQGQHIGEMSFLQHNKCECRPKKDRARQEKCDKPRR |
| 68 | VEGFA Signal Peptide | MNFLLSWVHWSLALLLYLHHAKWSQA |
| 69 | Human IgG1 constant | ASTKGPSVFPLAPSSKSTSGGTAALGCLVKDYFPEPVTVSWNSGALTSGVHTFPAVLQSSGL |
| | region (IGHG1; | YSLSSVVTVPSSSLGTQTYICNVNHKPSNTKVDKKVEPKSCDKTHTCPPCPAPELLGGPSVF |
| | UniProt:P01857-1, v1) | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSRDELTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 70 | CH1 lgG1 (positions | ASTKGPSVFPLAPSSKSTSGGTAALGCLVKDYFPEPVTVSWNSGALTSGVHTFPAVLQSSGL |
| | 1-98 of P01857-1, v1) | YSLSSVVTVPSSSLGTQTYICNVNHKPSNTKVDKKV |
| 71 | Hinge IgG1 (positions | EPKSCDKTHTCP |
| | 99-110 of P01857-1, | |
| | v1) | |
| 72 | CH2 lgG1 (positions | PCPAPELLGGPSVFLFPPKPKDTLMISRTPEVTCVVVDVSHEDPEVKFNWYVDGVEVHNAKT |
| | 111-223 of P01857-1, | KPREEQYNSTYRVVSVLTVLHQDWLNGKEYKCKVSNKALPAPIEKTISKAK |
| | v1) | |
| 73 | CH3 lgG1 (positions | GQPREPQVYTLPPSRDELTKNQVSLTCLVKGFYPSDIAVEWESNGQPENNYKTTPPVLDSDG |
| | 224-330 of P01857-1, | SFFLYSKLTVDKSRWQQGNVFSCSVMHEALHNHYTQKSLSLSPGK |
| | v1) | |
| 74 | Ranibizumab Heavy | EVQLVESGGGLVQPGGSLRLSCAASGYDFTHYGMNWVRQAPGKGLEWVGWINTYTGEPTYAA |
| | Chain | DFKRRFTFSLDTSKSTAYLQMNSLRAEDTAVYYCAKYPYYYGTSHWYFDVWGQGTLVTVSSA |
| | | STKGPSVFPLAPSSKSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHL |
| 75 | Ranibizumab Light | DIQLTQSPSSLSASVGDRVTITCSASQDISNYLNWYQQKPGKAPKVLIYFTSSLHSGVPSRF |
| | Chain | SGSGSGTDFTLTISSLQPEDFATYYCQQYSTVPWTFGQGTKVEIKRTVAAPSVFIFPPSDEQ |
| | | LKSGTASVVCLLNNFYPREAKVQWKVDNALQSGNSQESVTEQDSKDSTYSLSSTLTLSKADY |
| | | EKHKVYACEVTHQGLSSPVTKSFNRGEC |
| 76 | Bevacizumab Heavy | EVQLVESGGGLVQPGGSLRLSCAASGYTFTNYGMNWVRQAPGKGLEWVGWINTYTGEPTYAA |
| | Chain | DFKRRFTFSLDTSKSTAYLQMNSLRAEDTAVYYCAKYPHYYGSSHWYFDVWGQGTLVTVSSA |
| | | STKGPSVFPLAPSSKSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPCPAPELLGGPSVFL |
| | | FPPKPKDTLMISRTPEVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYTLPPSREEMTKNQVSLT |
| | | CLVKGFYPSDIAVEWESNGQPENNYKTTPPVLDSDGSFFLYSKLTVDKSRWQQGNVFSCSVM |
| | | HEALHNHYTQKSLSLSPGK |
| 77 | Bevacizumab Light | DIQMTQSPSSLSASVGDRVTITCSASQDISNYLNWYQQKPGKAPKVLIYFTSSLHSGVPSRF |
| | Chain | SGSGSGTDFTLTISSLQPEDFATYYCQQYSTVPWTFGQGTKVEIKRTVAAPSVFIFPPSDEQ |
| | | LKSGTASVVCLLNNFYPREAKVQWKVDNALQSGNSQESVTEQDSKDSTYSLSSTLTLSKADY |
| | | EKHKVYACEVTHQGLSSPVTKSFNRGEC |

The present disclosure includes the combination of the aspects and preferred features described except where such a combination is clearly impermissible or expressly avoided.

The section headings used herein are for organisational purposes only and are not to be construed as limiting the subject matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word ‘comprise,’ and variations such as ‘comprises’ and ‘comprising,’ will be understood to imply the inclusion of a stated integer or step or group of integers or steps but not the exclusion of any other integer or step or group of integers or steps.

It must be noted that, as used in the specification and the appended claims, the singular forms ‘a,’ ‘an,’ and ‘the’ include plural referents unless the context clearly dictates otherwise. Ranges may be expressed herein as from ‘about’ one particular value, and/or to ‘about’ another particular value. When such a range is expressed, another embodiment includes from the one particular value and/or to the other particular value. Similarly, when values are expressed as approximations, by the use of the antecedent ‘about,’ it will be understood that the particular value forms another embodiment.

Where a nucleic acid sequence is disclosed herein, the reverse complement thereof is also expressly contemplated.

Methods described herein may preferably be performed in vitro. The term ‘in vitro’ is intended to encompass procedures performed with cells in culture whereas the term ‘in vivo’ is intended to encompass procedures with/on intact multi-cellular organisms.

### BRIEF DESCRIPTION OF THE FIGURES

Embodiments and experiments illustrating the principles of the present disclosure will now be discussed with reference to the accompanying figures.

FIG. 1. Tables summarising amino acid differences in CDR1, CDR2 and CDR3 regions of Library 1 and Library 2, 4D5 (trastuzumab) and the starting template. “Xaa” denotes randomized amino acid.

FIGS. 2A to 2K. Sensorgrams showing binding of (2A) 13A6, (2B) 16A2.1, (2C) 16A6.1 and (2D) 16A2.1x2, (2E) 20A2.1, (2F) 20A3.1, (2G) 21A1.1, (2H) 21A8.1, (2I) 21D9.1, (2J) 21E6.1 and (2K) 23D5.1 to human VEGFA, as measured by biolayer interferometry (BLI). The concentrations tested for each DotBody are stated below the binding curves, in nM.

FIGS. 3A to 3J. Sensorgrams showing binding of (3A) 13A6, (3B) 16A2.1, (3C) 16A6.1, (3D) 20A2.1, (3E) 20A3.1, (3F) 21A1.1, (3G) 21A8.1, (3H) 21D9.1, (31) 21E6.1 and (3J) 23D5.1 to mouse VEGFA, as measured by biolayer interferometry (BLI). The concentrations tested for each DotBody are stated below the binding curves, in nM.

FIG. 4. Graph showing inhibition of the interaction between human VEGFA and VEGFR1 by 13A6, 16A2.1, 16A6.1 and Ranibizumab, in competitive ELISA.

FIGS. 5A to 51. Graphs showing inhibition of the interaction between human VEGFA and VEGFR1 by (5A) 16A2.1, (5B) 20A2.1, (5C) 20A3.1, (5D) 21A1.1, (5E) 21A8.1, (5F) 21D9.1, (5G) 21E6.1, (5H) 23D5.1 and (5I) Ranibizumab, in competitive ELISA.

FIGS. 6A to 6H. Sensorgrams showing binding of anti-VEGFA DotBodies (6A) 16A2.1, (6B) 20A2.1, (6C) 20A3.1, (6D) 21A1.1, (6E) 21A8.1, (6F) 21D9.1, (6G) 21E9.1 and (6H) 23D5.1 to human VEGFA, at a single concentration of 250 nM, after incubation for 1 h at room temperature, 60° C., 70° C. or 80° C. Measurements were performed by BLI as described in Example 10.

### EXAMPLES

### Example 1: Generation of Naïve Synthetic DotBody Phage Display Libraries

Two DotBody phage display libraries were used for the identification of anti-VEGF DotBodies. These libraries were based on a humanized, stabilized and autonomous VH domain template derived from the trastuzumab VH domain (“DotBody scaffold patents”, described e.g. in WO 2016/072938A1).

Library 1 was based on the following VH domain template sequence (positions mutated for library creation are underlined):

SEVQLVESGGGLVQPGGSLRLSSAISGFSISSTSIDWVRQAPGKGLEWVA
RISPSSGSTSYADSVKGRFTISADTSKNTVYLQMNSLRAEDTAVYYTGRS
SSAMDYRGQGTLVTVSS

Library 2 was based on the following VH domain template sequence (positions mutated for library creation are underlined):

SEVQLVESGGGLVQPGGSLRLSCAISGFSISSTSIDWVRQAPGKGLEWVA
RISPSSGSTSYADSVKGRFTISADTSKNTVYLQMNSLRAEDTAVYYCGRS
SSAMDYRGQGTLVTVSS

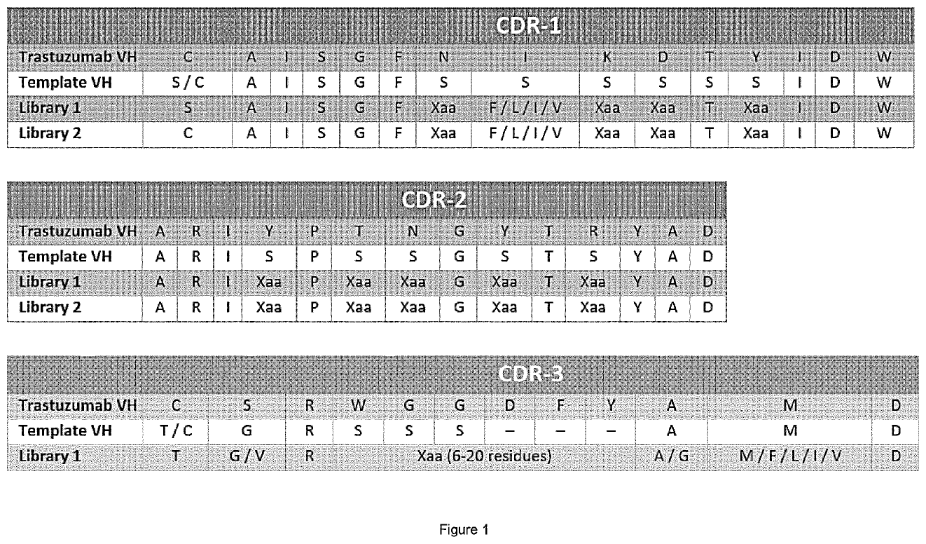

CDR-1, CDR-2 and CDR-3 of the VH domain template were randomized according to the design shown in FIG. 1, by Kunkel mutagenesis according to procedures by Bostrom J. et al. (14,15) and Tonikian R. et al. (16). The primers employed for Library 1 are shown in Table 1, while those employed for Library 2 are shown in Table 2.

Library 1 contained approximately 2.87×10¹⁰ clones, while Library 2 contained approximately 1.37×10¹⁰ clones with all CDRs mutated. The libraries were assessed by serial dilution and colony counting after library transformation.

TABLE 1
Primers employed to create Library 1. Primer
H1 and H2 were mixed with primers H3.6-H3.20
before performing Kunkel mutagenesis on the VH
template sequence, to create fifteen sub-
libraries that were later combined to form the
final library. Codon [XYZ] have nucleotide
frequencies as follows: X = 0.2G + 0.2A +
0.5T + 0.1C, Y = 0.4A + 0.2T + 0.4C and Z =
0.1G + 0.9C.

| Primer | |
|---|---|
| name | Primer Sequence |
| H1 | CCTCTGCAATTTCTGGCTTC[XYZ]NTT[XYZ][XYZ]ACT |
| | [XYZ]ATAGACTGGGTGCGTCAGG |
| H2 | CTGGAATGGGTTGCAAGGATT[XYZ]CCT[XYZ][XYZ]GG |
| | T[XYZ]ACT[XYZ]TATGCCGATAGCGTCAAGGG |
| H3.6 | GCCGTCTATTATACTGKCCGC[XYZ][XYZ][XYZ][XYZ] |
| | [XYZ][XYZ]GSTNTKGACTACCGGGGTCAAG |
| H3.7 | GCCGTCTATTATACTGKCCGC[XYZ][XYZ][XYZ][XYZ] |
| | [XYZ][XYZ][XYZ]GSTNTKGACTACCGGGGTCAAG |
| H3.8 | GCCGTCTATTATACTGKCCGC[XYZ][XYZ][XYZ][XYZ] |
| | [XYZ][XYZ][XYZ][XYZ]GSTNTKGACTACCGGGGTCAA |
| | G |
| H3.9 | GCCGTCTATTATACTGKCCGC[XYZ][XYZ][XYZ][XYZ] |
| | [XYZ][XYZ][XYZ][XYZ][XYZ]GSTNTKGACTACCGGG |
| | GTCAAG |
| H3.10 | GCCGTCTATTATACTGKCCGC[XYZ][XYZ][XYZ][XYZ] |
| | [XYZ][XYZ][XYZ][XYZ][XYZ][XYZ]GSTNTKGACTA |
| | CCGGGGTCAAG |
| H3.11 | GCCGTCTATTATACTGKCCGC[XYZ][XYZ][XYZ][XYZ] |
| | [XYZ][XYZ][XYZ][XYZ][XYZ][XYZ][XYZ]GSTNTK |
| | GACTACCGGGGTCAAG |
| H3.12 | GCCGTCTATTATACTGKCCGC[XYZ][XYZ][XYZ][XYZ] |
| | [XYZ][XYZ][XYZ][XYZ][XYZ][XYZ][XYZ][XYZ]G |
| | STNTKGACTACCGGGGTCAAG |
| H3.13 | GCCGTCTATTATACTGKCCGC[XYZ][XYZ][XYZ][XYZ] |
| | [XYZ][XYZ][XYZ][XYZ][XYZ][XYZ][XYZ][XYZ] |
| | [XYZ]GSTNTKGACTACCGGGGTCAAG |
| H3.14 | GCCGTCTATTATACTGKCCGC[XYZ][XYZ][XYZ][XYZ] |
| | [XYZ][XYZ][XYZ][XYZ][XYZ][XYZ][XYZ][XYZ] |
| | [XYZ][XYZ]GSTNTKGACTACCGGGGTCAAG |
| H3.15 | GCCGTCTATTATACTGKCCGC[XYZ][XYZ][XYZ][XYZ] |
| | [XYZ][XYZ][XYZ][XYZ][XYZ][XYZ][XYZ][XYZ] |
| | [XYZ][XYZ][XYZ]GSTNTKGACTACCGGGGTCAAG |
| H3.16 | GCCGTCTATTATACTGKCCGC[XYZ][XYZ][XYZ][XYZ] |
| | [XYZ][XYZ][XYZ][XYZ][XYZ][XYZ][XYZ][XYZ] |
| | [XYZ][XYZ][XYZ][XYZ]GSTNTKGACTACCGGGGTCAA |
| | G |
| H3.17 | GCCGTCTATTATACTGKCCGC[XYZ][XYZ][XYZ][XYZ] |
| | [XYZ][XYZ][XYZ][XYZ][XYZ][XYZ][XYZ][XYZ] |
| | [XYZ][XYZ][XYZ][XYZ][XYZ]GSTNTKGACTACCGGG |
| | GTCAAG |
| H3.18 | GCCGTCTATTATACTGKCCGC[XYZ][XYZ][XYZ][XYZ] |
| | [XYZ][XYZ][XYZ][XYZ][XYZ][XYZ][XYZ][XYZ] |
| | [XYZ][XYZ][XYZ][XYZ][XYZ][XYZ]GSTNTKGACTA |
| | CCGGGGTCAAG |
| H3.19 | GCCGTCTATTATACTGKCCGC[XYZ][XYZ][XYZ][XYZ] |
| | [XYZ][XYZ][XYZ][XYZ][XYZ][XYZ][XYZ][XYZ] |
| | [XYZ][XYZ][XYZ][XYZ][XYZ][XYZ][XYZ]GSTNTK |
| | GACTACCGGGGTCAAG |
| H3.20 | GCCGTCTATTATACTGKCCGC[XYZ][XYZ][XYZ][XYZ] |
| | [XYZ][XYZ][XYZ][XYZ][XYZ][XYZ][XYZ][XYZ] |
| | [XYZ][XYZ][XYZ][XYZ][XYZ][XYZ][XYZ][XYZ]G |
| | STNTKGACTACCGGGGTCAAG |

TABLE 2
Primers employed to create Library 2. Primer
H1 and H2 were mixed with primers H3.6-H3.20
before performing Kunkel mutagenesis on the
VH template sequence, to create fifteen sub-
libraries that were later combined to form
the final library. Codon [XYZ] have nucleotide
frequencies as follows: X = 0.2G + 0.2A +
0.5T + 0.1C, Y = 0.4A + 0.2T + 0.4C and Z =
0.1G + 0.9C.

| Primer | |
|---|---|
| name | Primer sequence |
| H1 | CCTGTGCAATTTCTGGCTTC[XYZ]NTT[XYZ][XYZ]ACT |
| | [XYZ]ATAGACTGGGTGCGTCAGG |
| H2 | CTGGAATGGGTTGCAAGGATT[XYZ]CCT[XYZ][XYZ]GG |
| | T[XYZ]ACT[XYZ]TATGCCGATAGCGTCAAGGG |
| H3.6 | GCCGTCTATTATTGTGKCCGC[XYZ][XYZ][XYZ][XYZ] |
| | [XYZ][XYZ]GSTNTKGACTACCGGGGTCAAG |
| H3.7 | GCCGTCTATTATTGTGKCCGC[XYZ][XYZ][XYZ][XYZ] |
| | [XYZ][XYZ][XYZ]GSTNTKGACTACCGGGGTCAAG |
| H3.8 | GCCGTCTATTATTGTGKCCGC[XYZ][XYZ][XYZ][XYZ] |
| | [XYZ][XYZ][XYZ][XYZ]GSTNTKGACTACCGGGGTCAA |
| | G |
| H3.9 | GCCGTCTATTATTGTGKCCGC[XYZ][XYZ][XYZ][XYZ] |
| | [XYZ][XYZ][XYZ][XYZ][XYZ]GSTNTKGACTACCGGG |
| | GTCAAG |
| H3.10 | GCCGTCTATTATTGTGKCCGC[XYZ][XYZ][XYZ][XYZ] |
| | [XYZ][XYZ][XYZ][XYZ][XYZ][XYZ]GSTNTKGACTA |
| | CCGGGGTCAAG |
| H3.11 | GCCGTCTATTATTGTGKCCGC[XYZ][XYZ][XYZ][XYZ] |
| | [XYZ][XYZ][XYZ][XYZ][XYZ][XYZ][XYZ]GSTNTK |
| | GACTACCGGGGTCAAG |
| H3.12 | GCCGTCTATTATTGTGKCCGC[XYZ][XYZ][XYZ][XYZ] |
| | [XYZ][XYZ][XYZ][XYZ][XYZ][XYZ][XYZ][XYZ]G |
| | STNTKGACTACCGGGGTCAAG |
| H3.13 | GCCGTCTATTATTGTGKCCGC[XYZ][XYZ][XYZ][XYZ] |
| | [XYZ][XYZ][XYZ][XYZ][XYZ][XYZ][XYZ][XYZ] |
| | [XYZ]GSTNTKGACTACCGGGGTCAAG |
| H3.14 | GCCGTCTATTATTGTGKCCGC[XYZ][XYZ][XYZ][XYZ] |
| | [XYZ][XYZ][XYZ][XYZ][XYZ][XYZ][XYZ][XYZ] |
| | [XYZ][XYZ]GSTNTKGACTACCGGGGTCAAG |
| H3.15 | GCCGTCTATTATTGTGKCCGC[XYZ][XYZ][XYZ][XYZ] |
| | [XYZ][XYZ][XYZ][XYZ][XYZ][XYZ][XYZ][XYZ] |
| | [XYZ][XYZ][XYZ]GSTNTKGACTACCGGGGTCAAG |
| H3.16 | GCCGTCTATTATTGTGKCCGC[XYZ][XYZ][XYZ][XYZ] |
| | [XYZ][XYZ][XYZ][XYZ][XYZ][XYZ][XYZ][XYZ] |
| | [XYZ][XYZ][XYZ][XYZ]GSTNTKGACTACCGGGGTCAA |
| | G |
| H3.17 | GCCGTCTATTATTGTGKCCGC[XYZ][XYZ][XYZ][XYZ] |
| | [XYZ][XYZ][XYZ][XYZ][XYZ][XYZ][XYZ][XYZ] |
| | [XYZ][XYZ][XYZ][XYZ][XYZ]GSTNTKGACTACCGGG |
| | GTCAAG |
| H3.18 | GCCGTCTATTATTGTGKCCGC[XYZ][XYZ][XYZ][XYZ] |
| | [XYZ][XYZ][XYZ][XYZ][XYZ][XYZ][XYZ][XYZ] |
| | [XYZ][XYZ][XYZ][XYZ][XYZ][XYZ]GSTNTKGACTA |
| | CCGGGGTCAAG |
| H3.19 | GCCGTCTATTATTGTGKCCGC[XYZ][XYZ][XYZ][XYZ] |
| | [XYZ][XYZ][XYZ][XYZ][XYZ][XYZ][XYZ][XYZ] |
| | [XYZ][XYZ][XYZ][XYZ][XYZ][XYZ][XYZ]GSTNTK |
| | GACTACCGGGGTCAAG |
| H3.20 | GCCGTCTATTATTGTGKCCGC[XYZ][XYZ][XYZ][XYZ] |
| | [XYZ][XYZ][XYZ][XYZ][XYZ][XYZ][XYZ][XYZ] |
| | [XYZ][XYZ][XYZ][XYZ][XYZ][XYZ][XYZ][XYZ]G |
| | STNTKGACTACCGGGGTCAAG |

### Example 2: Phage Display Selections from Naïve Synthetic DotBody Phage Display Libraries

Human VEGF-121 (Acro Biosystems) was immobilized onto Maxisorp Immuno Tubes (Thermo Scientific) at 20 μg in 1 mL PBS for round 1, and 10 μg in 1 mL PBS for subsequent rounds, overnight at 4° C. The tubes were washed twice in PBS, and blocked for 1 h at room temperature (RT) in Milk Block Buffer (MBB: 1% skimmed milk in PBST i.e. PBS containing 0.05% Tween-20). Negative selection tubes were prepared in the same way as described for VEGF-121, but PBS was used in place of the VEGF-121 protein. 500 μL of library 1 and library 2 (at ˜2×10¹³ pfu/mL) were precipitated with PEG/NaCl buffer (20% PEG 8,000, 2.5M NaCl), and resuspended in MBB, before being transferred to the negative selection tube for incubation for 1 h at RT. The phages were transferred to the immuno tube coated with VEGF-121 and incubated for 2 h at RT. The tube was then washed 3 times with MBB, 3 times with PBST and twice with PBS, to remove non-bound phages. The bound phages were eluted with trypsin at 1 mg/mL in trypsin buffer (TBS+2 mM CaCl₂). The eluted phages were used to infect 5 mL of TG1 bacterial cell culture (in 2YT media) in exponential growth phase (OD₆₀₀ 0.5) for 30 min at 37° C. From round 2 onwards, 1.2 mL of infected TG1 cells were stored with 20% glycerol at −80° C., to be used for monoclonal screening (glycerol stocks for monoclonal screening). The remaining infected TG1 cells were transferred to 50 mL 2YT. The culture was incubated at 37° C. with shaking until OD₆₀₀˜0.5, before infection with 1×10¹⁰ pfu/mL M13K07 helper phage for 30 min at 37° C. The infected TG1 cells were pelleted at 3,900 g for 20 min at 4° C., resuspended in 500 μL 2YT broth and plated onto 2×15 cm round 2YT agar plates supplemented with 100 μg/mL carbenicillin and 50 μg/mL kanamycin. After overnight incubation at 30° C., the bacterial-lawn was resuspended in 25 mL TBS. The phage produced were purified by precipitation with PEG/NaCl buffer. After two rounds of PEG/NaCl precipitation, the phages were resuspended in PBS+10% glycerol. The purified phages were used for the subsequent round of selection.

Four rounds of selections were performed against VEGF-121. From the second selection round onwards, the number of washes was increased as follows:
Round 2: 4 times with MBB, 4 times with PBST, and 2 times with PBS.
Round 3: 6 times with MBB, 6 times with PBST, 2 times with PBS
Round 4: 7× with MBB, 7 times with PBST, 2 times with PBS From the second selection round onwards, 1 mL of phages purified from the previous round of selection at a final concentration of 1×10¹² pfu/mL was used. The rest of the panning procedure was identical.

### Example 3: Identification of Unique Binders by Monoclonal Phage ELISA

Monoclonal phage ELISA was used to identify unique binding DotBodies selected from the naïve libraries, as well as the affinity maturation library. The glycerol stocks for monoclonal screening were plated onto 2YT agar plates supplemented with 100 μg/mL Carbenicillin and incubated overnight at 37° C. Individual colonies were grown in 1 mL 2YT broth supplemented with 100 μg/mL Carbenicillin for 2 hours before infection with 1×10¹⁰ pfu/mL M13K07 helper phage. The cultures were further supplemented with 50 μg/mL Kanamycin and incubated at 30° C. overnight.

The cells were pelleted by centrifugation at 1,100 g for 10 mins at 4° C., and the supernatant was used for phage monoclonal ELISA. In the phage monoclonal ELISA, VEGF-121 was immobilized at a concentration of 1 μg/mL onto a Maxisorp 96-wells plate (Thermo Scientific) overnight at 4° C., washed twice with PBS and blocked for 1 h at RT with MBB. 25 μL of the phage culture supernatant was mixed with 25 μL of MBB, added to the plate and incubated for 2 h at RT. The plate was washed 8 times with PBST before 50 μL of anti-M13 antibody HRP conjugate (GE Healthcare) was added at a 1:7,000 dilution in MBB, and incubated for 1 h at RT. The plate was washed 8 times with PBST, and developed with 50 μL 3,3′,5,5′-Tetramethylbenzidine (TMB) substrate (GeneTex). After 5-15 min, the reactions were stopped by adding 50 μL of 2M H₂SO₄, and the signal was measured at an absorbance of 450 nm. Monoclonal clones with high signal intensity (Absorbance higher than 1) were sequenced by Sanger sequencing to identify unique VH domains binding to VEGF-121.

### Example 4: Affinity Maturation Phage Display Library Construction

Anti-VEGF DotBody 13A6 was selected for affinity maturation, as its binding affinity is below 50 nM and it also blocked the interaction between VEGFA and the VEGF receptor 1 (VEGFR1). An affinity maturation phage display library was created by Kunkel mutagenesis, according to Bostrom J. et al. (14). by using the primers shown in Table 3. The library contained 1.1×10⁸ unique sequences, as estimated by serial dilutions upon library electroporation into TG1 cells, plating onto 2YT agar supplemented with 100 μg/mL Carbenicillin, and subsequent sequencing of plasmids from 30 colonies.

TABLE 3
DNA sequences of primers employed to generate
an affinity maturation phage display library
of anti-VEGF DotBody 13A6. The numbers in the
sequence represent frequency of bases A, T, C
and G in the oligonucleotide sequence.
Frequency code 5 = 70% A, 10% G, 10% C,
10% T; 6 = 70% G, 10% A, 10% C, 10% T; 7 =
70% C, 10% A, 10% G, 10% T; 8 = 70% T, 10% A,
10% G, 10% C.

| Primer | |
|---|---|
| name | Primer sequence |
| aVEGF- | TGTGCAATTTCTGGCTTC878788678678577657ATAGA |
| 13A6 | CTGGGTGCGTCAG |
| CDR1 | |
| aVEGF- | GAATGGGTTGCAAGGATT88877687867866887857765 |
| 13A6 | 7TATGCCGATAGCGTCAAG |
| CDR2 | |
| aVEGF- | ACTGCCGTCTATTATTGT66856587865787885787885 |
| 13A6 | 7558688888788757558776577857577678788657T |
| CDR3 | ACCGGGGTCAAGGAACA |

### Example 5: 13A6-Based Affinity Maturation Phage Display Selections

Neutravidin was immobilized onto Maxisorp Immuno Tubes (Thermo Scientific) at 10 μg in 1 mL PBS overnight at 4° C. The tubes were washed twice in PBS, and blocked for 1 h at room temperature (RT) in MBB. Biotinylated VEGF-121 (Acro Biosystems) was added at different concentrations depending on the panning round (refer to Table 4). The protein was incubated for 1 h at RT, and non-bound protein removed by two washes with PBS. Negative selection tubes were prepared as described for biotinylated VEGF-121, but PBS was added in place of biotinylated VEGF-121.

The remaining selection procedures are similar to the Phage display selections from naïve synthetic DotBody phage display libraries, with certain changes as summarised in Table 4.

TABLE 4
Affinity maturation selection conditions of 13A6-based
phage display libraries against biotinylated VEGF-121.

| Changes | Details |
|---|---|
| Number of | 5 rounds with increasing levels of stringency |
| selection rounds | |
| Adaptor protein | A biotin-binding protein was immobilized on Maxisorp plates as follows: |
| | Round 1: 20 μg Neutravidin in 2 mL |
| | Round 2: 10 μg Streptavidin in 2 mL |
| | Round 3: 10 μg Neutravidin in 2 mL |
| | Round 4: 10 μg Streptavidin in 2 mL |
| | Round 5: 10 μg Neutravidin in 2 mL |
| Amount of | Decreasing biotinylated VEGF-121 concentration was incubated on the |
| biotinylated VEGF- | adaptor protein-coated tubes in the various selection round: |
| 121 immobilized | Round 1 to 2: 300 nM |
| | Round 3 to 5: 15 nM |
| Amount of phages | The library was diluted in 1 mL MBB at different final concentrations in the |
| added to VEGF- | various selection round: |
| 121 | Round 1: 1 × 10¹³ pfu/mL of affinity matured phage display library |
| | Round 2: 1 × 10¹² pfu/mL of purified phages from previous round |
| | Rounds 3 to 5: 5 × 10¹¹ pfu/mL of purified phages from previous round |
| Incubation time of | The phages were incubated in the negative selection tube for 1 h at RT, then |
| phages with | transferred to the tube containing VEGF-121, and incubated at RT for |
| VEGF-121 | different durations in the various selection round: |
| | Round 1: 2 h |
| | Round 2: 1 h |
| | Round 3: 30 min |
| | Round 4: 10 min |
| | Round 5: 3 min |
| Washing of non- | Non-bound phages were removed following different wash regiments in the |
| bound phages | various selection round: |
| | Round 1: 3 times with MBB, 3 times with PBST, 2 times with PBS |
| | Round 2: 4 times with MBB, 4 times with PBST, 2 times with PBS |
| | Round 3: 10 times with MBB, 10 times with PBST, 2 times with PBS |
| | Rounds 4 and 5: 15 times with MBB, 15 times with PBST, 2 times with |
| | PBS |

13A6-based affinity maturation phage display selections gave rise to 16A2.1, 16C2.1 and 16A6.1.

### Example 6: 16A2.1-Based Affinity Maturation Phage Display Library Construction

Anti-VEGF DotBody 16A2.1 was selected for affinity maturation, as its binding affinity is below 5 nM and it also blocked the interaction between VEGFA and the VEGF receptor 1 (VEGFR1). An affinity maturation phage display library was created by Kunkel mutagenesis, according to Bostrom J. et al. (14), by using the primers stated in Table 5. As CDR3 contained a potential N-glycosylation site (sequence “NST”), three primers were designed for this CDR, one in which the parental sequence was left intact, and two others in which it was changed as follows before performing the randomized primer design (changes underlined):

16A2.1(AST):
SEVQLVESGGGLVQPGGSLRLSCAISGFALAETDIDWVRQAPGKGLEWVA
RIFSSGGNTDYADSVKGRFTISADTSKNTVYLQMNSLRAEDTAVYYCGRS
DSIAYNVILPASTYTARDYRGQGTLVTVSS
16A2.1(NSA):
SEVQLVESGGGLVQPGGSLRLSCAISGFALAETDIDWVRQAPGKGLEWVA
RIFSSGGNTDYADSVKGRFTISADTSKNTVYLQMNSLRAEDTAVYYCGRS
DSIAYNVILPNSAYTARDYRGQGTLVTVSS

The library obtained contained 0.6×10⁸ unique sequences, as estimated by serial dilutions upon library electroporation into TG1 cells, plating onto 2YT agar supplemented with 100 μg/mL Carbenicillin, and subsequent sequencing of plasmids from several colonies to determine mutation rates.

TABLE 5
DNA sequences of primers employed to generate
an affinity maturation phage display library
of anti-VEGF DotBody 16A2.1. The numbers in
the sequence represent frequency of bases A,
T, C and G in the oligonucleotide sequence.
Frequency code 5 = 70% A, 10% G, 10% C, 10%
T; 6 = 70% G, 10% A, 10% C, 10% T; 7 = 70% C,
10% A, 10% G, 10% T; 8 = 70% T, 10% A, 10% G,
10% C.

| Primer | |
|---|---|
| name | Primer sequence |
| aVEGF- | TGTGCAATTTCTGGCTTC678788678655577657ATAGA |
| 16A2.1 | CTGGGTGCGTCAG |
| CDR1 | |
| aVEGF- | GAATGGGTTGCAAGGATT88887887866866855857765 |
| 16A2.1 | 7TATGCCGATAGCGTCAAG |
| CDR2 | |
| aVEGF- | ACTGCCGTCTATTATTGT66856587865787858867885 |
| 16A2.1 | 7558688588788776558878577857577678565657T |
| CDR3 | ACCGGGGTCAAGGAACA |
| aVEGF- | ACTGCCGTCTATTATTGT66856587865787858867885 |
| 16A2.1 | 7558688588788776558878678857577678565657T |
| (AST) | ACCGGGGTCAAGGAACA |
| CDR3 | |
| aVEGF- | ACTGCCGTCTATTATTGT66856587865787858867885 |
| 16A2.1 | 7558688588788776678878577857577678565657T |
| (NSA) | ACCGGGGTCAAGGAACA |
| CDR3 | |

### Example 7: 16C2.1-Based Affinity Maturation Phage Display Library Construction

Anti-VEGF DotBody 16C2.1 was selected for affinity maturation, as its binding affinity is below 5 nM and it also blocked the interaction between VEGFA and the VEGF receptor 1 (VEGFR1). An affinity maturation phage display library was created by Kunkel mutagenesis, according to Bostrom J. et al. (14), by using the primers stated in Table 6. The library obtained contained 1.1×10⁸ unique sequences, as estimated by serial dilutions upon library electroporation into TG1 cells, plating onto 2YT agar supplemented with 100 μg/mL Carbenicillin, and subsequent sequencing of plasmids from several colonies to determine mutation rates:

TABLE 6
DNA sequences of primers employed to generate
an affinity maturation phage display library
of anti-VEGF DotBody 16C2.1. The numbers in
the sequence represent frequency of bases A,
T, C and G in the oligonucleotide sequence.
Frequency code 5 = 70% A, 10% G, 10% C, 10%
T; 6 = 70% G, 10% A, 10% C, 10% T; 7 = 70% C,
10% A, 10% G, 10% T; 8 = 70% T, 10% A, 10% G,
10% C.

| Primer | |
|---|---|
| name | Primer sequence |
| aVEGF- | TGTGCAATTTCTGGCTTC878788678678577657ATAGA |
| 16C2.1 | CTGGGTGCGTCAG |
| CDR1 | |
| aVEGF- | GAATGGGTTGCAAGGATT88887877687865788857765 |
| 16C2.1 | 7TATGCCGATAGCGTCAAG |
| CDR2 | |
| aVEGF- | ACTGCCGTCTATTATTGT66856577665757785767885 |
| 16C2.1 | 7558588888788657577776577857558678788757T |
| CDR3 | ACCGGGGTCAAGGAACA |

### Example 8: 16A2.1-Based and 16C2.1-Based Affinity Maturation Phage Display Selections, with Heat Challenge

16A2.1-based and 16C2.1-based phage display libraries were panned separately, following identical procedures.

Neutravidin was immobilized onto Maxisorp Immuno Tubes (Thermo Scientific) at 10 μg in 1 mL PBS overnight at 4° C. The tubes were washed twice in PBS, and blocked for 1 h at room temperature (RT) in MBB. Biotinylated VEGF-121 (Acro Biosystems) was added at different concentrations depending on the panning round (refer to Table 7). The protein was incubated for 1 h at RT, and non-bound protein removed by two washes with PBS. Negative selection tubes were prepared as described for biotinylated VEGF-121, but PBS was added in place of biotinylated VEGF-121.

The remaining selection procedures are similar to the Phage display selections from naïve synthetic DotBody phage display libraries, with certain changes as summarised in Table 7.

TABLE 7
Affinity maturation selection conditions of 16A2.1-based and 16C2.1-
based phage display libraries against biotinylated VEGF-121.

| Changes | Details |
|---|---|
| Number of | 4 rounds with increasing levels of stringency |
| selection rounds | |
| Adaptor protein | A biotin-binding protein was immobilized on Maxisorp plates as follows: |
| | Round 1: 20 μg Neutravidin in 2 mL |
| | Round 2: 10 μg Neutravidin in 2 mL |
| | Round 3: 10 μg Neutravidin in 2 mL |
| | Round 4: 10 μg Neutravidin in 2 mL |
| Amount of | Decreasing biotinylated VEGF-121 concentration was incubated on the |
| biotinylated VEGF- | adaptor protein-coated tubes in the various selection round: |
| 121 immobilized | Round 1: 75 nM |
| | Round 2: 24 nM |
| | Round 3: 7.5 nM |
| | Round 4: 7.5 nM |
| Amount of phages | The library was diluted in 1 mL MBB at different final concentrations in the |
| added to VEGF-121 | various selection round: |
| | Round 1: 2 × 10¹² pfu/mL of affinity matured phage display library |
| | Round 2: 5 × 10¹¹ pfu/mL of purified phages from previous round |
| | Rounds 3: 5 × 10¹¹ pfu/mL of purified phages from previous round |
| | Rounds 4: 5 × 10¹¹ pfu/mL of purified phages from previous round |
| Heat challenge | The phages were incubated at different temperatures, spun-down at 15,000g |
| | for 5 min, before proceeding with selection. The temperatures are shown |
| | below: |
| | Round 1: no heat treatment |
| | Round 2: no heat treatment |
| | Round 3: 30 min at 55° C. |
| | Round 4: 30 min at 70° C. |
| Incubation time | The phages were incubated in the negative selection tube for 1 h at RT, then |
| of phages with | transferred to the tube containing VEGF-121, and incubated at RT for |
| VEGF-121 | different durations in the various selection round: |
| | Round 1: 1 h |
| | Round 2: 30 min |
| | Round 3: 30 min |
| | Round 4: 30 min |
| Washing of non- | Non-bound phages were removed following different wash regiments in the |
| bound phages | various selection round: |
| | Round 1: 3 times with MBB, 3 times with PBST |
| | Round 2: 10 times with MBB, 10 times with PBST, 2 times with PBS |
| | Round 3: 15 times with MBB, 15 times with PBST, 2 times with PBS |
| | Round 4: 15 times with MBB, 15 times with PBST, 2 times with PBS |

16A2.1-based affinity maturation phage display selections with heat challenge gave rise to 20A2.1 and 20A3.1.

16C2.1-based affinity maturation phage display selections with heat challenge gave rise to 21A1.1, 21A8.1, 21D9.1 and 21E6.1.

### Example 9: 16C2.1-Based Affinity Maturation Phage Display Selections, without Heat Challenge

The 1602.1-based phage display library was also panned without heat challenge, as follows.

Neutravidin was immobilized onto Maxisorp Immuno Tubes (Thermo Scientific) at 10 μg in 1 mL PBS overnight at 4° C. The tubes were washed twice in PBS, and blocked for 1 h at room temperature (RT) in MBB. Biotinylated VEGF-165 (Acro Biosystems) was added at different concentrations depending on the panning round (refer to Table 8). The protein was incubated for 1 h at RT, and non-bound protein removed by two washes with PBS. Negative selection tubes were prepared as described for biotinylated VEGF-165, but PBS was added in place of biotinylated VEGF-165.

The remaining selection procedures are similar to the Phage display selections from naïve synthetic DotBody phage display libraries, with certain changes as summarised in Table 8.

TABLE 8
Affinity maturation selection conditions of 16C2.1-based
phage display libraries against biotinylated VEGF-165.

| Changes | Details |
|---|---|
| Number of | 4 rounds with increasing levels of stringency |
| selection rounds | |
| Adaptor protein | A biotin-binding protein was immobilized on Maxisorp plates as follows: |
| | Round 1: 20 μg Neutravidin in 2 mL |
| | Round 2: 10 μg Streptavidin in 2 mL |
| | Round 3: 10 μg Neutravidin in 2 mL |
| | Round 4: 10 μg Neutravidin in 2 mL |
| Amount of | Decreasing biotinylated VEGF-165 concentration was incubated on the |
| biotinylated VEGF- | adaptor protein-coated tubes in the various selection round: |
| 165 immobilized | Round 1: 223 nM |
| | Round 2: 223 nM |
| | Round 3: 11 nM |
| | Round 4: 11 nM |
| Amount of phages | The library was diluted in 1 mL MBB at different final concentrations in the |
| added to VEGF- | various selection round: |
| 165 | Round 1: 1 × 10¹³ pfu/mL of affinity matured phage display library |
| | Round 2: 1 × 10¹² pfu/mL of purified phages from previous round |
| | Rounds 3: 1 × 10¹² pfu/mL of purified phages from previous round |
| | Rounds 4: 5 × 10¹¹ pfu/mL of purified phages from previous round |
| Incubation | The phages were incubated in the negative selection tube for 1 h at RT, then |
| conditions of | transferred to the tube containing VEGF-165, and incubated at RT for |
| phages with | different durations in the various selection round, with ranibizumab added for |
| VEGF-165 | additional stringency in rounds 3 and 4: |
| | Round 1: 1 h |
| | Round 2: 30 min |
| | Round 3: 30 min, in the presence of 5 nM ranibizumab |
| | Round 4: 30 min, in the presence of 5 nM ranibizumab |
| Washing of non- | Non-bound phages were removed following different wash regiments in the |
| bound phages | various selection round: |
| | Round 1: 3 times with MBB, 3 times with PBST |
| | Round 2: 10 times with MBB, 10 times with PBST, 2 times with PBS |
| | Round 3: 15 times with MBB, 15 times with PBST, 2 times with PBS |
| | Round 4: 15 times with MBB, 15 times with PBST, 2 times with PBS |

### Example 10: Protein Production and Characterization of Binding Kinetics by Biolayer Interferometry

VEGFA-binding clones were cloned into a pET-based expression vector with a ATG codon in 5′ of the open reading frame and a sequence coding for a hexahistidine tag in 3′. They were produced recombinantly in E. coli and purified by immobilized-metal affinity chromatography, followed by desalting into PBS. The bivalent molecule 16A2.1x2 was also produced in the same way.

Binding characterization was performed using BLI (Satorius) at RT with a 1000 rpm flow-rate. Biotinylated human VEGF-165 (Acro Biosystem) was immobilized onto Streptavidin-coated tips at a concentration of 3 μg/mL for 60 sec in BLI buffer (0.1% BSA and 0.01% Tween-20 in PBS). After a 30 sec baseline, anti-VEGFA DotBodies were associated at 8 different concentrations, including a blank reference, in BLI buffer for 60 sec, followed by a 400 sec dissociation in BLI buffer. The background buffer signal was subtracted using the 0 nM concentration reference, and the kinetics of binding were calculated using a global fit following a 1:1 binding model, using BLI Analysis Software. The bivalent molecule 16A2.1x2 was also characterized as described here, but with a 600 sec dissociation. To characterize the binding of all the clones against murine VEGFA, the same procedure was employed, but the immobilized target was replaced with biotinylated murine VEGF-164 (Acro Biosystem).

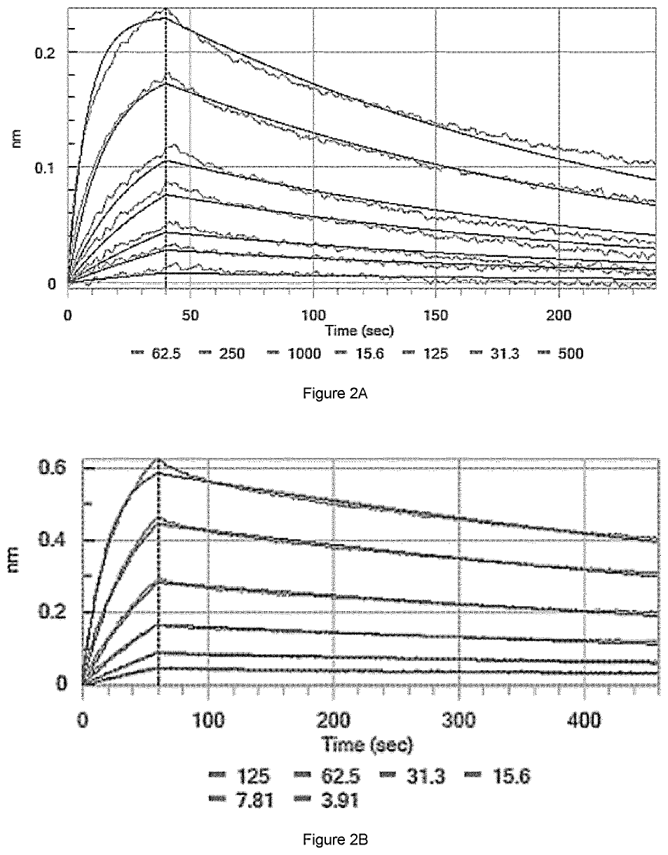

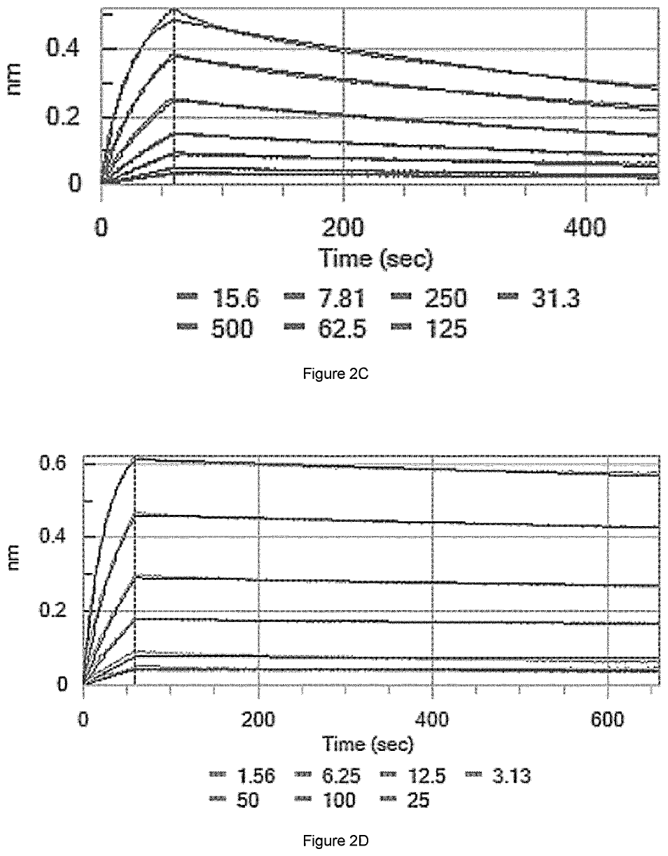

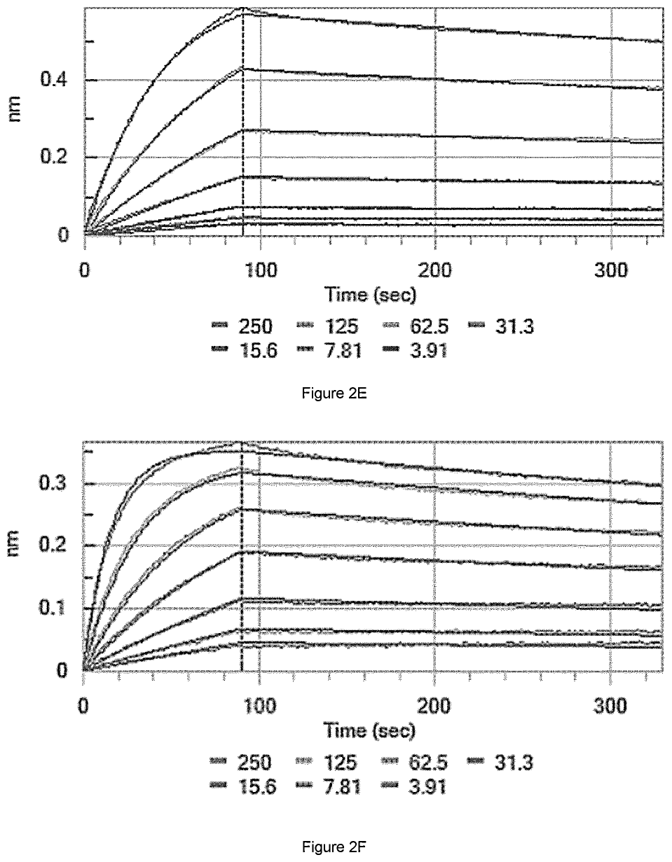

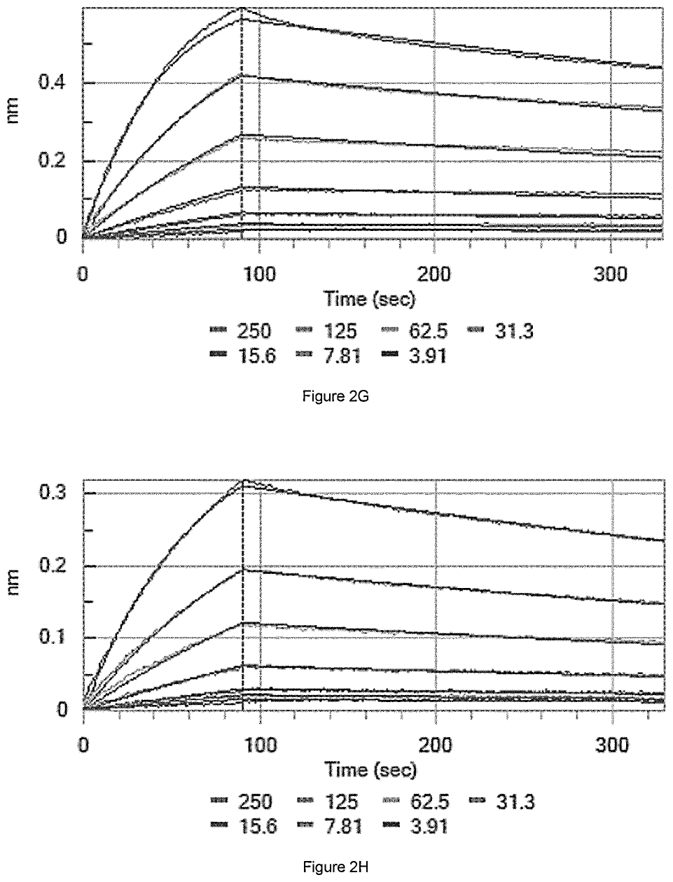

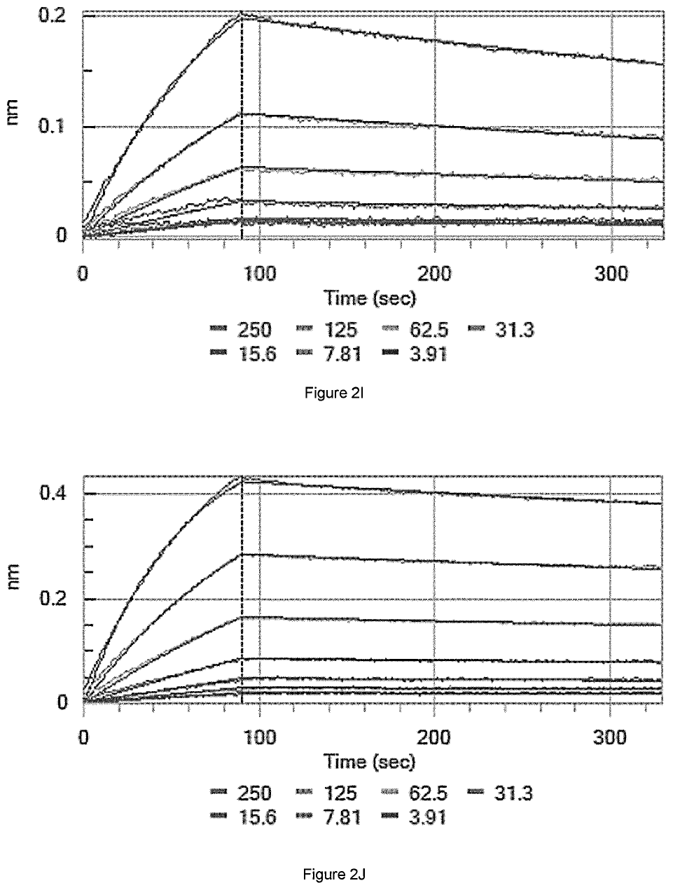

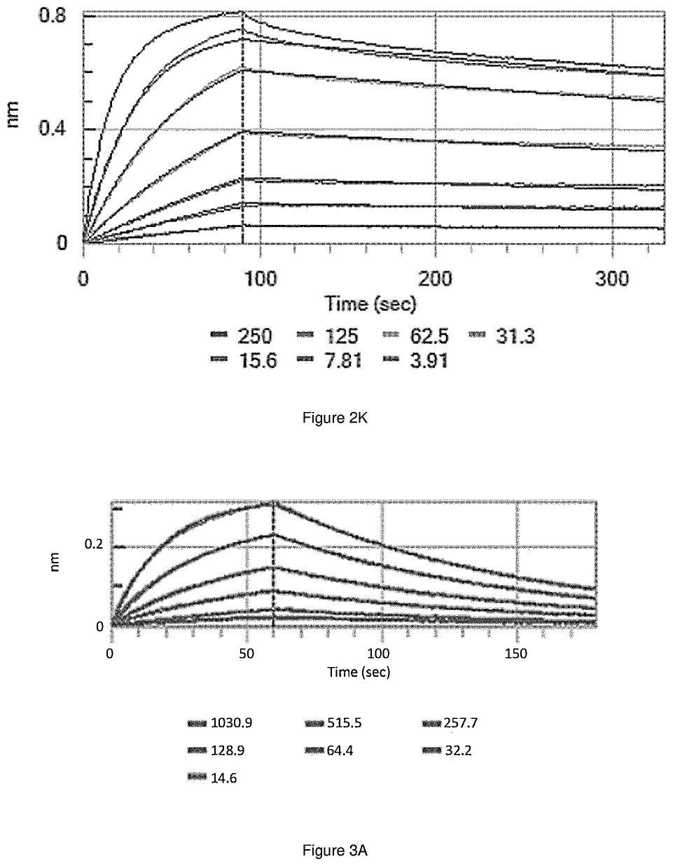

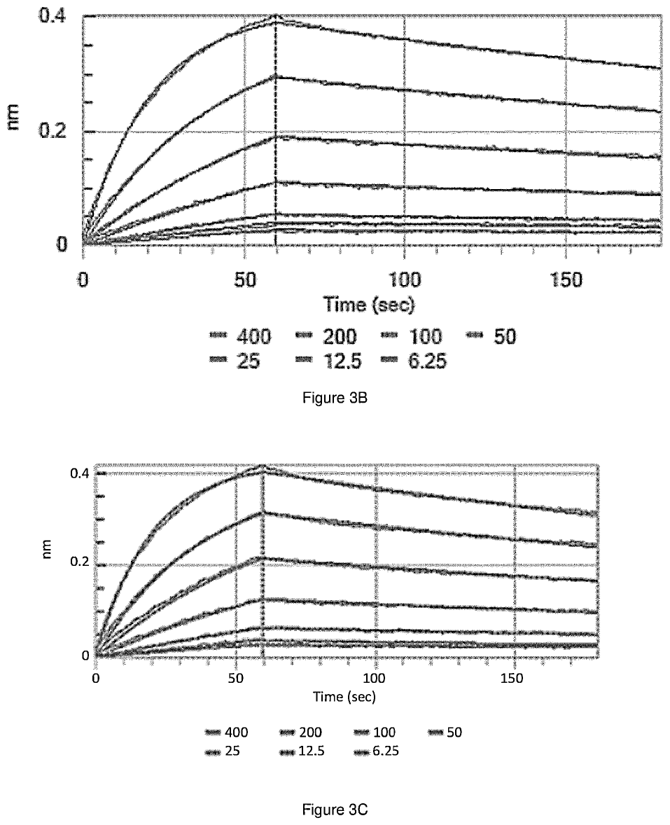

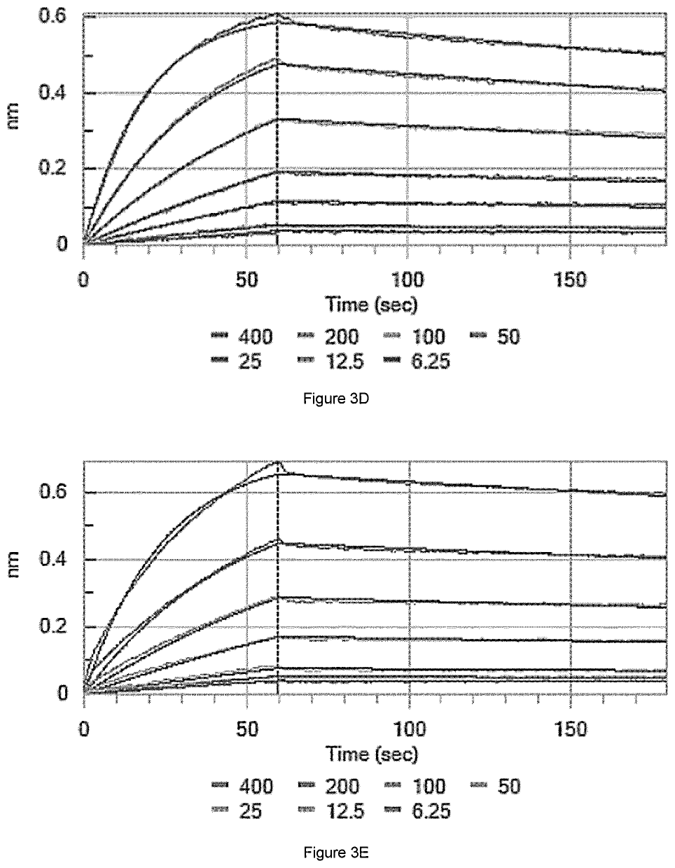

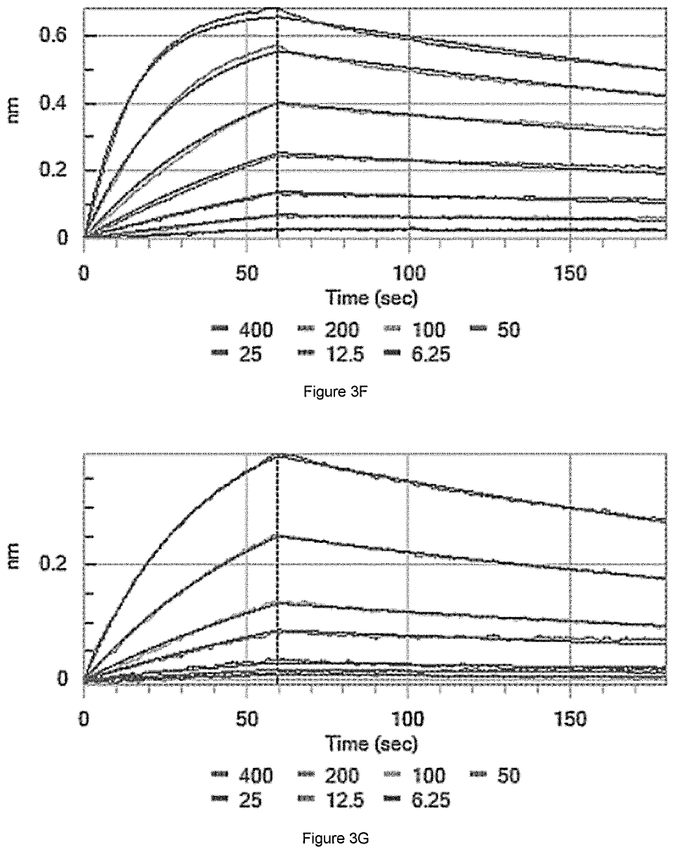

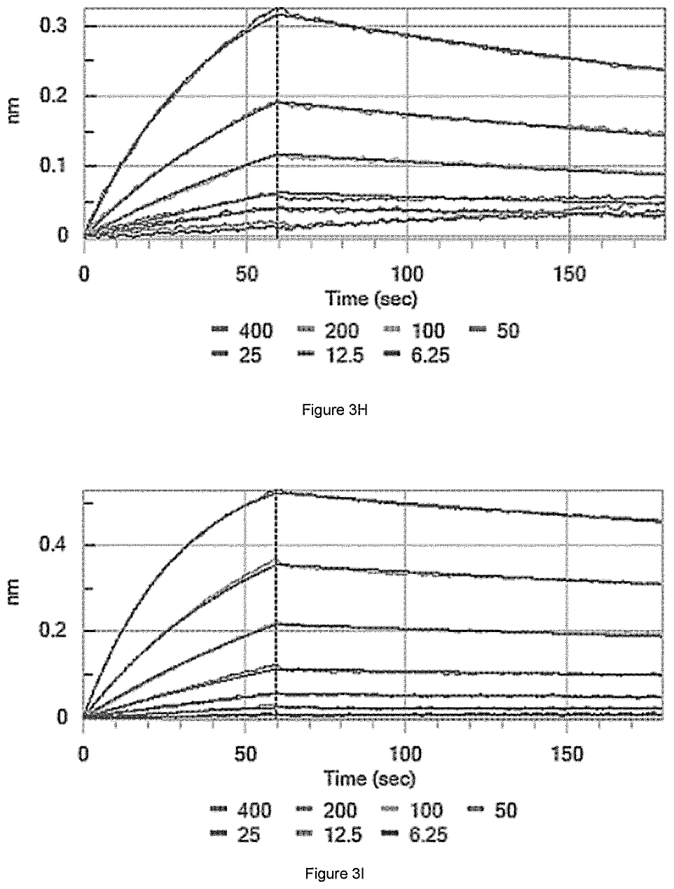

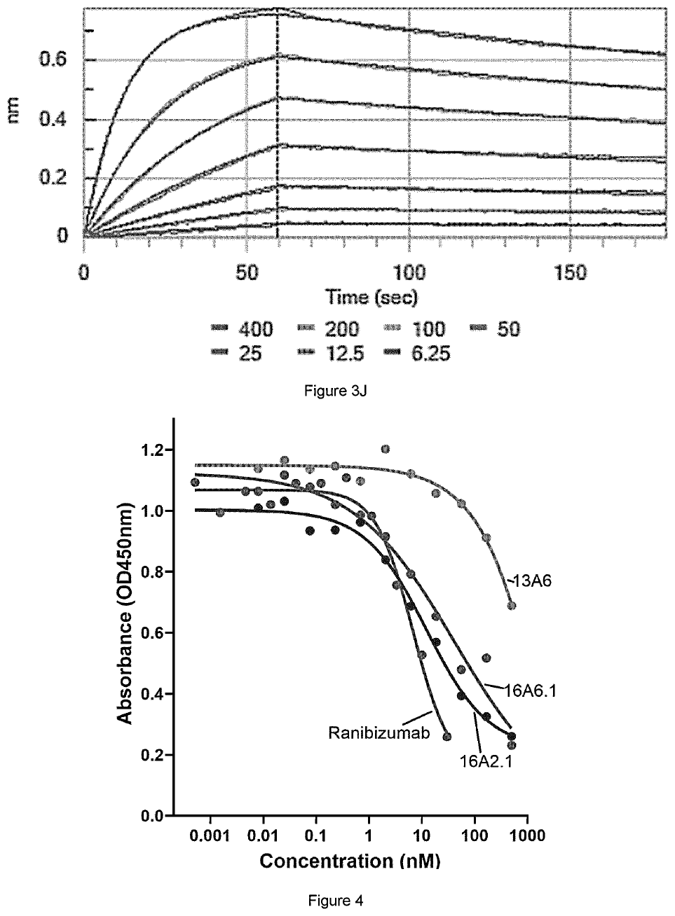

The sensorgrams are shown in FIGS. 2 and 3, and the binding data are shown in the following table:

| Target | | Kₒₙ | K_{diss} | Binding Affinity | |
|---|---|---|---|---|---|
| antigen | Molecule | (M⁻¹s⁻¹) | (s⁻¹) | (K_{D}) | |
| Human | 13A6 | 1.22 × 10⁵ | 4.75 × 10³ | 38.9 | nM |
| VEGF165 | 16A2.1 | 3.94 × 10⁵ | 9.86 × 10⁻⁴ | 2.5 | nM |
| | 16A6.1 | 9.24 × 10⁴ | 1.36 × 10⁻³ | 14.7 | nM |
| | 16A2.1x2 | 4.26 × 10⁵ | 1.28 × 10⁻⁴ | 0.301 | nM |
| | 20A2.1 | 9.77 × 10⁴ | 5.50 × 10⁻⁴ | 5.54 | nM |
| | 20A3.1 | 2.60 × 10⁵ | 7.25 × 10⁻⁴ | 2.78 | nM |
| | 21A1.1 | 9.15 × 10⁴ | 1.03 × 10⁻³ | 11.3 | nM |
| | 21A8.1 | 5.80 × 10⁴ | 1.18 × 10⁻³ | 20.0 | nM |
| | 21D9.1 | 4.82 × 10⁴ | 9.90 × 10⁻⁴ | 20.5 | nM |
| | 21E6.1 | 6.96 × 10⁴ | 4.42 × 10⁻⁴ | 6.3 | nM |
| | 23D5.1 | 2.86 × 10⁵ | 8.02 × 10⁻⁴ | 2.8 | nM |
| Mouse | 13A6 | 4.19 × 10⁴ | 1.04 × 10⁻² | 248 | nM |
| VEGF164 | 16A2.1 | 1.16 × 10⁵ | 1.93 × 10⁻³ | 16.6 | nM |
| | 16A6.1 | 1.22 × 10⁵ | 2.25 × 10⁻³ | 18.4 | nM |
| | 20A2.1 | 1.32 × 10⁵ | 1.35 × 10⁻³ | 10.2 | nM |
| | 20A3.1 | 1.02 × 10⁵ | 8.89 × 10⁻⁴ | 8.7 | nM |
| | 21A1.1 | 1.67 × 10⁵ | 2.31 × 10⁻³ | 13.8 | nM |
| | 21A8.1 | 6.34 × 10⁴ | 2.92 × 10⁻³ | 46.1 | nM |
| | 21D9.1 | 5.80 × 10⁴ | 2.43 × 10⁻³ | 41.7 | nM |
| | 21E6.1 | 8.28 × 10⁴ | 1.17 × 10⁻³ | 14.2 | nM |
| | 23D5.1 | 2.05 × 10⁵ | 1.69 × 10⁻³ | 8.34 | nM |

### Example 11: Competitive ELISA

To perform the competitive ELISA, the VEGFR1 was immobilized at a concentration of 2 μg/mL onto a Maxisorp 96-well plate (Thermo Scientific) overnight at 4° C., washed three times with PBS and blocked for 1 h at RT with ELISA Block Buffer (EBB: 0.2% BSA in PBST). Human VEGF-121 at a concentration 0.5 nM was mixed with purified anti-VEGFA DotBodies at different concentrations following a 1:3 serial dilution starting at 500 nM and ending at 0.008 nM, with a 0 nM control. Ranibizumab was also mixed with human VEGF-121 using a 1:3 serial dilution starting at 30 nM and ending at 0.0005 nM, with a 0 nM control. The samples were incubated for 2 h at RT, and 50 μL transferred onto the VEGFR1-coated plate. After 2 h incubation, the plate was washed 3 times with PBST. 50 μL of streptavidin-HRP conjugate (Thermo Scientific) was added at a 1:5,000 dilution in EBB, and incubated for 1 h at RT. The plate was washed 5 times with PBST, and developed with 50 μL 3,3′,5,5′-Tetramethylbenzidine (TMB) substrate (GeneTex). After 5-15 min, the reactions were stopped by adding 50 μL of 2M H₂SO₄, and signal was measured at an absorbance of 450 nm.

The data was plotted using Graphpad Prism 9 software, and IC₅₀ determined using a “[inhibitor] vs. response—variable slope (four parameters)” non-linear regression curve.

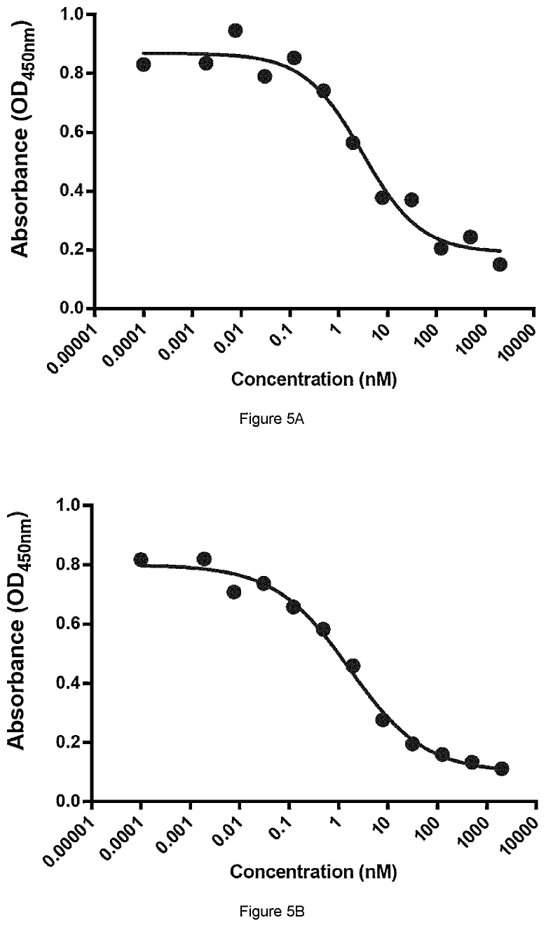

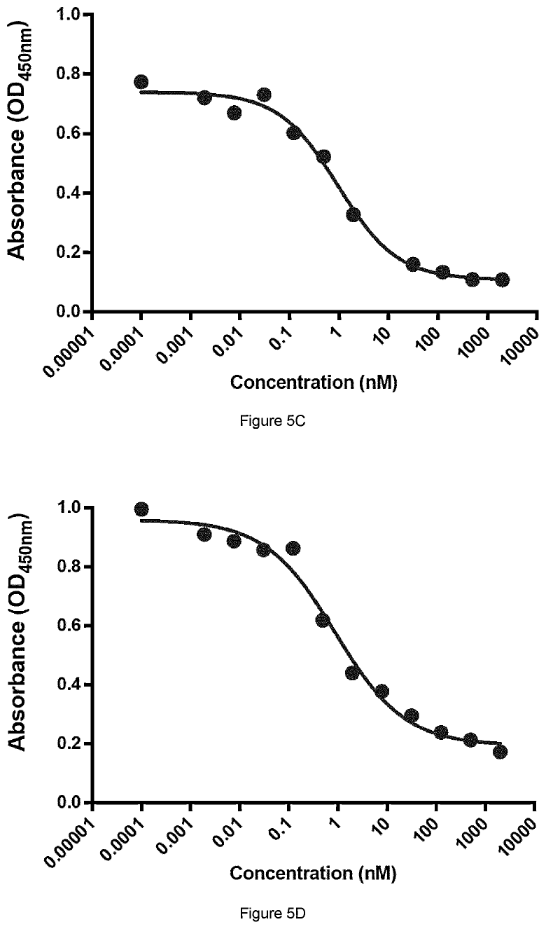

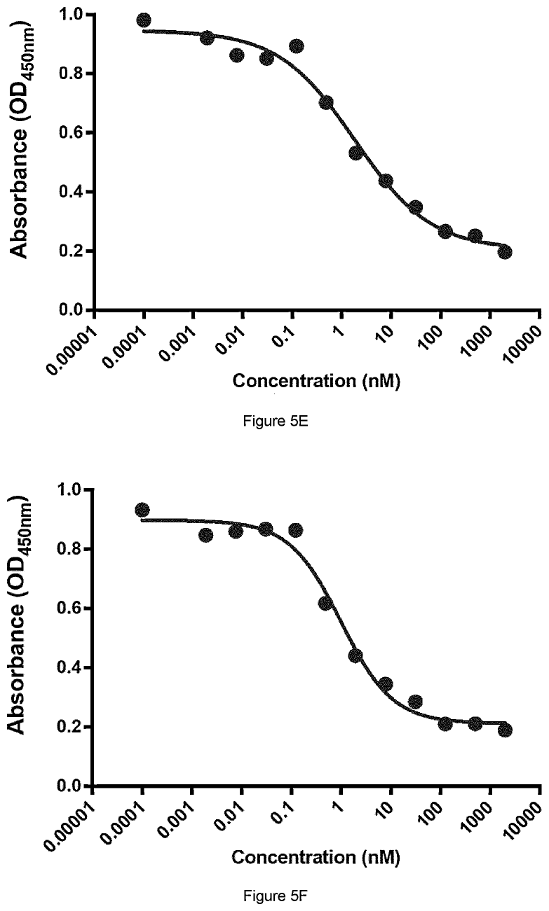

The results are shown in FIGS. 4 and 5.

The IC₅₀ values determined for the different molecules for inhibition of interaction between human VEGF-121 and human VEGFR1 based on the data in FIG. 4 were as follows:
13A6=5.9 μM
16A2.1=12.2 nM
16A6.1=41.4 nM
Ranibizumab=6.8 nM.

The IC₅₀ values determined for the different molecules for inhibition of interaction between human VEGF-121 and human VEGFR1 based on the data in FIG. 5 were as follows:
16A2.1=3.0 nM
20A2.1=1.6 nM
20A3.1=0.9 nM
21A1.1=0.9 nM
21A8.1=1.8 nM
21D9.1=1.0 nM
21E6.1=7.5 nM
23D5.1=1.4 nM
Ranibizumab=7.4 nM

### Example 12: Evaluation of Thermostability by Analysis of Binding to VEGFA after Heat Challenge

VEGFA-binding DotBodies were incubated for 1 h at room temperature, 60° C., 70° C. or 80° C. in BLI buffer, at a concentration of 250 nM (heat challenge). After the heat challenge, the samples were centrifuged at 15,000 g for 5 min and the supernatant was employed to perform characterization of binding to human VEGF165 by BLI, as described in Example 10 above.

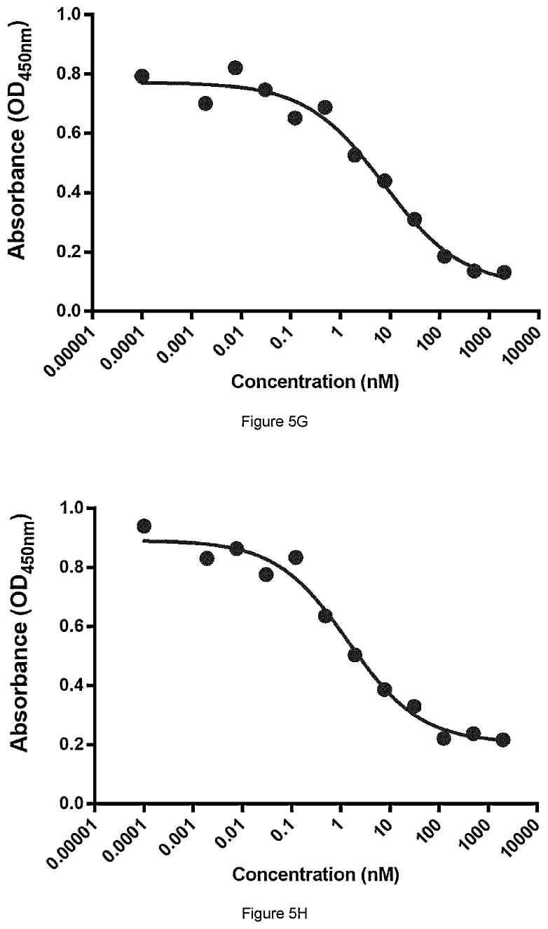

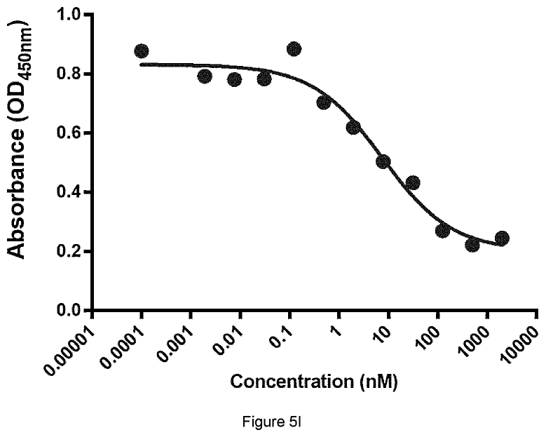

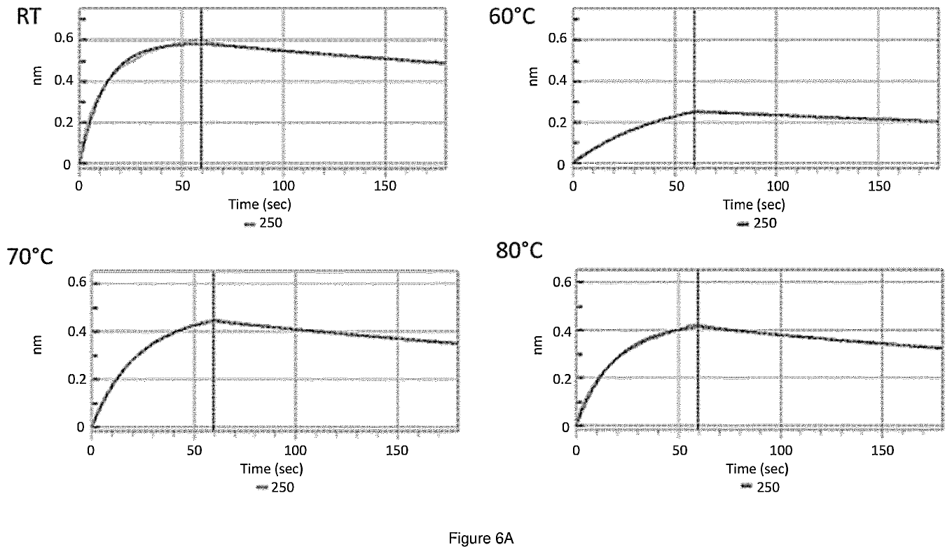

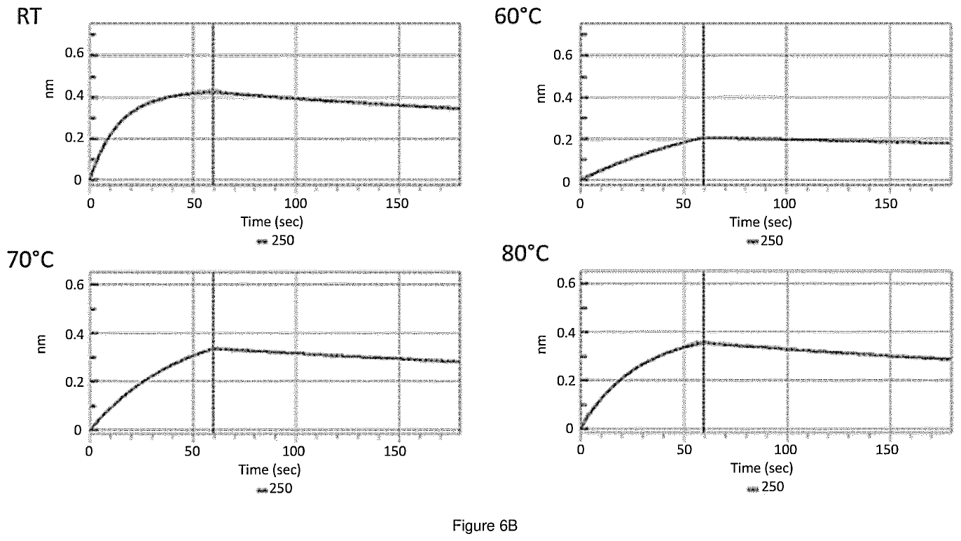

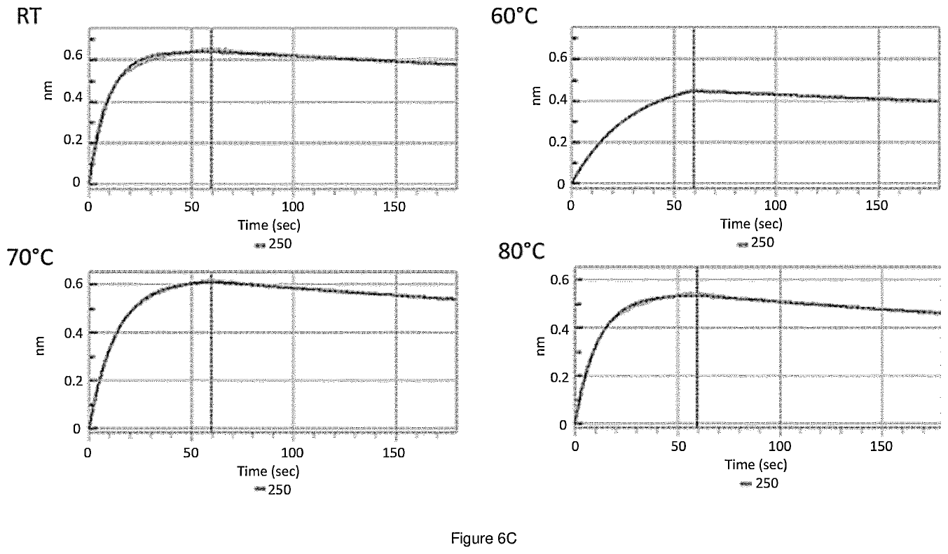

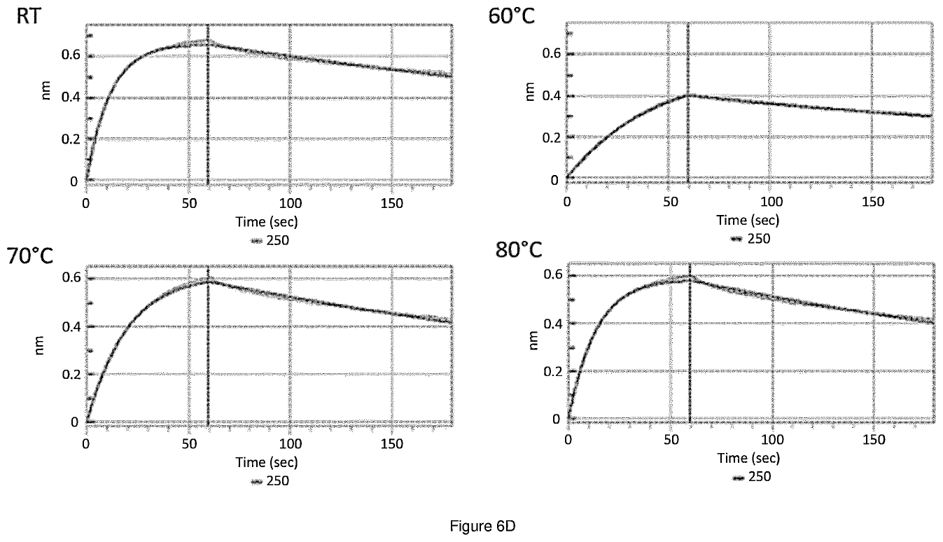

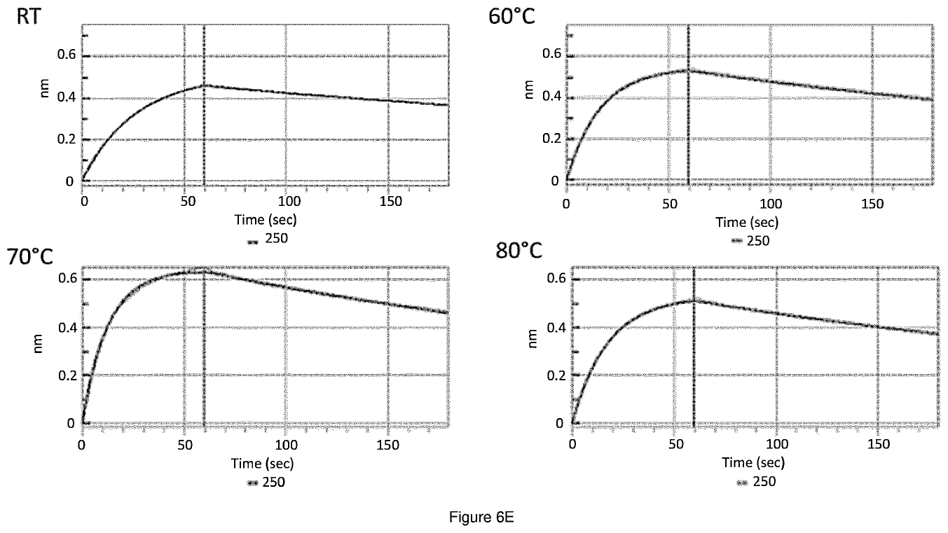

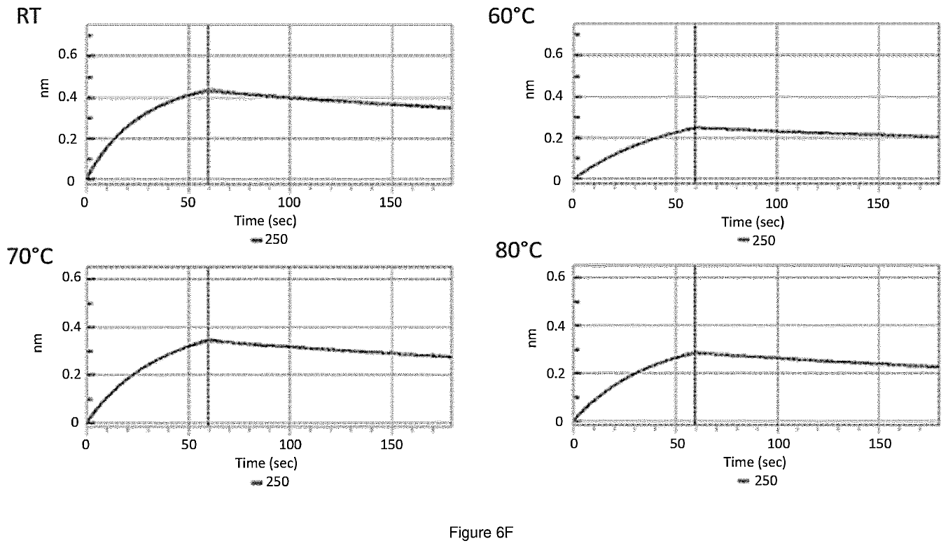

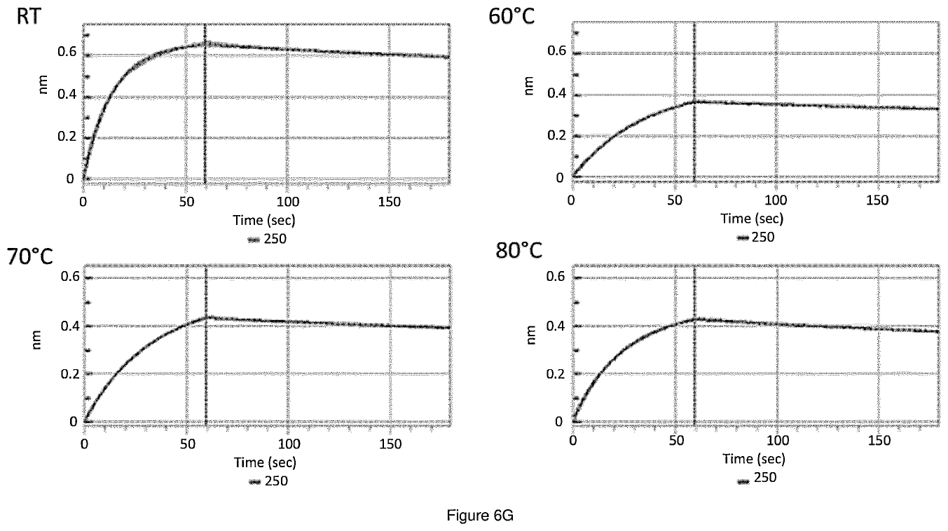

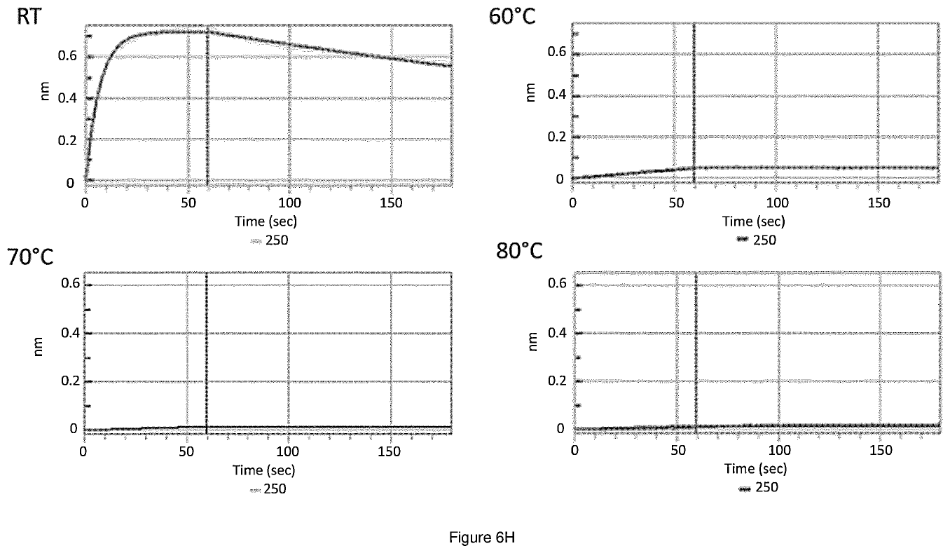

The results are shown in FIG. 6.

### Example 13: Conclusions

The inventors have produced stabilized VH domain antibodies, which bind specifically to human VEGFA, and which cross-react with murine VEGFA.

Among the antigen-binding molecules produce, clone 13A6 has an affinity of 38.9 nM for human VEGFA and 248 nM for murine VEGFA. Clone 13A6 blocks the interaction between VEGFA and VEGF Receptor 1 (VEGFR1) with an IC₅₀ of 5.9 μM as measured by competitive ELISA.

13A6 was subjected to affinity maturation by phage display to further improve its binding affinity against human and murine VEGFA. A new phage display library was generated, in which each CDR position was mutated with a ratio of approximately 50% of the residue present in 13A6's wild-type sequence, and 50% of any other amino acid. Affinity maturation selections against human VEGFA were performed with increasing levels of stringency, by lowering phage concentration, antigen concentration and binding time, while increasing the number of washes. This procedure led to the identification of several affinity-improved DotBodies.

Among these, clones, 16A2.1 and 16A6.1 have affinities against human VEGFA of 2.5 nM and 14.7 nM, respectively. Their affinities for murine VEGFA are 16.6 nM and 18.4 nM, respectively. Their abilities to block the VEGFA-VEGFR1 interaction were significantly improved, with IC50s estimated at 12.2 nM for 16A2.1 and 41.4 nM for 16A6.1.

To prove the modularity of the DotBodies generated against VEGFA, clone 16A2.1 was produced as a bivalent molecule comprising of two copies of 16A2.1 sequence connected by a flexible “(GGGGS)x6-GGGG” linker. The molecule could be produced recombinantly in E. coli, and had an improved affinity for VEGFA of 301 μM. As VEGFA exists as a homodimer (11), the improved binding affinity suggests that the bivalent 16A2.1x2 molecule may be binding both VEGF monomers concurrently.

DotBodies 16A2.1 and 16C2.1 were selected for further activity improvements. Additionally, 16A2.1 contained a potential glycosylation site (“NST”) in CDR3, which needed to be removed while maintaining binding. Two new phage display libraries were generated, in which each CDR position was mutated with a ratio of approximately 50% of the residue present in 16A2.1 and 16C2.1 sequences, respectively, and 50% of any other amino-acid. For 16A2.1, the “NST” sequence in CDR3 was replaced by either “AST” or “NSA” when performing the affinity maturation library design, with the goal of removing either Asn (N) or Ser (S)/Thr (T) in the typical Nx(T/S)N-glycosylation motif (where “x” is any amino acid, except proline) (12,13). Binding and stability-based selections against human VEGFA were performed with heat challenges at increasing temperatures, while lowering the antigen concentration and increasing the number of washes, to identify the most stable, high affinity anti-VEGF DotBodies. An additional selection without heat challenge was performed for the 16C2.1-based library. The heat-challenged libraries led to the identification of clones 20A2.1, 20A3.1, 21A1.1, 21A8.1, 21D9.1, 21E9.1, while the non-heat-challenged selection identified DotBody 23D5.1 as the most enriched sequence. Importantly, DotBodies 20A2.1 and 20A3.1 did not contain the potential N-glycosylation site present in their parental clone 16A2.1.

All DotBodies identified retained binding to human VEGFA with affinities ranging from 2.8 nM to 20 nM, and to mouse VEGFA with affinities ranging from 8.3 nM to 46.1 nM. Their ability to block VEGF-VEGFR interaction was measured by competitive ELISA, with IC50s ranging from 0.9 nM to 7.5 nM. Ranibizumab was used as a control, with a measured IC50 of 7.4 nM. Parental DotBody 16A2.1 had an IC50=3 nM, and the newly identified DotBodies 20A2.1 and 20A3.1 showed improved blockade activity (IC50=1.6 nM and IC50=0.9 nM, respectively). The following DotBodies also displayed high blockade activity: 21A1.1 (IC50=0.9 nM), 21D9.1 (IC50=1.0 nM) and 23D5.1 (IC50=1.4 nM).

All the DotBodies identified through the heat-challenge selection retained binding to VEGFA after incubation at temperatures ranging between room-temperature and 80° C. 23D5.1, which was identified in selections without heat challenge, lost most of its binding activity at 60° C. or above.

In conclusion, through a series of tailored phage display library designs and selection strategies, the inventors produced a broad range of anti-VEGF DotBodies with mid- to low-nanomolar affinities, binding both human VEGFA and murine VEGFA. These DotBodies are able to block the VEGF-VEGFR interactions with IC50s in the low-micromolar to sub-nanomolar range. The majority of DotBodies identified, including five potent blockers—16A2.1, 20A2.1, 20A3.1 and 21D9.1—have high thermostability, retaining binding to human VEGFA after incubation at temperatures ranging from room temperature to 80° C. These DotBodies are modular, and can used to create multi-valent and/or multi-specific molecules by linking several DotBodies in tandem (e.g. by introducing linker sequences between the DotBodies).

### REFERENCES

1. Fogli, S., Del Re, M., Rofi, E., Posarelli, C., Figus, M., and Danesi, R. (2018) Clinical pharmacology of intravitreal anti-VEGF drugs. Eye (Lond) 32, 1010-1020
2. Osaadon, P., Fagan, X. J., Lifshitz, T., and Levy, J. (2014) A review of anti-VEGF agents for proliferative diabetic retinopathy. Eye (Lond) 28, 510-520
3. Meadows, K. L., and Hurwitz, H. I. (2012) Anti-VEGF therapies in the clinic. Cold Spring Harb Perspect Med 2
4. Williams, K. A., Brereton, H. M., Farrall, A., Standfield, S. D., Taylor, S. D., Kirk, L. A., and Coster, D. J. (2005) Topically applied antibody fragments penetrate into the back of the rabbit eye. Eye (Lond) 19, 910-913
5. Thiel, M. A., Coster, D. J., Standfield, S. D., Brereton, H. M., Mavrangelos, C., Zola, H., Taylor, S., Yusim, A., and Williams, K. A. (2002) Penetration of engineered antibody fragments into the eye. Clin Exp Immunol 128, 67-74
6. Kontermann, R. E. (2012) Dual targeting strategies with bispecific antibodies. mAbs 4, 182-197
7. Brinkmann, U., and Kontermann, R. E. (2017) The making of bispecific antibodies. mAbs 9, 182-212
8. Deshaies, R. J. (2020) Multispecific drugs herald a new era of biopharmaceutical innovation. Nature 580, 329-338
9. Cornvik, T., Dahlroth, S. L., Magnusdottir, A., Herman, M. D., Knaust, R., Ekberg, M., and Nordlund, P. (2005) Colony filtration blot: a new screening method for soluble protein expression in Escherichia coli. Nat Methods 2, 507-509
10. Asial, I., Cheng, Y. X., Engman, H., Dollhopf, M., Wu, B., Nordlund, P., and Cornvik, T. (2013) Engineering protein thermostability using a generic activity-independent biophysical screen inside the cell. Nature communications 4, 2901
11. Muller, Y. A., Li, B., Christinger, H. W., Wells, J. A., Cunningham, B. C., and de Vos, A. M. (1997) Vascular endothelial growth factor: crystal structure and functional mapping of the kinase domain receptor binding site. Proc Natl Acad Sci USA 94, 7192-7197
12. Bause, E. (1983) Structural requirements of N-glycosylation of proteins. Studies with proline peptides as conformational probes. Biochem J 209, 331-336
13. Bause, E., and Hettkamp, H. (1979) Primary structural requirements for N-glycosylation of peptides in rat liver. FEBS Lett 108, 341-344
14. Bostrom, J., Lee, C. V., Haber, L., and Fuh, G. (2009) Improving antibody binding affinity and specificity for therapeutic development. Methods Mol Biol 525, 353-376, xiii
15. Bostrom, J., and Fuh, G. (2009) Design and construction of synthetic phage-displayed Fab libraries. Methods Mol Biol 562, 17-35
16. Tonikian, R., Zhang, Y., Boone, C., and Sidhu, S. S. (2007) Identifying specificity profiles for peptide recognition modules from phage-displayed peptide libraries. Nature protocols 2, 1368-1386

1. An antigen-binding molecule, optionally isolated, which binds to VEGFA, wherein the antigen-binding molecule comprises a single domain antibody sequence incorporating the following CDRs:
CDR1 having the amino acid sequence of SEQ ID NO:45
CDR2 having the amino acid sequence of SEQ ID NO:46
CDR3 having the amino acid sequence of SEQ ID NO:48.

2. The antigen-binding molecule according to claim 1, wherein the antigen-binding molecule comprises, or consists of, an amino acid sequence having at least 70% sequence identity to the amino acid sequence of SEQ ID NO:49.

3. The antigen-binding molecule according to claim 1 or claim 2, wherein the antigen-binding molecule comprises a single domain antibody sequence incorporating the following FRs:
FR1 having the amino acid sequence of SEQ ID NO:40 FR2 having the amino acid sequence of SEQ ID NO:41 FR3 having the amino acid sequence of SEQ ID NO:47 FR4 having the amino acid sequence of SEQ ID NO:44.

4. The antigen-binding molecule according to any one of claims 1 to 3, wherein the antigen-binding molecule comprises a single domain antibody sequence incorporating the following CDRs:
CDR1 having the amino acid sequence of SEQ ID NO:50
CDR2 having the amino acid sequence of SEQ ID NO:51
CDR3 having the amino acid sequence of SEQ ID NO:52.

5. The antigen-binding molecule according to any one of claims 1 to 4, wherein the antigen-binding molecule comprises, or consists of, an amino acid sequence having at least 70% sequence identity to the amino acid sequence of SEQ ID NO:53.

6. The antigen-binding molecule according to any one of claims 1 to 5, wherein the antigen-binding molecule comprises:
(i) a single domain antibody sequence incorporating the following CDRs:
CDR1 having the amino acid sequence of SEQ ID NO:17
CDR2 having the amino acid sequence of SEQ ID NO:18
CDR3 having the amino acid sequence of SEQ ID NO:19; or
(ii) a single domain antibody sequence incorporating the following CDRs:
CDR1 having the amino acid sequence of SEQ ID NO:6
CDR2 having the amino acid sequence of SEQ ID NO:7
CDR3 having the amino acid sequence of SEQ ID NO:8; or
(iii) a single domain antibody sequence incorporating the following CDRs:
CDR1 having the amino acid sequence of SEQ ID NO:13
CDR2 having the amino acid sequence of SEQ ID NO:14
CDR3 having the amino acid sequence of SEQ ID NO:15.

7. The antigen-binding molecule according to any one of claims 1 to 6, wherein the antigen-binding molecule comprises, or consists of, an amino acid sequence having at least 70% sequence identity to the amino acid sequence of SEQ ID NO:16, 5 or 12.

8. The antigen-binding molecule according to any one of claims 1 to 3, wherein the antigen-binding molecule comprises a single domain antibody sequence incorporating the following CDRs:
CDR1 having the amino acid sequence of SEQ ID NO:54
CDR2 having the amino acid sequence of SEQ ID NO:55
CDR3 having the amino acid sequence of SEQ ID NO:56.

9. The antigen-binding molecule according to any one of claims 1 to 3 or claim 8, wherein the antigen-binding molecule comprises, or consists of, an amino acid sequence having at least 70% sequence identity to the amino acid sequence of SEQ ID NO:57.

10. The antigen-binding molecule according to any one of claims 1 to 3, claim 8 or claim 9, wherein the antigen-binding molecule comprises:
(i) a single domain antibody sequence incorporating the following CDRs:
CDR1 having the amino acid sequence of SEQ ID NO:21
CDR2 having the amino acid sequence of SEQ ID NO:22
CDR3 having the amino acid sequence of SEQ ID NO:23; or
(ii) a single domain antibody sequence incorporating the following CDRs:
CDR1 having the amino acid sequence of SEQ ID NO:25
CDR2 having the amino acid sequence of SEQ ID NO:26
CDR3 having the amino acid sequence of SEQ ID NO:27; or
(iii) a single domain antibody sequence incorporating the following CDRs:
CDR1 having the amino acid sequence of SEQ ID NO:29
CDR2 having the amino acid sequence of SEQ ID NO:30
CDR3 having the amino acid sequence of SEQ ID NO:31; or
(iv) a single domain antibody sequence incorporating the following CDRs:
CDR1 having the amino acid sequence of SEQ ID NO:33
CDR2 having the amino acid sequence of SEQ ID NO:34
CDR3 having the amino acid sequence of SEQ ID NO:35; or
(v) a single domain antibody sequence incorporating the following CDRs:
CDR1 having the amino acid sequence of SEQ ID NO:37
CDR2 having the amino acid sequence of SEQ ID NO:38
CDR3 having the amino acid sequence of SEQ ID NO:39.

11. The antigen-binding molecule according to any one of claims 1 to 3 or claims 8 to 10, wherein the antigen-binding molecule comprises, or consists of, an amino acid sequence having at least 70% sequence identity to the amino acid sequence of SEQ ID NO:20, 24, 28, 32 or 36.

12. The antigen-binding molecule according to any one of claims 1 to 3, wherein the antigen-binding molecule comprises a single domain antibody sequence incorporating the following CDRs:
CDR1 having the amino acid sequence of SEQ ID NO:2
CDR2 having the amino acid sequence of SEQ ID NO:3
CDR3 having the amino acid sequence of SEQ ID NO:4.

13. The antigen-binding molecule according to any one of claims 1 to 3 or claim 12, wherein the antigen-binding molecule comprises, or consists of, an amino acid sequence having at least 70% sequence identity to the amino acid sequence of SEQ ID NO:1.

14. The antigen-binding molecule according to any one of claims 1 to 3, wherein the antigen-binding molecule comprises a single domain antibody sequence incorporating the following CDRs:
CDR1 having the amino acid sequence of SEQ ID NO:2
CDR2 having the amino acid sequence of SEQ ID N0:10
CDR3 having the amino acid sequence of SEQ ID NO:11.

15. The antigen-binding molecule according to any one of claims 1 to 3 or claim 14, wherein the antigen-binding molecule comprises, or consists of, an amino acid sequence having at least 70% sequence identity to the amino acid sequence of SEQ ID NO:9.

16. The antigen-binding molecule according to any one of claims 1 to 13, wherein the antigen-binding molecule comprises a single domain antibody sequence incorporating the following FRs:
FR1 having the amino acid sequence of SEQ ID NO:40
FR2 having the amino acid sequence of SEQ ID NO:41
FR3 having the amino acid sequence of SEQ ID NO:42
FR4 having the amino acid sequence of SEQ ID NO:44.

17. The antigen-binding molecule according to any one of claims 1 to 3, claim 15 or claim 16, wherein the antigen-binding molecule comprises a single domain antibody sequence incorporating the following FRs:
FR1 having the amino acid sequence of SEQ ID NO:40
FR2 having the amino acid sequence of SEQ ID NO:41
FR3 having the amino acid sequence of SEQ ID NO:43
FR4 having the amino acid sequence of SEQ ID NO:44.

18. The antigen-binding molecule according to any one of claims 1 to 17, wherein the antigen-binding molecule inhibits interaction between VEGFA and VEGFR.

19. The antigen-binding molecule according to any one of claims 1 to 18, wherein the antigen-binding molecule is a multispecific antigen-binding molecule, further comprising an antigen-binding domain specific for a target antigen other than VEGFA.

20. A chimeric antigen receptor (CAR) comprising an antigen-binding molecule according to any one of claims 1 to 19.

21. A nucleic acid, optionally isolated, encoding an antigen-binding molecule according to any one of claims 1 to 19, or a CAR according to claim 20.

22. An expression vector comprising a nucleic acid according to claim 21.

23. A cell comprising an antigen-binding molecule according to any one of claims 1 to 19, a CAR according to claim 20, a nucleic acid according to claim 21, or an expression vector according to claim 22.

24. A method for producing an antigen-binding molecule which binds to VEGFA, comprising culturing a cell according to claim 23 under conditions suitable for expression of an antigen-binding molecule or CAR by the cell.

25. A composition comprising an antigen-binding molecule according to any one of claims 1 to 19, a CAR according to claim 20, a nucleic acid according to claim 21, an expression vector according to claim 22, or a cell according to claim 23, and a pharmaceutically acceptable carrier, diluent, excipient or adjuvant.

26. An antigen-binding molecule according to any one of claims 1 to 19, a CAR according to claim 20, a nucleic acid according to claim 21, an expression vector according to claim 22, a cell according to claim 23, or a composition according to claim 25, for use in a method of medical treatment or prophylaxis.

27. An antigen-binding molecule according to any one of claims 1 to 19, a CAR according to claim 20, a nucleic acid according to claim 21, an expression vector according to claim 22, a cell according to claim 23, or a composition according to claim 25, for use in a method of treatment or prevention of a disease in which VEGFA/VEGFR-mediated signalling is pathologically-implicated.

28. Use of an antigen-binding molecule according to any one of claims 1 to 19, a CAR according to claim 20, a nucleic acid according to claim 21, an expression vector according to claim 22, a cell according to claim 23, or a composition according to claim 25, in the manufacture of a medicament for treating or preventing a disease in which VEGFA/VEGFR-mediated signalling is pathologically-implicated.

29. A method of treating or preventing a disease in which VEGFA/VEGFR-mediated signalling is pathologically-implicated, comprising administering to a subject a therapeutically- or prophylactically-effective amount of an antigen-binding molecule according to any one of claims 1 to 19, a CAR according to claim 20, a nucleic acid according to claim 21, an expression vector according to claim 22, a cell according to claim 23, or a composition according to claim 25.

30. The antigen-binding molecule, CAR, nucleic acid, expression vector, cell or composition for use according to claim 27, the use according to claim 28, or the method according to claim 29, wherein the disease is selected from: a disease characterised by pathological angiogenesis, a cancer, a VEGFA-expressing cancer, a VEGFR-expressing cancer, an ocular disease, retinopathy, diabetic retinopathy, macular degeneration, age-related macular degeneration, wet age-related macular degeneration, retinal vein occlusion, myopic choroidal neovascularisation, retinopathy of prematurity, neovascular glaucoma, central serous retinopathy, ocular tumor, corneal neovascularisation, an inflammatory disease, an autoimmune disease, arthritis, rheumatoid arthritis, osteoarthritis, psoriasis, multiple sclerosis, sepsis, motor neuron disease and amyotrophic lateral sclerosis.

31. An in vitro complex, optionally isolated, comprising an antigen-binding molecule according to any one of claims 1 to 19 bound to VEGFA.

32. A method for detecting VEGFA in a sample, comprising contacting a sample containing, or suspected to contain, VEGFA with an antigen-binding molecule according to any one of claims 1 to 19, and detecting the formation of a complex of the antigen-binding molecule with VEGFA.

33. Use of an antigen-binding molecule according to any one of claims 1 to 19 in a method for detecting, localizing or imaging VEGFA, or cells comprising or expressing VEGFA.

34. A method of selecting or stratifying a subject for treatment with a VEGFA-targeted agent, the method comprising contacting, in vitro, a sample from the subject with an antigen-binding molecule according to any one of claims 1 to 19, and detecting the formation of a complex of the antigen-binding molecule with VEGFA.

35. Use of an antigen-binding molecule according to any one of claims 1 to 19 as an in vitro or in vivo diagnostic or prognostic agent.

36. Use of an antigen-binding molecule according to any one of claims 1 to 19 in a method for detecting, localizing or imaging a disease/condition characterised by expression of VEGFA.
